(19) United States
(12) Patent Application Publication
(10) Pub. No.: US 2016/0015786 A1
(43) Pub. Date: Jan. 21, 2016

(54) MOBILIZING AGENTS AND USES THEREFOR

(71) Applicant: MATER MEDICAL RESEARCH INSTITUTE LIMITED, South Brisbane, Qld, (AU)

(72) Inventors: Jean-Pierre Jose Rene Levesque, Yeerongpilly (AU); Ingrid Gabriele WINKLER, Yeerongpilly (AU)

(73) Assignee: MATER MEDICAL RESEARCH INSTITUTE LIMITED, South Brisbane, Qld, (AU)

(21) Appl. No.: 14/772,022

(22) PCT Filed: Nov. 4, 2013
(86) PCT No.: PCT/IB2013/003118

§ 371 Date: Sep. 1, 2015

Related U.S. Application Data
(60) Provisional application No. 61/771,373, filed on Mar. 1, 2013.

(51) Int. Cl. A61K 38/19; A61K 31/395; A61K 45/06

(52) U.S. Cl. CPC A61K 38/193; A61K 45/06; A61K 31/395

(57) ABSTRACT

Disclosed is the use of a HIF-α potentiating agent and a mobilizer of hematopoietic stem cells and/or progenitor cells in methods and compositions for mobilizing hematopoietic stem cell and progenitor cells from the bone marrow into the peripheral blood. The compositions and methods are particularly useful for stem cell transplantation and for treating or preventing immune deficiencies.

### CROSS REFERENCE TO RELATED APPLICATIONS

This application claims the benefit under 35 U.S.C. §119(e) of U.S. Provisional Application 61/771,373 filed Mar. 1, 2013 and is hereby incorporated by reference in its entirety.

### FIELD OF THE INVENTION

This invention relates generally to the use of a HIF-α potentiating agent and a mobilizer of hematopoietic stem cells and/or progenitor cells in methods and compositions for mobilizing hematopoietic stem cells and progenitor cells from the bone marrow into the peripheral blood. The present invention is particular relevant for stem cell transplantation as well as treating or preventing immune deficiencies.

Bibliographic details of certain publications numerically referred to in this specification are collected at the end of the description.

### BACKGROUND OF THE INVENTION

Autologous hematopoietic stem cell (HSC) transplantation is a curative treatment for various hematologic malignancies including leukemias, lymphomas and multiple myeloma. During the last decade, bone marrow aspiration has been progressively replaced by mobilized peripheral blood as a source of transplantable HSCs. The cytokine granulocyte colony-stimulating factor (G-CSF) is the main mobilizing agent used in the clinic. Administered subcutaneously daily at doses of 10 μg/kg, it promotes the forced egress of hematopoietic stem and progenitor cells (HSPCs) from the bone marrow into the circulation. In the vast majority of healthy allogeneic donors, CD34⁺ HSPCs are robustly mobilized after 4-5 days of G-CSF treatment and blood aphaeresis from day 5 is sufficient to reach the minimum threshold of 2×10⁶ CD34⁺ cells/kg body weight to ensure rapid reconstitution. However, in the autologous setting, up to 30-60% of chemotherapy-treated patients fail to reach this minimal threshold in response to G-CSF, precluding transplantation (1). Most at risk are patients who have undergone chemotherapy with purine analogs such as fludarabine for more than three chemotherapy cycles (2). The chemotactic interaction between the chemokine CXCL12 and its receptor CXCR4 are pivotal to HSPC retention within the bone marrow (3-4) and mobilization in response to G-CSF (5-6). Consequently, additional inhibition of the chemotactic interaction between CXCL12 and CXCR4 with specific small synthetic inhibitors such as Plerixafor (AMD3100) enhances synergistically HSPC mobilization in response to G-CSF in humans and mice (7). The synergistic effect of Plerixafor has been confirmed in at least two large phase 3 clinical trials with multiple myeloma and non-Hodgkin's lymphoma patients eligible for autologous HSC transplantation who previously failed to mobilize adequately in response to G-CSF alone. Plerixafor injected daily 1 hr prior to blood aphaeresis from day 4 of G-CSF administration enables approximately 60% of patients who previously failed to mobilize in response to G-CSF alone to reach the minimal threshold of 2×10^{δ} CD34⁺ cells/kg (8-9). However, the remaining 40% of patients who failed to mobilize in response to G-CSF alone, still fail to mobilize adequately with G-CSF and Plerixafor (8-9).

Accordingly, there is a pressing need for more effective approaches for mobilizing HSPCs from the bone marrow into the peripheral blood.

### SUMMARY OF THE INVENTION

The present invention is related in part to the discovery that mobilization of HSPCs by mobilizing agents (also referred to herein as “mobilizers” or “mobilizer of hematopoietic stem cells and/or progenitor cells”) such as G-CSF or Plerixafor, or combinations thereof, is significantly enhanced by co-administration of an agent that increases the activity of hypoxia-inducible factor α (HIF-α) (also referred to herein as a “HIF-α potentiating agent”). This in turn results in higher numbers of hematopoietic stem cells and/or progenitor cells including granulocytes/macrophage progenitors and/or megakaryocyte/erythrocyte progenitors in peripheral blood, when compared to administration of stem cell mobilizers alone. Concurrent administration of a HIF-α potentiating agent and a mobilizer of hematopoietic stem cells and/or progenitor cells is useful in compositions and methods for stimulating or enhancing mobilization of hematopoietic stem cells and/or progenitor cells or for stimulating or enhancing hematopoiesis, or for stem cell transplantation, as described hereafter.

Accordingly, in one aspect, the present invention provides compositions that comprise, consist or consist essentially of a HIF-α potentiating agent and at least one mobilizer of hematopoietic stem cells and/or progenitor cells. In another aspect, the invention provides a composition that comprises a HIF-α potentiating agent for use in mobilizing hematopoietic stem cells and/or progenitor cells. The HIF-α potentiating agent is selected, without limitation, from agents that stabilize HIP-α, agents that stimulate or enhance expression of HIF-α HIF-α polypeptide or coding sequences, and combinations thereof. In some embodiments, the HIF-α potentiating agent inhibits the activity of a HIF hydroxylase, e.g., a HIF prolyl hydroxylase (PHD) (e.g., PHD1, PHD2 and/or PHD3). Such agents are also referred to herein as “PHD inhibitors” or “PHI.” In specific embodiments, the PHD inhibitor is a selective inhibitor of a HIF-α PHD. In some embodiments, the PHD inhibitor is an inhibitor of two or more PHD enzymes. In some embodiments, the at least one mobilizer is selected from a growth factor, a cytokine, a chemokine or a polysaccharide. Suitably, the at least one mobilizer is characterized by its ability to decrease or block the expression, synthesis or function of CXCL12 or is characterized by its ability to block or antagonize CXCR4. In specific embodiments, the mobilizer is selected from a colony stimulating factor such as G-CSF or a variant, derivative or analog thereof, a CXCR4 antagonist such as Plerixafor, or a combination thereof. In some embodiments, the compositions further comprise a pharmaceutically acceptable carrier.

The compositions of the present invention are useful for stimulating or enhancing mobilization of hematopoietic stem cells and/or progenitor cells, or for stimulating or enhancing hematopoiesis, or for stem cell transplantation. Accordingly, in a related aspect, the present invention provides a use of a HIF-α potentiating agent for stimulating or enhancing mobilization of hematopoietic stem cells and/or progenitor cells, or for stimulating or enhancing hematopoiesis, or for stem cell transplantation, or for treating or preventing an immunocompromised condition (e.g., neutropenia, agranulocytosis, thrombocytopenia, or anemia). In this aspect, the use is in subjects that have been, are, or will be administered at least one mobilizer of hematopoietic stem cells and/or progenitor cells. In some embodiments, the HIF-α potentiating agent and the at least one mobilizer are prepared or manufactured as medicaments for those applications.

Another aspect of the present invention provides methods for enhancing a hematopoietic function of a mobilizer of hematopoietic stem cells and/or progenitor cells in a subject. These methods generally comprise, consist or consist essentially of administering to the subject a HIF-α potentiating agent in an effective amount to enhance an hematopoietic function of the mobilizer (e.g., increasing the number of hematopoietic stem cells and/or progenitor cells including granulocytes/macrophage progenitors and/or megakaryocyte/erythrocyte progenitors in the peripheral blood).

Yet another aspect of the present invention provides methods for mobilizing hematopoietic stem cells and/or progenitor cells from bone marrow into peripheral blood of a donor subject. These methods generally comprise, consist or consist essentially of: administering to the subject a HIF-α potentiating agent in an effective amount to mobilize hematopoietic stem cells and/or progenitor cells from the bone marrow into the peripheral blood of the subject. The donor subjects in these embodiments are selected from subjects that have been, are, or will be administered at least one mobilizer of hematopoietic stem cells and/or progenitor cells. A related method generally comprises, consists or consists essentially of: administering concurrently to the donor subject a HIF-α potentiating agent and at least one mobilizer of hematopoietic stem cells and/or progenitor cells in effective amounts to mobilize hematopoietic stem cells and/or progenitor cells from the bone marrow into the peripheral blood of the subject. Suitably, the HIF-α potentiating agent is administered in an amount that is effective to enhance the hematopoietic function of the at least one mobilizer. In specific embodiments, the HIF-α potentiating agent and the at least one mobilizer are administered in synergistically effective amounts. In some embodiments, the subject has an immunocompromised condition or is at risk or acquiring an immunocompromised condition (e.g., the subject will be exposed to an agent or treatment that gives rise or is likely to give rise to an immunocompromised condition). In illustrative examples of this type, the subject has a hyperproliferative cell disorder (e.g., cancer, which can be a primary cancer or a metastatic cancer, or an autoimmune disease), and has been, is or will be subjected to a medical treatment. In specific embodiments, the hyperproliferative cell disorder is cancer (e.g., leukemia, multiple myeloma, lymphoma, etc.). Suitably, the medical treatment targets rapidly dividing cells or disrupts the cell cycle or cell division (e.g., chemotherapy and/or radiation therapy). Suitably, the immunocompromised condition is selected from neutropenia, agranulocytosis, thrombocytopenia, and anemia.

In some embodiments, the methods further comprise collecting or harvesting mobilized hematopoietic stem cells and/or progenitor cells from the donor subject (e.g., from the subject's peripheral circulation). In some of these embodiments, the methods further comprise culturing and/or storing the collected or harvested mobilized hematopoietic stem cells and/or progenitor cells (e.g., to maintain or expand the collected or harvested mobilized hematopoietic stem cells and/or progenitor cells). In some embodiments, the methods further comprise infusing or transplanting the collected or harvested mobilized hematopoietic stem cells and/or progenitor cells, which have been optionally cultured or stored, into a recipient subject. The donor subject and the recipient subject may be the same subject or may be different subjects. In some embodiments, the subject is both a donor and a recipient of the collected or harvested mobilized hematopoietic stem cells and/or progenitor cells, which have been optionally cultured or stored, and is suitably in need of a stem cell transplantation. The stem cell transplantation in these embodiments is autologous with respect to the recipient. Suitably, the subject has an immunocompromised condition or has been exposed to a medical treatment that results in an immunocompromised condition.

In other embodiments, the methods further comprise infusing or transplanting the collected or harvested mobilized hematopoietic stem cells and/or progenitor cells, which have been optionally cultured or stored, into another subject. In these embodiments the donor subject and the recipient subject are different subjects. In these embodiments, the subject from which the mobilized hematopoietic stem cells and/or progenitor cells are collected or harvested is a donor and the other subject is a recipient that is suitably in need of a stem cell transplantation. The stem cell transplantation in these embodiments is allogeneic or xenogeneic with respect to the recipient. Suitably, the other (recipient) subject has an immunocompromised condition or has been exposed to a medical treatment that results in an immunocompromised condition.

In some embodiments, the methods further comprise administering to the recipient prior to, simultaneously with, or after the stem cell transplantation a HIF-α potentiating agent and a mobilizer of hematopoietic stem cells and/or progenitor cells in effective amounts to mobilize hematopoietic stem cells and/or progenitor cells from the bone marrow into the peripheral blood of the subject.

In accordance with the present invention, HIF-α potentiating agents are useful for enhancing a hematopoietic function (e.g., increasing the number of hematopoietic stem cells and/or progenitor cells, including granulocytes/macrophage progenitors and/or megakaryocyte/erythrocyte progenitors in the peripheral blood) of at least one mobilizer of hematopoietic stem cells and/or progenitor cells. The HIF-α potentiating agent may be known, or identified using any suitable screening assay. Accordingly, in a related aspect, the present invention provides screening methods for identifying agents that are useful for enhancing a henmatopoietic function of the mobilizer. These methods generally comprise testing whether a test agent potentiates HIF-α (e.g., increasing the accumulation of, or stability of, HIF-α; directly provide HIF-α activity; or increase expression of HIF-1) and determining whether the test agent stimulates or enhances mobilization of hematopoietic stem cells and/or progenitor cells on the basis that it tests positive for the potentiation.

In another related aspect, the present invention provides methods of producing an agent that enhances a hematopoietic function of at least one mobilizer of hematopoietic stem cells and/or progenitor cells. These methods generally comprise: identifying a HIF-α potentiating agent by a screening assay as broadly described above; and synthesizing the agent on the basis that it tests positive for enhancing a hematopoietic function of the mobilizer. Suitably, the method further comprises derivatizing the agent, and optionally formulating the derivatized agent with a pharmaceutically acceptable carrier, to improve the efficacy of the agent for enhancing the hematopoietic function of the mobilizer.

The mobilizer(s) and the HIF-α potentiating agent are suitably administered in the form of one or more compositions each comprising a pharmaceutically acceptable carrier. The composition(s) may be administered by injection, by topical application or by the oral route including sustained-release modes of administration, over a period of time and in amounts which are effective for increasing the number of hematopoietic stem cells and/or progenitor cells including granulocytes/macrophage progenitors and/or megakaryocyte/erythrocyte progenitors in the peripheral blood.

In some embodiments, the mobilizer(s) and the HIF-α potentiating agent are administered simultaneously to the subject. In other embodiments the HIF-α potentiating agent is administered to the subject prior to administration of the mobilizer. In still other embodiments, the HIF-α potentiating agent is administered after administration of the mobilizer to the subject.

In a related aspect, the methods are useful for treating or preventing an immunocompromised condition in a subject (e.g., a condition resulting from exposure of the subject to a medical treatment). In these embodiments, the mobilizer(s) and the HIF-α potentiating agent are concurrently administered in amounts effective for treatment or prevention of the immunocompromised condition (e.g., neutropenia, agranulocytosis, thrombocytopenia, or anemia). In some of these embodiments, the methods further comprise identifying a subject having or at risk of acquiring the immunocompromised condition. Suitably, the HIF-α potentiating agent and the mobilizer(s) may be administered to the subject simultaneously, sequentially or separately with the medical treatment. In some embodiments, the concurrent administration of the HIF-α potentiating agent and the mobilizer(s) is a prophylactic treatment (e.g., the subject is preparing to undergo chemotherapy or radiation treatment). In others, it is a therapeutic treatment (e.g., the subject has received at least one dose of chemotherapy or at least one radiation treatment).

In some embodiments, the methods may further comprise exposing the subject to an ancillary treatment that treats or prevents an immunocompromised condition. In illustrative examples of this type, the immunocompromised condition is anemia and the ancillary treatment may comprise administering to the subject an anemia medicament selected from recombinant erythropoietin (EPO), ferrous iron, ferric iron, vitamin B12, vitamin B6, vitamin C, vitamin D, calcitriol, alphacalcidol, folate, androgen, and carnitine. In other illustrative examples, the immunocompromised condition is thrombocytopenia and the ancillary treatment may comprise administering to the subject a thrombocytopenia medicament selected from a glucocorticoid, recombinant thrombopoictin (TPO), recombinant megakaryocyte growth and development factor (MGDF), PEGylated recombinant MGDF and lisophylline. In still other illustrative examples, the immunocompromised condition is neutropenia and the ancillary treatment suitably comprises administering to the subject a neutropenia medicament selected from glucocorticoid, immunoglobulin, androgens, recombinant IFN-γ, and uteroferrin. In some embodiments, the ancillary treatment is administered to the subject simultaneously, sequentially or separately with the HIF-α potentiating agent and/or the mobilizer(s).

In some embodiments, the medical treatment is likely to expose the subject to a higher risk of infection. Accordingly, in these embodiments, the methods may further comprise administering simultaneously, sequentially or separately with the HIF-α potentiating agent and/or the mobilizer(s) at least one anti-infective agent that is effective against an infection that develops or that has an increased risk of developing from the immunocompromised condition, wherein the anti-infective agent is selected from antimicrobials, antibiotics, antivirals, antifungals, anthelmintics, antiprotozoals and nematocides.

Typically, one or both of the HIF-α potentiating agent and the at least one mobilizer are administered on a routine schedule, for example, every day, at least twice a week, at least three times a week, at least four times a week, at least five times a week, at least six times a week, every week, every other week, every third week, every fourth week, every month, every two months, every three months, every four months, and every six months.

In some advantageous embodiments, the concurrent administration of a HIF-α potentiating agent and at least one mobilizer of hematopoietic stem cells and/or progenitor cells) is useful for treating or preventing hematopoietic disorders such as neutropenia, agranulocytosis, thrombocytopenia, and anemia, which may result, for example, from myclosuppressive, myeloablative or cytoreductive treatments that target rapidly dividing cells or that disrupt the cell cycle or cell division (e.g., chemotherapy or radiation therapy). Accordingly, in yet another aspect, the present invention provides methods for treating a hyperproliferative cell disorder (e.g., a cancer or autoimmune disorder) in a subject. These methods generally comprise administering concurrently to the subject a medical treatment (e.g., a chemotherapeutic agent or radiation) for the disorder, which targets rapidly dividing cells or disrupts the cell cycle or cell division, together with at least one mobilizer of hematopoietic stem cells and/or progenitor cells and a HIF-α potentiating agent in amounts effective for stimulating or enhancing mobilization of hematopoietic stem cells and/or progenitor cells, or for stimulating or enhancing hematopoiesis.

Since administration of the combination treatment of the present invention will reduce the risk of having or developing a hematopoietic disorder as a side effect of the myeloablative, myelosuppressive or cytoreductive treatment, it is possible to administer higher therapeutic doses of a chemotherapeutic agent or radiation to a subject in order to kill or inhibit the growth or proliferation of a tumor or to treat or prevent an autoimmune disease in the subject. Accordingly, in yet another aspect, the present invention provides methods for increasing the dose in a subject of a medicament for treating a hyperproliferative cell disorder (e.g., cancer or an autoimmune disease), wherein the medicament results or increases the risk of developing an immunocompromised condition. These methods generally comprise administering concurrently the medicament to the subject in a dose that ordinarily induces side effects (e.g., the development of the immunocompromised condition), together with at least one mobilizer of hematopoietic stem cells and/or progenitor cells and a HIF-α potentiating agent in amounts effective for inhibiting or preventing the induction of those side effects (e.g., in amounts effective for increasing the number of hematopoietic stem cells and/or progenitor cells including granulocytes/macrophage progenitors and/or megakaryocyte/erythrocyte progenitors in the peripheral blood).

In yet another aspect, the present invention provides pharmaceutical compositions for treating or preventing a hyperproliferative cell disorder (e.g., cancer or an autoimmune disease) that is treatable or preventable by a medical treatment that targets rapidly dividing cells or that disrupts the cell cycle or cell division (e.g., chemotherapy or radiation therapy). These compositions generally comprise, consist or consist essentially of a HIF-α potentiating agent, at least one mobilizer of hematopoietic stem cells and/or progenitor cells and at least one other agent selected from a chemotherapeutic agent (e.g., a cytotoxic agent), a radiosensitizing agent, an anemia medicament, a thrombocytopenia medicament, a neutropenia medicament, an agranulocytosis medicament and an anti-infective agent, and optionally a pharmaceutically acceptable carrier.

### BRIEF DESCRIPTION OF THE DRAWINGS

FIGS. 1A, 1B, 1C, and 1D are graphical representations showing the effect of Compound X and G-CSF on HIF-1α protein and CFC mobilization. (1A) Western blot analysis of bone marrow (BM) cell lysates from mice treated with saline (Saline), with Compound X for 3 days (X3), with G-CSF for 2 days (G2) or with both Compound X and G-CSF (G2X3) for presence of HIF-1α and β-actin. Each lane represents a different mouse. (1B) Graph shows the numbers of CFC per mL blood and per spleen for mice treated with G-CSF (open diamonds) or with Compound X for 3 days and G-CSF (filled squares) for indicated numbers of days. (1C) Graph shows the numbers of CFC per mL blood and per spleen for mice treated with G-CSF for 2 days and with Compound X for 0 to 4 days. (1D) Time-course numbers of CFC per mL blood or per spleen for mice treated with Plerixafor with or without Compound X. Mice were treated with Plerixafor for 1 hour (P1) together with Compound X for 1, 2, 3 or 4 days (P1X1-P1X4). Data are mean±SD of 6 mice per condition. * p<0.05; ** p<0.01; *** p<0.001.

FIGS. 2A, 2B, 2C, and 2D present graphical representations showing a synergistic effect of Compound X with G-CSF, or in combination with G-CSF and Plerixafor. (2A) Treatment groups used. (2B) Graphs show the number of CFC per ml blood and per spleen of treated mice, GCSF only (filled circles); GCSF and Compound X (open squares); GCSF and Plerixafor (filled triangles); and the combination of GCSF, Plerixafor and Compound X (filled diamonds). (2C) Timecourse of the number of Lin⁻Sca1⁺ Kit⁺ HSPCs and (2D) Lin⁻Sca1⁺ Kit⁺ CD48-CDL50⁺ HSCs per mL of blood or per spleen following treatments with combinations of G-CSF, Plerixafor and Compound X as in 2A, GCSF only (filled circles); GCSF and Compound X (open squares); GCSF and Plerixafor (filled triangles); and the combination of GCSF, Plerixafor and Compound X (filled diamonds). Data are mean±SD of 6 mice per time-point per treatment group. *: p<0.05; ** 0.001<p<0.01; *** p<0.001.

FIGS. 3A and 3B are graphical representations showing that Compound X synergizes with G-CSF and Plerixafor to enhance mobilization of competitive repopulating HSCs. CD45.2⁺ mice were mobilized with G-CSF for 2 or 4 days with G-CSF alone (filled circles), with G-CSF in combination with Plerixafor for 1 hr (filled triangles), with Compound X for 3 days (filled squares) or with both Plerixafor, 1 hr, and Compound X, 3 days (filled inverted triangles). Blood (20 μL) was transplanted in lethally irradiated recipients together with 200,000 competitive BM cells from CD45.1⁺ mice. Proportion of blood CD45.2+ leukocytes at 16 weeks post-transplantation and number of repopulating units per mL of mobilized blood calculated from donor chimerism at 16 weeks post-transplantation from (3A) 2 days treatment with G-CSF and (3B) 4 days treatment with G-CSF. Each dot is a result from one recipient mouse, bars are average. *: p<0.05; ** 0.001<p<0.01; *** p<0.001.

FIGS. 4A, 4B, 4C, and 4D are graphical representations showing that deletion of Hif1a gene in HSPCs compromises HSPC mobilization in response to G-CSF. CreER was activated in mutant mice in which both Hif1a alleles are floxed to delete the Hif1a genes and the mice were mobilized for 3 days with G-CSF. (4A) Proportion of Lin⁻Sca1⁺Kit⁺ CD48⁻CD150⁺ HSCs, Lin⁻Sca1⁺Kit⁺ HSPCs, Lin⁻Sca1⁻Kit⁺ myeloid progenitors and Lin⁺ leukocytes in which yellow fluorescent protein (YFP) is induced by Cre ER following a 3 day tamoxifen treatment in Hif1a^{fl/fl} R26R^{YFP/YFP} SclCreER mice. Number of (4B) CFCs and (4C) Lin⁻Sca1⁺Kit⁺CD48⁻ CD150⁺ HSCs, Lin⁻Sca1⁺Kit⁺ HSPCs mobilized in blood and spleen in Hif1a^{fl/fl} R26R^{YFP/YFP} SclCreER mice (solid circles or squares) and control Hif1a^{WT/WT} R26R^{YFP/YFP} SclCreER mice (open circles or squares). In 4B each dot represents a separate mouse. p<0.05; ** 0.001<p<0.01; *** p<0.001. (4D) Proportion of YFP (Hif1a^{fl/fl} non-deleted) and YFP⁺ (Hif1a^{Δ/Δ} deleted) HSCs mobilized to the blood or spleen from the bone marrow versus YFP− or YFP+ HSCs remaining in the bone marrow following a CreER induction with tamoxifen and a 3 day G-CSF treatment. Pairs of dots show results for YFP− and YFP+ HSC within the same mobilized Hif1a^{fl/fl} R26R^{YFP/YFP} SclCreER mouse. Significance were calculated with a paired t-test.

FIGS. 5A, 5B, and 5C are graphical representations showing that deletion of Hif1a gene in osteoprogenitors delays HSPC mobilization in response to G-CSF. (5A) Proportion of CD45−Lin−CD31−Sca1−51+ osteoblasts and CD45−Lin−CD31+ endothelial cells in which YFP is induced by Cre ER in Hif1a^{fl/fl} R26R^{YFP/YFP} OsxCreER mice. (5B) Number of CFCs and Lin⁻Sca1⁺Kit⁺ CD48⁻ HSCs, Lin⁻Sca1⁺Kit⁺ HSPCs per femur in Hif1a^{fl/fl} R26R^{YFP/YFP} OsxCreER mice, (HIF 1a wt, open circles, HIF 1a fl/fl, solid circles). (5C) Number of CFCs mobilized in blood and spleen in Hif1a^{fl/fl} R26R^{YFP/YFP} OsxCreER mice following 2-4 days treatment with G-CSF (HIF 1a wt, open circles, HIF 1a fl/fl, solid circles).

FIGS. 6A, and 6B are graphical representations demonstrating the effect of treatment with Compound A, Compound B, or Compound C in combination with G-CSF on the number and phenotypic distribution of hematopoietic stem cells and progenitor cells in the bone marrow. Treatment with G-CSF and vehicle control served as control. (6A) Myeloid progenitors (LKS−)—Top Panel, hematopoietic stem and progenitor cells (LKS+)—Middle Panel, and LKS+ CD48+ lineage-restricted progenitors—Bottom Panel. (6B) LKS+ CD48−CD150− multipotent progenitors—Top Panel, and LSK+ CD48− CD150+ hematopoietic stem cells—Bottom Panel.

FIGS. 7A and 7B are graphical representations demonstrating the effect of treatment with Compound A, Compound B or Compound C in combination with G-CSF on mobilization to the blood of hematopoietic stem cells and progenitor cells. Treatment with G-CSF and vehicle control served as control. (7A) phenotypic myeloid progenitors (LKS−)—Top Panel, hematopoietic stem and progenitor cells (LKS+)—Middle Panel, and LKS+CD48+ lineage-restricted progenitors—Bottom Panel. (TB) LKS+CD48−CD150− multipotent progenitors—Top Panel, and LKS+CD48− CD150+ hematopoietic stem cells—Bottom Panel. * p<0.05; ** p<0.01; ***p<0.001.

FIGS. 8A and 8B are graphical representations demonstrating the effect of treatment with Compound A, Compound B or Compound C in combination with G-CSF on mobilization to the spleen of hematopoietic stem cells and progenitor cells. Treatment with G-CSF and vehicle control served as control. (8A) phenotypic myeloid progenitors (LKS−)—Top Panel, hematopoietic stem and progenitor cells (LKS+)—Middle Panel, and LKS+CD48+ lineage-restricted progenitors—Bottom Panel. (8B) LKS+ CD48−CD150− multipotent progenitors—Top Panel, and LKS+CD48− CD150+hematopoietic stem cells—Bottom Panel. * p<0.05; ** p<0.01; ***p<0.001.

FIGS. 9A and 9B are graphical representations demonstrating the effect of treatment with Compound A, Compound B or Compound C in combination with G-CSF on total mobilization per mouse (blood and spleen) of hematopoietic stem cells and progenitor cells. Treatment with G-CSF and vehicle control served as control. (9A) phenotypic myeloid progenitors (LKS−)—Top Panel, hematopoletic stem and progenitor cells (LKS+)—Middle Panel, and LKS+CD48+ lineage-restricted progenitors—Bottom Panel. (9B) LKS+CD48− CD150− multipotent progenitors—Top Panel, and LKS+CD48− CD150+ hematopoietic stem cells—Bottom Panel. * p<0.05; ** p<0.01; ***p<0.001.

FIG. 10 is a graphical representation demonstrating the effect of treatment with Compound A, Compound B or Compound C in combination with G-CSF on colony forming unit (CFU) mobilization to the blood—Top Panel, spleen—Middle Panel, and combined total mobilized (blood and spleen)—Bottom Panel. Treatment with G-CSF and vehicle control served as control. * p<0.05; ** p<0.01; ***p<0.001.

FIG. 11 is a graphical representation demonstrating the effect of treatment with Compound A, Compound B or Compound C in combination with G-CSF on white blood cell (WBC) number per ml of blood—Top Panel, and spleen weight—Bottom Panel Treatment with G-CSF and vehicle control served as control. * p<0.05; ** p<0.01; ***p<0.001.

FIGS. 12A and 12 B are graphical representations demonstrating the effect of treatment with Compound D, Compound E or Compound F in combination with G-CSF on total mobilization to blood and spleen of hematopoietic stem cells and progenitor cells. Treatment with G-CSF and vehicle control served as control. (12A) phenotypic myeloid progenitors (LKS−)—Top Panel, hematopoietic stem and progenitor cells (LKS+)—Middle Panel, and LKS+CD48+ lineage-restricted progenitors—Bottom Panel. (12B) LKS+CD48− CD150− multipotent progenitors—Top Panel, and LKS+ CD48− CD150+ hematopoietic stem cells—Bottom Panel. * p<0.05; ** p<0.01; ***p<0.001.

FIG. 13 is a graphical representation demonstrating the effect of Compound D, Compound E or Compound F in combination with G-CSF on colony forming unit (CFU) mobilization to the blood—Top Panel, spleen—Middle Panel, and combined total (blood and spleen)—Bottom Panel. Treatment with G-CSF and vehicle control served as control. * p<0.05; ** p<0.01; ***p<0.001.

FIGS. 14A and 14B are graphical representations demonstrating the effect of treatment with Compound H, Compound J or Compound K in combination with G-CSF on the total mobilization to blood and spleen of hematopoietic stem cells and progenitor cells. Treatment with G-CSF and vehicle control served as control. (14A) phenotypic myeloid progenitors (LKS−)—Top Panel, hematopoietic stem and progenitor cells (LKS+)—Middle Panel, and LKS+CD48+ lineage-restricted progenitors—Bottom Panel. (14B) LKS+ CD48−CD150− multipotent progenitors—Top Panel, and LKS+CD48− CD150+ hematopoietic stem cells—Bottom Panel. * p<0.05; ** p<0.01; ***p<0.001.

FIG. 15 is a graphical representation demonstrating the effect of treatment with Compound H, Compound J or Compound K in combination with G-CSF on colony forming unit (CFU) mobilization to the blood—Top Panel, spleen—Middle Panel, and combined total (blood and spleen)—Bottom Panel. * p<0.05; ** p<0.01; ***p<0.001

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “a mobilizer of hematopoietic stem cells and/or progenitor cells” means one mobilizer of hematopoietic stem cells and/or progenitor cells or more than one mobilizer of hematopoietic stem cells and/or progenitor cells.

By “about” is meant a quantity, level, value, number, frequency, percentage, dimension, size, amount, weight or length that varies by as much as 10% to a reference quantity, level, value, number, frequency, percentage, dimension, size, amount, weight or length to which it refers.

The terms “administered concurrently” or “administering concurrently” or “co-administering” and the like refer to the administration of a single composition containing two or more active agents, or the administration of each active agent as separate compositions and/or delivered by separate routes either contemporaneously or simultaneously or sequentially within a short enough period of time that the effective result is equivalent to that obtained when all such active agents are administered as a single composition, or that the result achieved is the combined effect of agents. For example, a HIF-α potentiating agent may be administered together with a mobilizer of hematopoietic stem cells and/or progenitor cells in order to increase the numbers of hematopoietic stem cells, progenitor cells and/or differentiated cells thereof in peripheral blood. In another example, a HIF-α potentiating agent and a mobilizer of hematopoietic stem cells and/or progenitor cells are administered together with another agent to enhance their effects or to ameliorate the effects of a medical treatment that gives rise or contributes to an immunocompromised condition. In another example, a HIF-α potentiating agent is administered at a later point in time than a mobilizer of hematopoietic stem cells and/or progenitor cells but within the time period during which the mobilizer of hematopoietic stem cells and/or progenitor cells is still exerting an effect. By “sequential” administration is meant a time difference of from seconds, minutes, hours or days between the administration of the two types of molecules or active agents. These molecules or active agents may be administered in any order. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and preferably within less than about one to about four hours. In certain embodiments, the HIF-α potentiating agent and the mobilizer are administered within about 60 minutes, about 50 minutes, about 40 minutes, about 30 minutes, about 20 minutes, about 10 minutes, about S minutes, or about 1 minute of each other or separated in time by about 1 hour, about 2 hours, about 4 hours, about 6 hours, about 10 hours, about 12 hours, about 24 hours, about 36 hours, or about 72 hours, or more. When administered contemporaneously, the agents may suitably be administered at the same site on the subject. The term “same site” includes the exact location, but can be within about 0.5 to about 15 centimeters, usually from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound, composition, or molecule that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell which is capable of producing and secreting polypeptides referred to herein as well as a polynucleotide comprising a nucleotide sequence that encodes this polypeptide. Thus, the term “agent” extends to nucleic acid constructs including vectors such as viral or non-viral vectors, expression vectors and plasmids for expression in and secretion in a range of cells.

The term “acyl” denotes a group containing the moiety C═O (and not being a carboxylic acid, ester or amide or thioester). Preferred acyl includes C(O)—R, wherein R is hydrogen or an alkyl, alkenyl, alkynyl, aryl, cycloalkyl, heterocyclyl, arylalkyl, cycloalkylalkyl or heterocyclylalkyl residue, suitably a C₁₋₂₀ residue. Non-limiting examples of acyl include formyl; straight chain or branched alkanoyl such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; phenylcarbonyl; cycloalkylcarbonyl such as cyclopropylmethyl(or ethyl)carbonyl cyclobutylmethyl(or ethyl)carbonyl, cyclopentylmethyl(or ethyl)carbonyl and cyclohexylmethyl (or ethyl)carbonyl; alkanoyl such as phenylalkanoyl (e.g., phenylacetyl, i.e., benzoyl, phenylpropanoyl, phenylbutanoyl, phenylpentanoyl, phenylhexanoyl) and naphthylalkanoyl (e.g., naphthylacetyl, naphthylpropanoyl and naphthylbutanoyl), and phenylalkenoyl (e.g., phenylhex-4-en-oyl, phenylhex-3-en-oyl, phenylheptanoyl, phenylhept-4-en-oyl, phenylhept-3-en-oyl).

An “agranulocytosis medicament” as used herein refers to a composition of matter which reduces the symptoms related to agranulocytosis, prevents the development of agranulocytosis, or treats existing agranulocytosis.

The term “alkenyl” as used herein denotes groups formed from straight chain or branched hydrocarbon residues containing at least one carbon to carbon double bond including ethylenically mono-, di- or poly-unsaturated alkyl groups as defined herein, suitably C₁₋₂₀ alkenyl (e.g., C₁₋₁₀ or C₁₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 4-heptenyl, 5-heptenyl, 3-hexenyl, 4-hexenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, and 1,4-hexadienyl. An alkenyl group may be optionally substituted by one or more optional substituents as herein defined. Accordingly, “alkenyl” as used herein is taken to refer to optionally substituted alkenyl.

As used herein, the term “alkyl,” when used alone or in words such as “arylalkyl,” “heterocyclylalkyl” and “cycloalkylalkyl,” denotes straight chain or branched hydrocarbon residues, suitably C₁₋₂₀ alkyl, e.g., C₁₋₁₀ or C₁₋₆. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methoxyhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propylocytl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. An alkyl group may be optionally substituted by one or more optional substituents as herein defined. Accordingly, “alkyl” as used herein is taken to refer to optionally substituted alkyl.

The term “alkynyl” denotes groups formed from straight chain or branched hydrocarbon residues containing at least one carbon to carbon triple bond including ethynyically mono-, di- or poly-unsaturated alkyl or cycloalkyl groups as defined herein, suitably C₁₋₂₀ alkynyl (e.g., C₁₋₁₀ or C₁₋₆). Examples, include ethynyl, propynyl, butynyl, pentynyl. An alkynyl group may be optionally substituted by one or more optional substituents as herein defined. Accordingly, “alkynyl” as used herein is taken to refer to optionally substituted alkynyl.

The term “aryl” used either alone or in compounds words such as “arylalkyl” and “aryloxy,” denotes single, polynuclear, conjugated or fused residues of aromatic hydrocarbons. Examples of aryl include phenyl, biphenyl and naphthyl. In specific embodiments, aryl groups include phenyl and naphthyl. An aryl group may be optionally substituted by one or more optional substituents as herein defined. Accordingly, “aryl” as used herein is taken to refer to aryl that may be optionally substituted, such as optionally substituted phenyl and optionally substituted naphthyl.

The terms “arylalkyl,” “cycloalkylalkyl” and “heterocyclylalkyl” refer to an alkyl group substituted (suitably terminally) by an aryl, cycloalkyl or heterocyclyl group, respectively.

The terms “aryloxy,” “cycloalkyloxy” and “heterocyclyloxy” denote aryl, cycloalkyl and heterocyclyl groups, respectively, when linked by an oxygen atom.

An “anemia medicament” as used herein refers to a composition of matter which reduces the symptoms related to anemia, prevents the development of anemia, or treats existing anemia.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in an animal, suitably a mammal, to which it is administered, wherein the response includes the production of elements which specifically bind the polypeptide or fragment thereof.

Reference herein to “bacteria” or “bacterial infection” includes any bacterial pathogen including emerging bacterial pathogen of vertebrates. Representative bacterial pathogens include without limitation species of: Acinetobacter, Actinobacillus, Actinomycetes, Actinomyces, Aeromonas, Bacillus, Bacteroides, Bordetella, Borrelia, Brucella (brucellosis), Burkholderia, Campylobacter, Citrobacter, Clostridium, Corynebacterium, Enterobacter, Enterococcus, Erysipelothrix, Escherichia, Francisella, Haemophilus, Helicobacter, Klebsiella, Legionella, Leptospira, Listeria, Micrococcus, Moraxella, Morganella, Mycobacterium (tuberculosis), Nocardia, Neisseria, Pasteurella, Plesiomonas, Propionibacterium, Proteus, Providencia, Pseudomonas, Rhodococcus, Salmonella, Serratia, Shigella, Staphylococcus, Stenotrophomonas, Streptococcus, Treponema, Vibrio (cholera) and Yersinia (plague).

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The term “colony stimulating factor” refers to a secreted glycoprotein that binds to receptor proteins on the surface of hematopoietic cells activating intracellular signalling pathways that cause the cells to proliferate and differentiate into different types of blood cells. CSF-1 (macrophage colony stimulating factor), CSF-2 (granulocyte macrophage colony stimulating factors; GM-CSF; sargramostim), and CSF-3 (granulocyte colony stimulating factors; G-CSF; filgrastim), and promegapoietin are examples of colony stimulating factors.

By “corresponds to” or “corresponding to” is meant a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

“Cycloalkyl” when used alone or in compound words such as “cycloalkoxy,” refers to cyclic hydrocarbon residues, including mono- or polycyclic alkyl groups. Exemplary cycloalkyl are C₄₋₇ alkyl. A “cycloalkyl” group may contain one or more double or triple bonds to form a cycloalkenyl or cycloalkynyl group and accordingly, “cycloalkyl” also refers to non-aromatic unsaturated as well as saturated cyclic hydrocarbon residues. Examples of “cycloalkyl” include cyclopropyl, cyclobutyl, cyclopentyl, cyclohexyl, cycloheptyl, cyclooctyl, cyclononyl, cyclodecyl, 1,3-cyclohexadienyl, 1,4-cyclohexadienyl, 1,3-cycloheptadienyl, 1,3,5-cycloheptatrienyl and 1,3,5,7-cyclooctatetraenyl. A cycloalkyl group may be optionally substituted by one or more optional substituents as herein defined. Accordingly, “cycloalkyl” as used herein is taken to refer to optionally substituted cycloalkyl.

The term “derivatize,” “derivatizing” and the like refer to producing or obtaining a compound from another substance by chemical reaction, e.g., by adding one or more reactive groups to the compound by reacting the compound with a functional group-adding reagent, etc.

The term “derivative,” in the context of polypeptide derivatives, refers to a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

The term “differentiation” of hematopoietic stem cells and/or hematopoietic progenitors as used herein refers to both the change of hematopoietic stem cells into hematopoietic progenitors and the change of hematopoietic progenitors into unipotent hematopoietic progenitors and/or cells having characteristic functions, namely mature cells including erythrocytes, leukocytes (e.g., neutrophils) and megakaryocytes. Differentiation of hematopoietic stem cells into a variety of blood cell types involves sequential activation or silencing of several sets of genes. Hematopoietic stem cells typically choose either a lymphoid or myeloid lineage pathway at an early stage of differentiation.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “expression” with respect to a gene sequence refers to transcription of the gene and, as appropriate, translation of the resulting mRNA transcript to a protein. Thus, as will be clear from the context, expression of a coding sequence results from transcription and translation of the coding sequence. Conversely, expression of a non-coding sequence results from the transcription of the non-coding sequence.

As used herein, the term “function” refers to a biological, enzymatic, or therapeutic function.

The term “gene” as used herein refers to any and all discrete coding regions of the cell's genome, as well as associated non-coding and regulatory regions. The term is intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

The term “group” as applied to chemical species refers to a set of atoms that forms a portion of a molecule. In some instances, a group can include two or more atoms that are bonded to one another to form a portion of a molecule. A group can be monovalent or polyvalent (e.g., bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e., H) added, and a negatively charged group can be envisioned as a neutral group with one or more protons removed. Non-limiting examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups. Groups such as alkyl, alkenyl, alkynyl, aryl, and heterocyclyl, whether used alone or in a compound word or in the definition of a group may be optionally substituted by one or more substituents. “Optionally substituted,” as used herein, refers to a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, phenylamino, diphenylamino, benzylamino, dibenzylamino, hydrazino, acyl, acylamino, diacylamino, acyloxy, heterocyclyl, heterocycloxy, heterocyclamino, haloheterocyclyl, carboxy ester, carboxy, carboxy amide, mercapto, alkylthio, benzylthio, acylthio and phosphorus-containing groups. As used herein, the term “optionally substituted” may also refer to the replacement of a CH₂ group with a carbonyl (C═O) group. Non-limiting examples of optional substituents include alkyl, preferably C₁₋₈ alkyl (e.g., C₁₋₆ alkyl such as methyl, ethyl, propyl, butyl, cyclopropyl, cyclobutyl, cyclopentyl or cyclohexyl), hydroxy C₁₋₈ alkyl (e.g., hydroxymethyl, hydroxyethyl, hydroxypropyl), alkoxyalkyl (e.g., methoxymethyl, methoxyethyl, methoxypropyl, ethoxymethyl, ethoxyethyl, ethoxypropyl etc.) C₁₋₈ alkoxy, (e.g., C₁₋₆ alkoxy such as methoxy, ethoxy, propoxy, butoxy, cyclopropoxy, cyclobutoxy), halo (fluoro, chloro, bromo, iodo), trifluoromethyl, trichloromethyl, tribromomethyl, hydroxy, phenyl (which itself may be further substituted, by an optional substituent as described herein, e.g., hydroxy, halo, methyl, ethyl, propyl, butyl, methoxy, ethoxy, acetoxy, amino), benzyl (wherein the CH₂ and/or phenyl group may be further substituted as described herein), phenoxy (wherein the CH₂ and/or phenyl group may be further substituted as described herein), benzyloxy (wherein the CH₂ and/or phenyl group may be further substituted as described herein), amino, C₁₋₈ alkylamino (e.g., C₁₋₆ alkyl, such as methylamino, ethylamino, propylamino), di C₁₋₈ alkylamino (e.g., C₁₋₆ alkyl, such as dimethylamino, diethylamino, dipropylamino), acylamino (e.g., NHC(O)CH₃), phenylamino (wherein phenyl itself may be further substituted as described herein), nitro, formyl, —C(O)—C₁₋₈, alkyl (e.g., C₁₋₆ alkyl, such as acetyl), O—C(O)-alkyl (e.g., C₁₋₆ alkyl, such as acetyloxy), benzoyl (wherein the CH₂ and/or phenyl group itself may be further substituted), replacement of CH₂ with C═O, CO₂H, CO₂C₁₋₈ alkyl (e.g., C₁₋₆ alkyl such as methyl ester, ethyl ester, propyl ester, butyl ester), CO₂phenyl (wherein phenyl itself may be further substituted), CONH₂, CONHphenyl (wherein phenyl itself may be further substituted as described herein), CONHbenzyl (wherein the CH₂ and/or phenyl group may be further substituted as described herein), CONH C₁₋₈ alkyl (e.g., C₁₋₆ alkyl such as methyl amide, ethyl amide, propyl amide, butyl amide), CONHdi C₁₋₈ alkyl (e.g., C₁₋₆alkyl).

“Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into stem cells that are committed to three, two or one hematopoietic differentiation pathway. None of these stem cells are morphologically distinguishable, however.

The term “hematopoietic stem cells” or “HSC” as used herein refers to multipotent stem cells that are capable of differentiating into all blood cells including erythrocytes, leukocytes and platelets. For instance, the term “hematopoietic stem cells” includes and encompasses those contained not only in bone marrow but also in umbilical cord blood derived cells.

The term “hematopoietic progenitors,” or “hematopoietic progenitor cells”, which are used interchangeably with the term “hematopoietic precursors,” refers to those progenitor or precursor cells which are differentiated further than hematopoietic stem cells but have yet to differentiate into progenitors or precursors of respective blood cell lineages (unipotent precursor cells). Thus, “progenitor cell(s)” or “precursor cell(s)” are defined as cells that are lineage-committed, i.e., an individual cell can give rise to progeny limited to a single lineage such as the myeloid or lymphoid lineage. They do not have self-renewal properties. They can also be stimulated by lineage-specific growth factors to proliferate. If activated to proliferate, progenitor cells have life-spans limited to 50-70 cell doublings before programmed cell senescence and death occurs. For example, “hematopoietic progenitors” as used herein include granulocyte/macrophage associated progenitors (colony-forming unit granulocyte, macrophage, CFU-GM), erythroid associated progenitors (burst-forming unit erythroid, BFU-E), megakaryocyte associated progenitors (colony-forming unit megakaryocyte, CFU-Mk), and myeloid associated stem cells (colony-forming unit mixed, CFU-Mix). Hematopoletic progenitor cells possess the ability to differentiate into a final cell type directly or indirectly through a particular developmental lineage. Undifferentiated, pluripotent progenitor cells that are not committed to any lineage are referred to herein as “stem cells.” All hematopoietic cells can in theory be derived from a single stem cell, which is also able to perpetuate the stem cell lineage, as daughter cells become differentiated. The isolation of populations of mammalian bone marrow cell populations which are enriched to a greater or lesser extent in pluripotent stem cells has been reported (see for example, C. Verfaillie et al., J. Exp. Med., 172, 509 (1990)).

“HSPC” as used herein refers to both hematopoietic progenitor cells and hematopoietic stem cells.

As used herein, “HIF-α potentiating agents” include agents that increase the accumulation of, or stability of, HIF-α; directly provide HIF-α activity; or increase expression of HIF-1. Such agents are known in the art, or may be identified through art-recognized screening methods. HIF-α potentiating agents include compounds that increase the accumulation and/or stability of HIF-α by inhibiting the activity of one or more HIF hydroxylase enzymes, e.g., one or more HIF prolyl hydroxylase enzymes. Inhibitors of HIF hydroxylase enzyme activity are well known, readily identified, and are further described herein.

“Homolog” is used herein to denote a gene or its product which is related to another gene or product by decent from a common ancestral DNA sequence.

As used herein, the term “hyperproliferative cell disorder” refers to a disorder in which cellular hyperproliferation causes or contributes to the pathological state or symptoms of the disorder. Illustrative hyperproliferative cell disorders include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. Exemplary hyperproliferative cell disorders include: cancers; blood vessel proliferative disorders such as restenosis, atherosclerosis, in-stent stenosis, vascular graft restenosis, etc.; fibrotic disorders; psoriasis; inflammatory disorders, e.g., arthritis, etc.; glomerular nephritis; endometriosis; macular degenerative disorders; benign growth disorders such as prostate enlargement and lipomas; autoimmune disorders; and scarring disorders such as post-operative scarring, hypertrophic scarring, keloid scarring and glial scarring. In some embodiments, the hyperproliferative cell disorder is a precancer or a precancerous condition. A “precancer cell” or “precancerous cell” is a cell manifesting a hyperproliferative cell disorder that is a precancer or a precancerous condition. In other embodiments, the hyperproliferative cell disorder is a cancer. The term “cancer” includes primary and metastatic cancer and is used interchangeably herein with the term “neoplastic” to refer to a disease or condition involving cells that metastasize or have the potential to metastasize to distal sites and exhibit phenotypic traits that differ from those of non-neoplastic cells, for example, formation of colonies in a three-dimensional substrate such as soft agar or the formation of tubular networks or web-like matrices in a three-dimensional basement membrane or extracellular matrix preparation, such as Matrigel™. Non-neoplastic cells do not form colonies in soft agar and form distinct sphere-like structures in three-dimensional basement membrane or extracellular matrix preparations. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells. Exemplary cancers includes solid tumors, as well as, hematologic tumors and/or malignancies. A “cancer cell,” “cancerous cell” or “neoplastic cell” is a cell manifesting a hyperproliferative cell disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers. In some embodiments, the hyperproliferative cell disorder is a non-neoplastic disorder in which cellular hyperproliferation causes or contributes to the pathological state or symptoms of the disorder.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The phrase “hybridizing specifically to” and the like refer to the binding, duplexing, or hybridizing of a molecule only to a particular nucleotide sequence under stringent conditions when that sequence is present in a complex mixture (e.g., total cellular) DNA or RNA.

“Hypoxia-inducible factor α” or “HIF-α” is a subunit of the oxygen-dependent transcriptional activator, hypoxia-inducible factor (HIF), which plays crucial roles in the angiogenesis of tumors and mammalian development. HIF is a heterodimeric protein consisting of a constitutively expressed HIF-1β subunit and one of three subunits HIF-α (HIF-1α, HIF-2α or HIF-3α). The most widely studied, and seemingly major, HIF isoform is the HIF-1 isoform. The stability and activity of HIF-α subunits are regulated by various post-translational modifications, including hydroxylation, acetylation, and phosphorylation. Under normoxia, the HIF-α subunit is rapidly degraded via the von Hippel-Lindau tumor suppressor gene product (vHL)-mediated ubiquitin-proteasome pathway. The association of vHL and HIF-α under normoxic conditions is triggered by the hydroxylation of prolines and the acetylation of lysine within a polypeptide segment known as the oxygen-dependent degradation domain (ODDD). The hydroxylation of proline residues (e.g., specifically proline 402 and 564 in human HIF-1α polypeptide) within the ODDD is carried out by specific HIF-prolyl hydroxylases (HPHI-3 also referred to as PHD1-3) in the presence of iron, oxygen, and 2-oxoglutarate. During hypoxic conditions HIF-α subunit becomes stable and interacts with co-activators such as p300/CBP to modulate its transcriptional activity. HIF-1 acts as a master regulator of numerous hypoxia-inducible genes under hypoxic conditions. The heterodimer HIF-1 binds to the hypoxic response elements (HREs) of target gene regulatory sequences, resulting in the transcription of genes implicated in the control of cell proliferation/survival, glucose/iron metabolism and angiogenesis, as well as apoptosis and cellular stress. Some of these direct target genes include glucose transporters, the glycolytic enzymes, erythropoietin, and angiogenic factor vascular endothelial growth factor (VEGF). “HIF-α” is the oxygen-responsive component of HIF-1 and may refer to any mammalian or non-mammalian HIF-α polypeptide or fragment thereof, e.g., HIF-1 α, HIF-2α, or HIF-3 α. In specific embodiments, the term refers to the human form of HIF-1α, as set forth for example in GenPept Accession Nos. AAC50152, NP_{—}001521, NP_{—}851397 and NP_{—}001230013. HIF-α coding or gene sequences are also encompassed, as discussed for example infra. A fragment of HIF-α of interest is any fragment retaining at least one functional or structural characteristic of HIF-α. Non-limiting fragments of HIF-1α suitably include proline residue 402 and/or 564 (as set forth in GenPept Accession No. AAC50152), which are hydroxylated by PHD polypeptides. Suitable fragments may include or consist of residues 344-698, particularly residues 364-678, more particularly residues 364-638 or 384-638 and still more particularly residues 364-598 or 394-598. Other suitable fragments may include or consist of residues 549-652 and even more particularly the N-terminal region thereof which interacts with the vHL protein. C-terminal fragments may include residues 549 to 582 and in particular residues 556-574. Other suitable fragments comprise or consist of residues 344-417, more suitably 380-417. Such a region, or its equivalent in other HIF-α subunit proteins, is desirably present in HIF-α substrates used in assays for screening PHD inhibitors. Exemplary HIF-α fragments may typically comprise residues 549 to 582 of the human HIF-1α sequence.

The term “immunocompromised” as used herein refers to a subject with an innate, acquired, or induced inability to develop a normal immune response. An immunocompromised subject, therefore, has a weakened or impaired immune system relative to one of a normal subject. A subject with a weakened or impaired immune system has an “immunodeficiency” or “immunocompromised condition,” which is associated with a primary or secondary deficiency, induced or non-induced, in one or more of the elements of the normal immune defense system. An immunocompromised condition is commonly due to a medical treatment, e.g., radiation therapy, chemotherapy or other immunosuppressing treatment, such as induced by treatment with steroids, cyclophosphamide, azathioprine, methotrexate, cyclosporine or rapamycin, in particular in relation to cancer treatment or the treatment or prevention of transplant rejection. However, it will be understood that the phrase “risk of acquiring an immunocompromised condition resulting from a medical treatment” refers only to medical treatments that leads to or confers an immunocompromised condition, especially chemotherapy or other immunosuppressing treatment, such as induced by treatment with radiation, steroids, cyclophosphamide, azathioprine, methotrexate, cyclosporine or rapamycin. The presence of an immunocompromised condition in a subject can be diagnosed by any suitable technique known to persons of skill the art. Strong indicators that an immunocompromised condition may be present is when rare diseases occur or the subject gets ill from organisms that do not normally cause diseases, especially if the subject gets repeatedly infected. Other possibilities are typically considered, such as recently acquired infections—for example, HIV, hepatitis, tuberculosis, etc. Generally, however, definitive diagnoses are based on laboratory tests that determine the exact nature of the immunocompromised condition. Most tests are performed on blood samples. Blood contains antibodies, lymphocytes, phagocytes, and complement components—all of the major immune components that might cause immunodeficiency. A blood cell count will determine if the number of phagocytic cells or lymphocytes is below normal. Lower than normal counts of either of these two cell types correlates with an immunocompromised condition. The blood cells are also checked for their appearance. Occasionally, a subject may have normal cell counts, but the cells are structurally defective. If the lymphocyte cell count is low, further testing is usually conducted to determine whether any particular type of lymphocyte is lower than normal. A lymphocyte proliferation test may be conducted to determine if the lymphocytes can respond to stimuli. The failure to respond to stimulants correlates with an immunocompromised condition. Antibody levels and complement levels can also be determined for diagnosing the presence of an immunocompromised condition. However, it shall be understood that the methods of the present invention are not predicated upon diagnosing the absence of an immunocompromised condition in the subjects to be treated.

Reference herein to “immuno-interactive” includes reference to any interaction, reaction, or other form of association between molecules and in particular where one of the molecules is, or mimics, a component of the immune system.

Reference herein to an “infectious agent,” “infectious organism,” “microbe” or “pathogen” includes any one or more species or subspecies of bacterium, fungus, virus, algae, parasite, (including ecto- or endo-parasites) prion, oomycetes, slime, moulds, nematodes, mycoplasma and the like. The present invention is particularly suited to treating or preventing mixed infections by more than one microbe. Pathogenic algae include Prototheca and Pfiesteria. Also includes within the scope of these terms are prion proteins causing conditions such as Creutzfeldt-Jakob disease. As the skilled artisan will appreciate, pathogenicity or the ability of a classically non-pathogenic agent to infect a subject and cause pathology can vary with the genotype and expression profile of the infectious agent, the host and the environment. Fungal pathogens include without limitation species of the following genera: Absidia, Acremonium, Aspergillus, Basidiobolus, Bipolaris, Blastomyces, Candida (yeast), Cladophialophora, Coccldioides, Cryptococcus, Cunninghamella, Cuwvularia, Epidermophyton, Exophiala, Erserohilum, Fonsecaea, Fusarium, Geotrichum, Histoplasma, Hortaea, Lacazia, Lasiodiplodia, Leptosphaeria, MadureUlla, Malassezia, Microsporum, Mucor, Neotestudina, Onychocola, Paecilomyces, Paracoccidioides, Penicillfum, Phlalophora, Piedraia, Piedra, Pityriasis, Pneumocystis, Pseudallescheria, Pyrenochaeta, Rhizomucor, Rhizopus, Rhodotorula, Scedosporium, Scopulariopsis, Scytalidium, Sporothrix, Trichophyton, Trichosporon and Zygomycete. Pathogenic conditions include any deleterious condition that develops as a result of infection with an infectious organism.

As used herein, the term “interact” includes close contact between molecules that results in a measurable effect, e.g., the binding or association of one molecule to another or a reaction of one molecule with another.

By “isolated” is meant material that is substantially or essentially free from components that normally accompany it in its native state.

The term “lower alkyl” refers to straight and branched chain alkyl groups having from 1 to 6 carbon atoms, such as methyl, ethyl, n-propyl, iso-propyl, n-butyl, tert-butyl, sec-butyl, n-pentyl, n-hexyl, 2-methylpentyl, and the like. In some embodiments, the lower alkyl group is methyl or ethyl.

The term “lower alkoxy” refers to straight and branched chain alkoxy groups having from 1 to 6 carbon atoms, such as methoxy, ethoxy, n-propoxy, iso-propoxy, n-butoxy, tert-butoxy, sec-butoxy, n-pentoxy, n-hexoxy, 2-methyl-pentoxy, and the like. Usually, the lower alkoxy group is methoxy or ethoxy.

As used herein, a “mobilizer of hematopoietic stem cells and/or progenitor cells,” “mobilizing agent” or “mobilizer” are used interchangeably to refer to any compound, whether it is a small organic molecule, synthetic or naturally derived, or a polypeptide, such as a growth factor or colony stimulating factor or an active fragment or mimic thereof, a nucleic acid, a carbohydrate, an antibody, or any other agent that acts to enhance the migration of stem cells from the bone marrow into the peripheral blood. Such a “mobilizer” may increase the number of hematopoietic stem cells or hematopoietic progenitor/precursor cells in the peripheral blood.

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

A “neutropenia medicament” as used herein refers to a composition of matter which reduces the symptoms related to neutropenia, prevents the development of neutropenia, or treats existing neutropenia.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The term “operably connected” or “operably linked” as used herein refers to a juxtaposition wherein the components so described are in a relationship permitting them to function in their intended manner. For example, a transcriptional control sequence “operably linked” to a coding sequence or non-coding sequence refers to positioning and/or orientation of the transcriptional control sequence relative to the coding or non-coding sequence to permit expression of the coding or non-coding sequence under conditions compatible with the transcriptional control sequence.

The term “pharmaceutically acceptable” as used herein refers to a compound or combination of compounds that will not impair the physiology of the recipient human or animal to the extent that the viability of the recipient is compromised. Suitably, the administered compound or combination of compounds will elicit, at most, a temporary detrimental effect on the health of the recipient human or animal.

By “pharmaceutically acceptable carrier” is meant a pharmaceutical vehicle or solvent comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like. Illustrative vehicles or solvents include without limitation water, saline, physiological saline, ointments, creams, oil-water emulsions, gels, or any other vehicle/solvent or combination of vehicles/solvents and compounds known to one of skill in the art that is pharmaceutically and physiologically acceptable to the recipient human or animal.

Similarly, a “pharmacologically acceptable” salt, ester, amide, prodrug or derivative of a compound as provided herein is a salt, ester, amide, prodrug or derivative that this not biologically or otherwise undesirable.

Pathogenic “protozoa” include, without limitation, Trypanosoma, Leshmania, Giardia, Trichomonas, Ennamoeba, Naegleria, Acanthamoeba, Plasmodium, Toxoplasma, Cryptosporidium, Isospora and Balantidium.

Larger pathogenic “parasites” include those from the phyla Cestoda (tapeworms), Nematoda and Trematoda (flukes). Pathogenic trematodes are, for example, species of the following genera; Schistosoma, Echinostoma, Fasciolopsis, Clonorchis, Fasciola, Opisthorchis and Paragonimus. Cestode pathogens include, without limitation, species from the following orders; Pseudophyllidea (e.g., Diphyllobothrium) and Cyclophyllidea (e.g., Taenia). Pathogenic nematodes include species from the orders; Rhabditida (e.g., Strongyloides), Strongylida (e.g., Ancylostoma), Ascaridia (e.g., Ascaris, Toxocara), Spirurida (e.g., Dracunculus, Brugia, Onchocerca, Wucheria) and Adenophorea (e.g., Trichuris and Trichinella).

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The terms “polynucleotide variant” and “variant” refer to polynucleotides displaying substantial sequence identity with a reference polynucleotide sequence or polynucleotides that hybridize with a reference sequence under stringent conditions as known in the art (see for example Sambrook et al., Molecular Cloning. A Laboratory Manual”, Cold Spring Harbor Press, 1989). These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally-occurring allelic variants.

The terms “polypeptide,” “proteinaceous molecule,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. These terms also encompass polypeptides in which one or more amino acids have been added or deleted, or replaced with different amino acids.

As used herein, the terms “prevent,” “prevented,” or “preventing,” when used with respect to the treatment of an immunocompromised condition (e.g., anemia, thrombocytopenia, agranulocytosis or neutropenia), refers to a prophylactic treatment which increases the resistance of a subject to developing the immunocompromised condition or, in other words, decreases the likelihood that the subject will develop the immunocompromised condition as well as a treatment after the immunocompromised condition has begun in order to reduce or eliminate it altogether or prevent it from becoming worse.

The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester derivative.

As used herein, “racemate” refers to a mixture of enantiomers.

As used herein; a “reporter gene” refers to any gene or DNA that expresses a product that is detectable by spectroscopic, photochemical, biochemical, enzymatic, immunochemical, electrical, optical or chemical means. The preferred reporter gene to which a promoter element is ligated is luciferase. Other reporter genes for use for this purpose include, for example, β-galactosidase gene (β-gal) and chloramphenicol acetyltransferase gene (CAT) Assays for expression produced in conjunction with each of these reporter gene elements are well-known to those skilled in the art.

The terms “salts,” “derivatives” and “prodrugs” includes any pharmaceutically acceptable salt, ester, hydrate, or any other compound which, upon administration to the recipient, is capable of providing (directly or indirectly) a compound of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicyclic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl, and butyl chlorides, bromides and iodides; dialkyl sulfates like dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art. For example, metal salts can be prepared by reaction of a compound of the invention with a metal hydroxide. An acid salt can be prepared by reacting an appropriate acid with a compound of the invention.

The term “selective” refers to compounds that inhibit or display antagonism towards a PHD (e.g., PHD1, PHD2, or PHD3) (e.g., a prolyl-4-hydroxylase) without displaying substantial inhibition or antagonism towards another PHD. Accordingly, a compound that is selective for a particular PHD (e.g., a prolyl-4-hydroxylase) exhibits inhibition or antagonism of that PHD that is greater than about 2-fold, 5-fold, 10-fold, 20-fold, 50-fold or greater than about 100-fold with respect to inhibition or antagonism of another PHD. In some embodiments, selective compounds display at least 50-fold greater inhibition or antagonism towards a particular PHD (e.g., a prolyl-4-hydroxylase) than towards another PHD. In still other embodiments, selective compounds inhibit or display at least 100-fold greater inhibition or antagonism towards a particular PHD (e.g., a prolyl-4-hydroxylase) than towards another PHD. In still other embodiments, selective compounds display at least 500-fold greater inhibition or antagonism towards a particular PHD (e.g., a prolyl-4-hydroxylase) than towards another PHD. In still other embodiments, selective compounds display at least 1000-fold greater inhibition or antagonism towards a particular PHD (e.g., a prolyl-4-hydroxylase) than towards another PHD

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (I.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table I below.

TABLE 1

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP. Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As used herein a “small molecule” refers to a composition that has a molecular weight of less than 3 kilodaltons (kDa), and typically less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, or even less than about 1 kDa.

“Stem cells” refer to cells, which are not terminally differentiated and are therefore able to produce cells of other types. Stem cells are generally divided into three types, including totipotent, pluripotent, and multipotent. “Totipotent stem cells” can grow and differentiate into any cell in the body, and thus can grow into an entire organism. These cells are not capable of self-renewal. In mammals, only the zygote and early embryonic cells are totipotent. “Pluripotent stem cells” are true stem cells, with the potential to make any differentiated cell in the body, but cannot contribute to making the extraembryonic membranes (which are derived from the trophoblast). “Multipotent stem cells” are clonal cells that self-renew as well as differentiate to regenerate adult tissues. “Multipotent stem cells” are also referred to as “unipotent” and can only become particular types of cells, such as blood cells or bone cells. The term “stem cells”, as used herein, refers to pluripotent stem cells capable of self-renewal.

“Stringency” as used herein refers to the temperature and ionic strength conditions, and presence or absence of certain organic solvents, during hybridization. The higher the stringency, the higher will be the observed degree of complementarity between sequences. “Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization, and is greatly changed when nucleotide analogues are used. Generally, stringent conditions are selected to be about 10° C. to 20° C. less than the thermal melting point (Tm) for the specific sequence at a defined ionic strength and pH. The Tm is the temperature (under defined ionic strength and pH) at which 50% of a target sequence hybridizes to a complementary probe. It will be understood that a polynucleotide will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

“Subjects” contemplated in the present invention include any animal of commercial, humanitarian, or epidemiological interest including conveniently, primates, livestock animals (such as sheep, cows, horses, donkeys, pigs, fish and birds), laboratory test animals (such as mice, rabbits, guinea pigs and hamsters and the like), companion animals (such as dogs and cats), or captive wild animals. Avian species include poultry birds and caged avian species. In some embodiments the subject is a mammalian animal. In other embodiments, the subject is a human subject. The present composition and methods have applications in human and veterinary medicine, domestic or wild animal husbandry, cosmetic or aesthetic treatments for the skin after injury or surgery. A donor subject is the subject in which the mobilization of hematopoietic stem and/or progenitor cells occurs and/or from which the mobilized stem and/or progenitor cells are harvested, if any. A recipient subject is the subject to which the harvested stem and/or progenitor cells are transplanted. The donor subject and the recipient subject may be the same subject or may be different subjects.

By “substantially complementary” it is meant that an oligonucleotide or a subsequence thereof is sufficiently complementary to hybridize with a target sequence. Accordingly, the nucleotide sequence of the oligonucleotide or subsequence need not reflect the exact complementary sequence of the target sequence. In a preferred embodiment, the oligonucleotide contains no mismatches and with the target sequence.

As used herein, the term “synergistic” means that the therapeutic effect of a HIF-α potentiating agent when administered in combination with at least one mobilizer of hematopoietic stem cells and/or progenitor cells (or vice-versa) is greater than the predicted additive therapeutic effects of the HIF-α potentiating agent and the at least one mobilizer when administered alone. The term “synergistically effective amount” as applied to a HIF-α potentiating agent and at least one mobilizer of hematopoietic stem cells and/or progenitor cells refers to the amount of each component in a composition (generally a pharmaceutical composition), which is effective for stimulating or enhancing mobilization of hematopoietic stem cells and/or progenitor cells from the bone marrow into the peripheral blood, and which produces an effect which does not intersect, in a dose-response plot of the dose of HIF-α potentiating agent versus a dose of the at least one mobilizer versus stimulating or enhancing mobilization of hematopoietic stem cells and/or progenitor cells from the bone marrow into the peripheral blood, either the dose HIF-α potentiating agent axis or the dose at least one mobilizer axis. The dose response curve used to determine synergy in the art is described for example by Sande et al. (see, p. 1080-1105 in A. Goodman et al., ed., the Pharmacological Basis of Therapeutics, MacMillan Publishing Co., Inc., New York (1980)). The optimum synergistic amounts can be determined, using a 95% confidence limit, by varying factors such as dose level, schedule and response, and using a computer-generated model that generates isobolograms from the dose response curves for various combinations of the HIF-α potentiating agent and the at least one mobilizer. The highest mobilization of hematopoietic stem cells and/or progenitor cells on the dose response curve correlates with the optimum dosage levels. A “thrombocytopenia medicament” as used herein refers to a composition of matter which reduces the symptoms related to thrombocytopenia, prevents the development of thrombocytopenia, or treats existing thrombocytopenia.

As used herein, the term “transcriptional control sequence” refers to nucleic acid sequences, such as initiator sequences, enhancer sequences and promoter sequences, which induce, repress, or otherwise control the transcription of protein encoding nucleic acid sequences to which they are operably-linked.

By “treatment,” “treat,” “treated,” “treating” and the like is meant to include both therapeutic and prophylactic treatment, including the administration of medicine or the performance of medical procedures with respect to a patient, for either prophylaxis (prevention) or to cure or reduce the extent of or likelihood of occurrence of the infirmity or malady or condition or event in the instance where the patient is afflicted. In some embodiments of the present invention, the treatments using the agents described may be provided to treat patients suffering from a hyperproliferative cell disorder, whereby the treatment of the disorder with a cytoreductive or myeloablative therapy (e.g., chemotherapy or radiation therapy) results in a decrease in bone marrow cellularity, thus making the patient more immunocompromised and more prone therefore to acquiring infectious agents or diseases. Thus, the administration of the agents of the invention allows for enhanced mobilization of hematopoietic stem cells and/or progenitor cells from the bone marrow to the peripheral blood. In some embodiments, the treating is for the purpose of reducing or diminishing the symptoms or progression of a hyperproliferative cell disorder by allowing for the use of accelerated doses of chemotherapy or radiation therapy.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

Reference herein to “a virus” includes any virus or viral pathogen or emerging viral pathogen. Viral families contemplated include Adenoviridae, African swine fever-like viruses, Arenaviridae (such as viral hemorrhagic fevers, Lassa fever), Astroviridae (astroviruses) Bunyaviridae (La Crosse), Caliciviridae (Norovirus), Coronaviridae (Corona virus), Filoviridae (such as Ebola virus, Marburg virus), Parvoviridae (B19 virus), Flaviviridae (such as hepatitis C virus, Dengue viruses), Hepadnaviridae (such as hepatitis B virus, Deltavirus), Herpesviridae (herpes simplex virus, varicella zoster virus), Orthomyxoviridae (influenza virus) Papovaviridae (papilloma virus) Paramyxoviridae (such as human parainfluenza viruses, mumps virus, measles virus, human respiratory syncytial virus, Nipah virus, Hendra virus), Picornaviridae (common cold virus), Poxviridae (small pox virus, orf virus, monkey poxvirus) Reoviridae (rotavirus) Retroviridae (human immunodeficiency virus) Parvoviridae (parvoviruses) Papillomaviridae, (papillomaviruses) alphaviruses and Rhabdoviridae (rabies virus).

As used herein, underscoring or italicizing the name of a gene shall indicate the gene, in contrast to its protein product, which is indicated by the name of the gene in the absence of any underscoring or italicizing. For example, “HIF-1α” shall mean the HIF-1α gene, whereas “HIF-1α” shall indicate the protein product or products generated from transcription and translation and alternative splicing of the “HIF-1α” gene.

Each embodiment described herein is to be applied mutatis mutandis to each and every embodiment unless specifically stated otherwise.

### 2. Abbreviations

CFC=colony-forming cells

HIF=hypoxia-inducible factor

HIFα=hypoxia-inducible factor-α

HSC=hematopoietic stem cells

HSPC=hematopoietic stem and progenitor cells

d=day

h=hour

s=seconds

i.v.=intravenous

i.p.=intraperitoneal

rHu=recombinant human

s.c.=subcutaneous

### 3. Compositions and Methods for Enhancing Hematopoietic Function

The present invention is based in part on the surprising discovery that mobilization of hematopoietic stem cells and/or progenitor cells by mobilizing agents such as G-CSF and Plerixafor, and by combinations thereof, is significantly enhanced in the presence of HIF-α potentiating agents. This increased mobilization in turn results in higher numbers of hematopoietic stem and progenitor cells (HSPCs) migrating from the bone marrow into the peripheral blood when compared to the number resulting from administration of stem cell mobilizers alone. The increased mobilization may also result in increased HSPCs mobilizing from the peripheral blood to particular tissues or organs such as the lymph nodes, the heart, the lung, the liver, the skin, the spleen, small and large intestines, the stomach, or the pancreas.

Increasing the number or mobility of HSPCs may also increase the rate of differentiation of HSPCs into various cell lineages. The HSPCs may also be capable of differentiation or starting a path to becoming a mature hematopoietic cell. For example, the differentiation of the HSPCs may lead to an increase in the number of common myeloid progenitor cells in the bone marrow or the peripheral blood. The differentiation of HSPCs may also lead to an increase in the number of granulocyte/macrophage progenitor cells or megakaryocyte/erythrocyte progenitor cells in the bone marrow or peripheral blood. The HSPCs may differentiate into a common lymphoid precursor. The increase in number of common myeloid progenitor cells may lead to a differentiation into granulocyte/macrophage progenitor cells or megakaryocyte/erythrocyte progenitor cells. The granulocyte/macrophage progenitor cells may further differentiate into granulocytes such as neutrophils, eosinophils, basophils, tissue precursor cells, monocytes, and immature dendritic cells. The megakaryocyte/erythrocyte progenitor cells may differentiate into megakaryocytes and erythroblasts. The common lymphoid precursor cell may differentiate into B lymphocyte cells and T lymphocyte cells. The B lymphocyte cells may differentiate into antibody-secreting cells, wherein T lymphocytes may differentiate into effector T cells. The granulocyte may further differentiate into tissue mast cells, macrophages, and immature dendritic cells. The megakaryocyte may differentiate into platelets. The erythroblast may differentiate into erythrocytes. HSPCs may also be capable of differentiating into cells such as muscle (skeletal myocytes and cardiomyocytes), brain, liver, skin, lung, kidney, intestinal, and pancreatic. The number or proportion of cells presenting particular molecular or cell surface markers may be indicative of an HSPC or HSPC population.

Although not wishing to be bound by any theory or mode of operation, 1 in every 10,000 to 15,000 bone marrow cells may normally be a stem cell. In the bloodstream, the proportion may fall to 1 to 100,000 blood cells. Administering a HIF-α potentiating agent and at least one mobilizer of hematopoietic stem cells and/or progenitor cells in vivo may increase the number of all stem cell populations in the bloodstream in about 1 hr or less, 2 hrs or less, 3 hrs or less, 4 hrs or less, 6 hrs or less, 8 hrs or less, 10 hrs or less, 12 hrs or less, 14 hrs or less, 16 hrs or less, 18 hrs or less, 20 hrs or less, 22 hrs or less, 24 hrs or less, 26 hrs or less, 28 hrs or less, or 30 hours or less after administration, and accumulation of stem cells including HSPC in the blood may peak in about 65 hrs or less, 66 hrs or less, 67 hrs or less, 68 hrs or less, 69 hrs or less, 70 hrs or less, 71 hrs or less, 72 hrs or less, 73 hrs or less, 74 hrs or less, 75 hrs or less, 76 hrs or less, 77 his or less, 78 hrs or less, 79 hrs or less, 80 hrs or less, 81 hrs or less, 82 his or less, 83 hrs or less, 84 hrs or less, 85 hrs or less, 86 hrs or less, 87 hrs or less, 88 hrs or less, 89 hrs or less, 90 hrs or less, 91 hrs or less, 92 hrs or less, 93 hrs or less, 94 hrs or less, 95 hrs or less, 96 hrs or less, 97 hrs or less, 98 hrs or less, 99 hrs or less, 100 hrs or less, 101 hrs or less, 102 hrs or less, 103 hrs or less, 104 hrs or less, 105 hrs or less, 106 hrs or less, 107 hrs or less, 108 hrs or less, 109 his or less, and 110 hrs or less after administration.

Thus, in accordance with the present invention, methods and compositions are provided that take advantage of a HIF-α potentiating agent and at least one mobilizer of hematopoietic stem cells and/or progenitor cells for stimulating or enhancing mobilization of hematopoietic stem cells and/or progenitor cells from the bone marrow into the peripheral blood, for stimulating or enhancing hematopoiesis, for the treatment or prophylaxis of immunocompromised conditions, including ones resulting from medical treatments that target rapidly dividing cells or that disrupt the cell cycle or cell division (e.g., myeloablative therapy), or for stem cell transplantation.

3.1 HIF-α Potentiating Agents

The HIF-α potentiating agent includes and encompasses any active agent that increases the accumulation of, or stability of, HIF-α; directly provides HIF-α activity; or increases expression of HIF-α, including without limitation, small molecules and macromolecules such as nucleic acids, peptides, polypeptides, peptidomimetics, carbohydrates, polysaccharides, lipopolysaccharides, lipids or other organic (carbon containing) or inorganic molecules. HIF-α refers to one or more of HIF-1α, HIF-2α HIF-3α.

Thus, in some embodiments, the HIF-α potentiating agent can be anything that results in an increase in the amount or activity of a HIF-α polypeptide. Non-limiting examples include agents: that improve the stability (e.g., half-life) of the protein; that block or reduce deactivation of the HIF-α polypeptide, for example by preventing the hydroxylation and/or acetylation; and agents that increase the amount of HIF-α polypeptide in a sample under consideration, for example by increasing the amount expressed from a HIF-α coding sequence or by introducing multiple copies of a HIF-α coding sequence. Accordingly, without limiting the present invention to any particular mechanism, with regard to an exemplary mode of action, the HIF-α potentiating agent can increase the activity of the HIF-α polypeptide by acting directly or indirectly on the HIF-α polypeptide to stabilize the protein, protect it from inhibition, or to increase the activity of the protein. Alternatively, the substance can increase the activity of the HIF-α polypeptide by inhibiting or otherwise blocking the activity of compounds or enzymes that inhibit the activity or reduce the stability of the HIF-α polypeptide

In certain embodiments, the method includes introducing into at least one cell of the subject, such as a hematopoietic stem cell or progenitor cell, a nucleic acid construct that comprises at least one HIF-α coding sequence operable connected to a transcriptional control sequence, and permitting the cell to express the encoded HIF-α polypeptide. Non-limiting examples of HIF-α coding sequences include: human HIF-1α coding sequences as disclosed for example in GenBank Accession Nos. NM001530, Q9NWT60, U22431, AB073325, AF208487 and AF304431; bovine HIF-α coding sequences as disclosed for example in GenBank Accession Nos. Q9XTA5, AB018398 and BAA78675; rat HIF-α coding sequences as disclosed for example in GenBank Accession Nos. AF057308, 035800 and CAA70701; mouse HIF-α coding sequences as disclosed for example in GenBank Accession Nos. AF003695, AAC52730, AF057308 and Q61221; squirrel HIF-α coding sequences as disclosed for example in GenBank Accession No. AY713478; avian HIF-α coding sequences as disclosed for example in GenBank Accession No. Q9YIB9; amphibian HIF-α coding sequences as disclosed for example in GenBank Accession No. Q98SW2; antelope HIF-α coding sequences as disclosed for example in GenBank Accession No. AY971808; Xenopus laevis HIF-α coding sequences as disclosed for example in GenBank Accession No. CAB96628; Drosophila melanogaster HIF-α coding sequences as disclosed for example in GenBank Accession No. JC485 1; zebra fish HIF-α coding sequences as disclosed for example in GenBank Accession No. AY326951; chicken HIF-α coding sequences as disclosed for example in GenBank Accession Nos. ABA02179 and BAA34234, and the like. Others species of interest would be dogs, cats, and other domesticated and farm animals, such as pigs and horses. HIF-α may also be any mammalian or non-mammalian protein or fragment thereof. HIF-α gene sequences may also be obtained by routine cloning techniques, for example by using all or part of a HIF-α gene sequence described above as a probe to recover and determine the sequence of a HIF-α gene in another species. A fragment of HIF-α of interest is any fragment retaining at least one functional or structural characteristic of HIF-α. Fragments of HIF-α include, e.g., the regions defined by human HIF-α from amino acids 401 to 603 (Huang et al., (1998) Proc Natl Acad Sci. USA 95:7987-7992), amino acid 531 to 575: (Jiang et al. (1997) J Biol Chem. 272:19253-19260), amino acid 556 to 575 (Tanimoto et al. (2000) EMBO J. 19:4298-4309), amino acid 557 to 571 (Srinivas et al. (1999) Biochem Biophys Res Commun. 260:557-561), and amino acid 556 to 575 (Ivan and Kaelin (2001) Science 292:464-468). Further, HIF-α fragments include any fragment containing at least one occurrence of the motif LXXLAP, e.g., as occurs in the human HIF-α native sequence at L₃₉₇TLLAP and L₅₅₉EMLAP.

In other embodiments, HIF-α potentiating agents stimulate or enhance expression of HIF-α, representative examples of which include metallothionein and zinc (see, e.g., Xue et al. (2012) Am J Physiol Heart Circ Physiol 302: H2528-H2535).

In some embodiments, HIF-α potentiating agents inhibit the level or activity of a HIF-α interacting protein that inhibits the activity of a HIF-α polypeptide. Non-limiting HIF-1 interacting proteins of this type include: the von Hippel-Lindau tumor suppressor protein (vHL, Hon et al. (2002) Nature 417:975-8; Min et al. (2002) Science 296:1886-9); hydroxylases including prolyl hydroxylases (e.g., proly-4-hydroxylases) (also referred herein as HIF hydroxylases such as the HIF prolyl hydroxylases PHD1, PHD2 and PHD3, as described for example by Epstein et at (2001) Cell 107:43-54, Kaelin (2005) Annu Rev Biochem. 74:115-28; Schmid et al. (2004) J Cell Mol Med., 8:423-31; Huang et al. (2002) J Biol Chem. 277:39792-800; and Metzen et al. (2003) J Cell Si. 116:1319-26), dehydroxylases, ubiquitylation and deubiquitylation enzymes, ARDI acetyltransferase (as described for example by Jeong et al. (2002) Cell 111:709-20), factor inhibiting HIF-1 (FIH-1; as described for example by Hewitson et al. (2002) J Biol Chem. 277(29):26351-5; Lando et al. (2002) Genes Dev. 16:1466-71; PCT Application Publication Nos. WO03028663, WO04035812, WO02074981); inhibitory PAS domain protein (IPAS, Makino et al. (2002) Nature 414:550-4) and the like, which interact with one or more proteins comprising the HIF-1 heterodimer and/or modulate the activity thereof. Of particular interest are human HIF-α interacting proteins (see, e.g., Accession Nos. P40337, NP_{—}000542, NP937799, NP005154, NP060372, NP003363, and the like) and homologues, analogues and isoforms thereof (including animal homologues). Those of skill in the art will readily be able to identify additional HIF-α interacting proteins suitable in the present invention.

Thus, in the HIF-α pathway in which the HIF-α polypeptide interacts with several HIF-α interacting proteins, there are several possible points of therapeutic intervention. First, HIF-α activity or protein levels can be increased by using small molecules to disrupt the rapid degradation of HIF-α (Hewitson, K S and Schofield, C J. (2004) Drug Discovery Today 9(16):704-711). This would include, for example, inhibitors of PHD1-3 (prolyl hydroxylase domain-containing enzymes 1-3), which include prolyl-4-hydroxylase inhibitors, illustrative examples of which include oxalamic acid alkyl esters (e.g., dimethyloxallyl glycine) and disubstituted pyridines (e.g., diethylpyridine dicarboxylate); inhibitors of FIH (factor inhibiting HIF) such as, for example, dihydrobenzoic acids (e.g., 3,4-dihydrobenzoate); proteasomal inhibitors that affect degradation of the HIF-α subunit; small molecules or antibodies that would block vHL complex:HIF-α interaction; small molecule inhibitors of ubiquitination; and inhibitory nucleic acid molecules such as small interfering RNAs (siRNAs) targeting PHD1-3 and/or FIH.

In some embodiments, the HIF-α potentiating agent is an antagonistic nucleic acid molecule that functions to inhibit the transcription or translation of PHD, FIH-1 or vHL encoding transcripts. Representative transcripts of this type include nucleotide sequences corresponding to any one the following sequences: (1) human PHD) nucleotide sequences as set forth for example in GenBank Accession Nos. AJ310544, BC036051, NM_{—}053046 and NM_{—}080732; human PHD2 nucleotide sequences as set forth for example in GenBank Accession Nos. NM_{—}022051 and NG_{—}015865; human PHD3 nucleotide sequences as set forth for example in GenBank Accession Nos. NM_{—}022073 and AJ310545; human FIH-1 nucleotide sequences as set forth for example in GenBank Accession No. NM_{—}017902; and human vHL nucleotide sequences as set forth for example in GenBank Accession Nos. NM_{—}000551 and NM_{—}198156; (2) nucleotide sequences that share at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) nucleotide sequences that hybridize under at least low, medium or high stringency conditions to the sequences referred to in (1); (4) nucleotide sequences that encode any one of the following amino acid sequences: human PHD1 amino acid sequences as set forth for example in GenPept Accession Nos. CAC42510, AAH36051, NP_{—}444274 and NP_{—}542770; human PHD2 amino acid sequences as set forth for example in GenPept Accession Nos. NP_{—}071334 and NP_{—}071334; human PHD3 amino acid sequences as set forth for example in GenPept Accession Nos. NP_{—}071356 and CAC42511; human FIH-1 amino acid sequences as set forth for example in GenPept Accession No. NP_{—}060372; and human vHL amino acid sequences as set forth for example in GenPept Accession Nos. NP_{—}000542 and NP_{—}937799; (5) nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% A sequence identity with any one of the sequences referred to in (4).

Illustrative antagonist nucleic acid molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic acid molecules can interact with any macromolecule, such as DNA, RNA, polypeptides, or carbohydrate chains. Thus, antagonist nucleic acid molecules can interact with PHD, FIH-1 or vHL mRNA or the genomic DNA of PHD, FIH-1 or vHL or they can interact with the PHD, FIH-1 or vHL polypeptide. Often antagonist nucleic acid molecules are designed to interact with other nucleic acids based on sequence homology between the target molecule and the antagonist nucleic acid molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In some embodiments, anti-sense RNA or DNA molecules are used to directly block the translation of PHD, FIH-1 or vHL mRNA by binding to targeted mRNA and preventing protein translation. Antisense molecules are designed to interact with a target nucleic acid molecule through either canonical or non-canonical base pairing. The interaction of the antisense molecule and the target molecule may be designed to promote the destruction of the target molecule through, for example, RNAseH mediated RNA-DNA hybrid degradation. Alternatively the antisense molecule may be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Non-limiting methods include in vitro selection experiments and DNA modification studies using dimethylsulfate (DMS) and diethylpyrocarbonate (DEPC). In specific examples, the antisense molecules bind the target molecule with a dissociation constant (Kd) less than or equal to 10⁻⁶, 10⁻⁸, 10⁻¹⁰, or 10⁻¹². In specific embodiments, antisense oligodeoxyribonucleotides derived from the translation initiation site, e.g., between −10 and +10 regions are employed.

Aptamers are molecules that interact with a target molecule, suitably in a specific way. Aptamers are generally small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kds from the target molecule of less than 10⁻¹² M. Suitably, the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is desirable that an aptamer have a Kd with the target molecule at least 10-, 100-, 1000-, 10,000-, or 100,000-fold lower than the K_{d} with a background-binding molecule. A suitable method for generating an aptamer to a target of interest (e.g., PHD, FIH-1 or vHL) is the “Systematic Evolution of Ligands by EXponential Enrichment” (SELEX™). The SELEX™ method is described in U.S. Pat. No. 5,475,096 and U.S. Pat. No. 5,270,163 (see also WO 91/19813). Briefly, a mixture of nucleic acids is contacted with the target molecule under conditions favorable for binding. The unbound nucleic acids are partitioned from the bound nucleic acids, and the nucleic acid-target complexes are dissociated. Then the dissociated nucleic acids are amplified to yield a ligand-enriched mixture of nucleic acids, which is subjected to repeated cycles of binding, partitioning, dissociating and amplifying as desired to yield highly specific high affinity nucleic acid ligands to the target molecule.

In other embodiments, anti-PHD, -FIH-1 or -vHL ribozymes are used for catalyzing the specific cleavage of PHD, FIH-1 or vHL RNA. The mechanism of ribozyme action involves sequence specific hybridization of the ribozyme molecule to complementary target RNA, followed by a endonucleolytic cleavage. There are several different types of ribozymes that catalyze nuclease or nucleic acid polymerase type reactions, which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Representative ribozymes cleave RNA or DNA substrates. In some embodiments, ribozymes that cleave RNA substrates are employed. Specific ribozyme cleavage sites within potential RNA targets are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base pairing. Triplex molecules are preferred because they can bind target regions with high affinity and specificity. It is generally desirable that the triplex forming molecules bind the target molecule with a Kd less than 10⁻⁶, 10⁻⁸, 10⁻¹⁰, or 10⁻¹².

External guide sequences (EGSs) are molecules that bind a target nucleic acid molecule forming a complex, and this complex is recognized by RNAse P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells.

In other embodiments, RNA molecules that mediate RNA interference (RNAi) of a PHD, FIH-1 or vHL gene or PHD, FIH-1 or vHL transcript can be used to reduce or abrogate gene expression. RNAi refers to interference with or destruction of the product of a target gene by introducing a single-stranded or usually a double-stranded RNA (dsRNA) that is homologous to the transcript of a target gene. RNAi methods, including double-stranded RNA interference (dsRNAi) or small interfering RNA (siRNA), have been extensively documented in a number of organisms, including mammalian cells and the nematode C. elegans (Fire et al., 1998, Nature, 391, 806-811). In mammalian cells, RNAi can be triggered by 21- to 23-nucleotide (nt) duplexes of small interfering RNA (siRNA) (Chiu et al., 2002, Mol. Cell. 10:549-561; Elbashir et al., 2001, Nature 411:494-498), or by micro-RNAs (miRNA), functional small-hairpin RNA (shRNA), or other dsRNAs which are expressed in vivo using DNA templates with RNA polymerase 11 promoters (Zeng et al., 2002, Mol. Cell 9:1327-1333; Paddison at al., 2002, Genes Dev. 16:948-958; Lee et al., 2002, Nature Biotechnol. 20:500-505; Paul et al., Nature Biotechnol. 2002, 20:505-508; Tuschl, T., 2002, Nature Biotechnol. 20:440-448; Yu et al., 2002, Proc. Natl. Acad. Sci. USA 99(9):6047-6052; McManus et al., 2002, RNA 8:842-850; Sui et al., 2002, Proc. Natl. Acad. Sci. USA 99(6):5515-5520).

In specific embodiments, dsRNA per se and especially dsRNA-producing constructs corresponding to at least a portion of a PHD, FIH-1 or vHL gene are used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a PHD, FIH-1 or vHL gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts, which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are suitably at least about 100 nucleotides in length. RNA-encoding constructs that express a single RNA designed to form a dsRNA via internal folding are usually at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors.

In some embodiments, RNA molecules of about 21 to about 23 nucleotides, which direct cleavage of specific mRNA to which they correspond, as for example described by Tuschl et al. in U.S. 2002/0086356, can be utilized for mediating RNAi. Such 21- to 23-nt RNA molecules can comprise a 3′ hydroxyl group, can be single-stranded or double stranded (as two 21- to 23-nt RNAs) wherein the dsRNA molecules can be blunt ended or comprise overhanging ends (e.g., 5′, 3′).

In some embodiments, the antagonist nucleic acid molecule is a siRNA. siRNAs can be prepared by any suitable method. For example, reference may be made to International Publication WO 02/44321, which discloses siRNAs capable of sequence-specific degradation of target mRNAs when base-paired with 3′ overhanging ends, which is incorporated by reference herein. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling. Va.), MWB Biotech (Esbersberg, Germany), Proligo (Boulder, Colo.), and Qiagen (Vento, The Netherlands). siRNA can also be synthesized in vitro using kits such as Ambion's SILENCER™ siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

Illustrative RNAi molecules (e.g., PHD, FIH-1 or vHL siRNA and shRNA) are available commercially from Santa Cruz Biotechnology, Inc. (Santa Cruz, Calif., USA).

In some embodiments, the HIF-α potentiating agent is an inhibitor of HIF hydroxylase enzyme, particularly an inhibitor of a HIF prolyl hydroxylase enzyme. A compound that inhibits the activity of a HIF hydroxylase enzyme refers to any compound that reduces, eliminates, or attenuates the activity of at least one HIF hydroxylase enzyme (e.g., PHD1-3). In some embodiments, the HIF-α potentiating agent is an inhibitor of a HIF prolyl hydroxylase enzyme. Methods for determining whether a compound inhibits HIF hydroxylase activity are well known in the art.

Functionally, HIF hydroxylase inhibitors for use in the methods of the present invention are defined by their ability to inhibit an activity of a 2-oxoglutarate dioxygenase enzyme, wherein the enzyme has specific activity toward hypoxia inducible factor. Such compounds are often referred to as HIF hydroxylase inhibitors, HIF prolyl hydroxylase inhibitors, HIF prolyl-4-hydroxylase inhibitors, prolyl hydroxylase inhibitors or “PHI”s. In specific embodiments, the PHIs for use in the invention are small molecule compounds. A compound that inhibits the activity of a HIF hydroxylase enzyme may additionally show inhibitory activity toward one or more other 2-oxoglutarate- and iron-dependent dioxygenase enzymes, e.g., FIH (GenBank Accession No. AAL27308), procollagen prolyl 4-hydroxylase (CP4H), etc.

In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron. PHIs are typically heterocyclic carboxamide compounds, especially heterocyclic carbonyl glycine derivatives, and may be, for example, a heterocyclic carboxamide, including pyridine, pyrimidine, pyridazine, naphthyridine, pyrrolopyridine, thiazolopyridine, isothiazolopyridine, quinoline, isoquinoline, cinnoline, beta-carboline, quinolone, thienopyridine, chromene, or thiochromene carboxamides. More particularly, the inhibitor may be a heterocyclic carbonyl glycine.

Compounds that inhibit HIF prolyl hydroxylase are known in the art and are described, inter alia, in U.S. Pat. Nos. 5,658,933; 5,620,995; 5,719,164; 5,726,305; 6,093,730; 7,323,475; U.S. application Ser. No. 12/544,861; U.S. 2006/0199836; U.S. 2007/0298104; U.S. 2008/0004309; and WO 2009/073669; WO 2009/089547; WO 2009/100250; WO 02/089799; WO 02/089809; U.S. 2003/0176317, U.S. 2003/083351; U.S. 2003/0153503, U.S. 2004/0053977; U.S. Pat. No. 7,323,475, U.S. 2006/0199836, U.S. Pat. No. 8,324,208; U.S. Pat. No. 8,323,671; U.S. Pat. No. 8,343,952; U.S. Pat. No. 8,269,008; U.S. Patent Application Publication No. 2012/0309977; U.S. 2012/0329836; U.S. 2012/0316204; U.S. Patent Application Publication No. 2011/0305776; U.S. Pat. No. 7,928,120, U.S. Pat. No. 7,696,223, U.S. 2010/0303928, U.S. 2010/0330199, U.S. 2010/0331400, U.S. 2010/0047367, PCT/US2009/064065, U.S. Pat. No. 7,897,612, U.S. Pat. No. 7,608,621, U.S. Pat. No. 7,728,130, U.S. Pat. No. 7,635,715, U.S. Pat. No. 7,569,726, U.S. Pat. No. 7,811,595; U.S. 2007/0299086; U.S. 2011/0111058; U.S. 2011/0110961; U.S. Pat. No. 8,309,537; WO 2003/049686; U.S. 2003/176317; U.S. 2004/0254215; WO 2004/4108681; WO 2005/034929; WO 2005/007192; WO 2004/108121; U.S. 2005/020487; WO 2003/053997; U.S. 2003/153503; WO 2007/070359; U.S. 2009/0111806; U.S. Pat. No. 8,124,775; U.S. 2009/0093483; U.S. 2009/0156605; U.S. 2009/0088475; U.S. 2009/0099171; WO 2008/137060; U.S. 2009/0156633; U.S. 2010/0035906; WO 2008/049538; WO 2008/067871; U.S. 2010/0093803; U.S. 2009/269420; WO 2011/006355; WO 2011/106226; U.S. 2011/028507; WO 2010/018458; WO 2011/056725; WO 2011/049126; WO 2011/049127; WO 2007/038571; U.S. 2009/0082357; WO 07/136990; WO 09/039323; U.S. 2009/0176825; U.S. 2010/0113444; WO 08/089051; U.S. Ser. No. 08/017,1756; WO 08/089052; WO 2009/039321; WO 2009/039322; U.S. 2009/0176825; WO 09/049112; U.S. 2010/0305154; U.S. 2010/0305133; U.S. 2010/0298324; WO 2009/134847; U.S. 2011/0039895; U.S. 2011/0098324; U.S. 2011/0160227; WO 2010/022308; U.S. 2011/0144167; WO 2010/059549; WO 2010/059552; WO 2010/059555; U.S. 2011/0046132; WO 2009/134754; U.S. 2010/0204226; WO 2012/021830; U.S. 2011/0077267; U.S. 2012/004197; U.S. 2010/0056563; U.S. 2010/0137297; U.S. 2010/0331358; U.S. 2011/009425; U.S. 2011/009406; U.S. 2009/0239876; U.S. 2011/0152304; WO 2010/147776; WO 2011/002623; WO 2011/002624; WO 2011/133444; WO 2011/130908; WO 2010/076524; WO 2010/076525; WO 2011/045811; U.S. 2011/0130414; WO 2011/048611; WO 2012/106472; WO 2013/013609; WO 2013/017063; JP 04/083570; WO 09/131127; U.S. 2011/112103; U.S. 2012/220609; U.S. 2006/040986; U.S. 2004/0053918; U.S. 2012/0108581; U.S. Pat. No. 8,471,024; U.S. Pat. No. 5,985,913; and U.S. 2009/0048294. The foregoing patents and patent applications are incorporated herein by reference in their entireties. In one aspect, the present invention specifically contemplates the use of one or more of the compounds that are described and/or specifically exemplified or claimed in any of the foregoing patents and patent applications.

In one embodiment of the invention, the HIF-α potentiating agent is selected from the group consisting of [(4-Hydroxy-7-phenylsulfanyl-isoquinoline-3-carbonyl)-amino]-acetic acid (Compound X), {[5-(4-Chloro-phenoxy)-1-cyano-4-hydroxy-isoquinoline-3-carbonyl]-amino}-acetic acid (Compound A), [(1-Cyano-4-hydroxy-5-phenoxy-isoquinoline-3-carbonyl)-amino]-acetic acid (Compound B), {[7-Cyano-1-(2-fluoro-benzyl)-4-hydroxy-1H-pyrrolo[2,3-c]pyridine-5-carbonyl]-amino}-acetic acid (Compound C), [(1,3-Dicyclohexyl-6-hydroxy-2,4-dioxo-1,2,3,4-tetrahydro-pyrimidine-5-carbonyl)-amino]-acetic acid (Compound D), {[2-(3,4′-Difluoro-biphenyl-4-ylmethyl)-5-hydroxy-6-isopropyl-3-oxo-2,3-dihydro-pyridazine-4-carbonyl]-amino}-acetic acid (Compound E), 2-(6-Morpholin-4-yl-pyrimidin-4-yl)-4-[1,2,3]triazol-1-yl-1,2-dihydro-pyrazol-3-one (Compound F), [(4-Hydroxy-7-phenoxy-isoquinoline-3-carbonyl)-amino]-acetic acid (Compound H), {[4-Hydroxy-7-(4-methoxy-phenoxy)-isoquinoline-3-carbonyl]-amino}-acetic acid (Compound J), and {[5-(3-Fluoro-phenyl)-3-hydroxy-pyridine-2-carbonyl]-amino}-acetic acid (Compound K). In another embodiment of the invention the HIF-α potentiating agent is selected from the group consisting of [(4-Hydroxy-7-phenylsulfanyl-isoquinoline-3-carbonyl)-amino]-acetic acid (Compound X), {[5-(4-Chloro-phenoxy)-1-cyano-4-hydroxy-isoquinoline-3-carbonyl]-amino}-acetic acid (Compound A), [(1-Cyano-4-hydroxy-5-phenoxy-isoquinoline-3-carbonyl)-amino]-acetic acid (Compound B), {[7-Cyano-1-(2-fluoro-benzyl)-4-hydroxy-1H-pyrrolo[2,3-c]pyridine-5-carbonyl]-amino}-acetic acid (Compound C), [(4-Hydroxy-7-phenoxy-isoquinoline-3-carbonyl)-amino]-acetic acid (Compound H), and {[4-Hydroxy-7-(4-methoxy-phenoxy)-isoquinoline-3-carbonyl]-amino}-acetic acid (Compound J).

Other prolyl hydroxylase inhibitors are well known and have been described, inter alia, Bioorg Med Chem Lett. 16(21):5616-20 (2006); Bioorg Med Chem Lett. 16(21):5517-22 (2006); Bioorg Med Chem Lett. 16(21):5598-601 (2006); Bioorg Med Chem Lett. 16(21):5687-90 (2006); Analytical Biochemistry (2008) 384(2):213-23, 2009; J. Comb. Chem (2010) 12(5):676-86; J Cardiovase Pharmacol. 2010 August; 56(2):147-55

Methods of determining if any particular compound inhibits HIP prolyl hydroxylase are well known, illustrative examples of which include the methods described in U.S. Pat. No. 7,323,475. The inhibitory activity of any particular compound can be conveniently evaluated and compared by determining the IC₅₀ for one or more of the HIF prolyl hydroxylase enzymes. The IC₅₀ for any compound for each of the HIF prolyl hydroxylase enzymes can be determined using assays known in the art. In general, the IC₅₀ values for compounds that inhibit HIF prolyl hydroxylase will be in the μM range or less, typically in the nM range, for one or more of the HIF prolyl hydroxylase enzymes. The IC₅₀ for inhibition of the PHD2 enzyme of Compounds A, B, C, D, E, F, H, J, K, and X range from 0.05-1.5 μM. The IC₅₀ of the exemplified compounds for the PHD1 and PHD3 enzymes are in similar ranges.

Illustrative small molecule PHs include, for example, the nitrogen-containing heteroaryl compounds disclosed in U.S. 2004/0254215 (WO 2004/4108681) and in U.S. Pat. Nos. 7,323,475; 7,629,357; 7,863,292; and 8,017,625, each of which is expressly incorporated herein by reference in its entirety. Exemplary compounds of this type are represented by formula I:

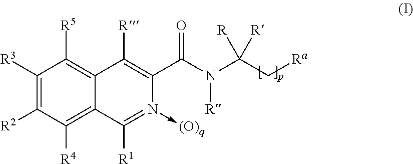

wherein:

q is zero or one;

p is zero or one;

R^{a} is —COOH or —WR⁸; provided that when R^{a} is —COOH then p is zero and when R^{a} is —WR^{a} then p is one;

W is selected from the group consisting of oxygen, —S(O)ₙ— and —NR⁹— where n is zero, one or two,

R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R is not hydrogen;

R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R² and R³ are independently selected from the group consisting of hydrogen, alkyl, substituted alkyl, aryl, substituted aryl, heteroaryl, substituted heteroaryl, halo, hydroxy, cyano, —S(O)ₙ—(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R⁶ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;

R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R is selected from the group consisting of hydrogen, deuterium and methyl;

R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;

R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O), —R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;

and pharmaceutically acceptable salts, esters and prodrugs thereof.

In an alternative embodiment, the compounds of formula I are represented by formula IA:

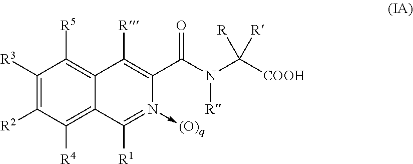

wherein R¹, R², R³, R⁴, R⁵, R, R′, R″, R′″ and q are as defined above; and

pharmaceutically acceptable salts, esters, prodrugs thereof.

In an another alternative embodiment, the compounds of formula I are represented by the formula IB:

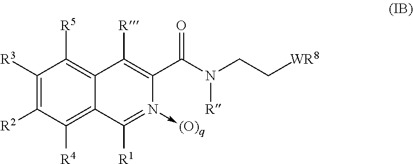

wherein R¹, R², R³, R⁴, R⁵, R″, R′″, WR⁸ and q are as defined above; and

pharmaceutically acceptable salts, esters, prodrugs thereof.

In an another alternative embodiment, the invention is directed to compounds represented by the formula IC:

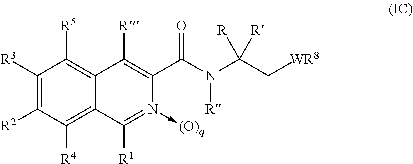

wherein R¹, R², R³, R⁴, R⁵, R, R′, R″, R′″, WR⁸ and q are as defined above; and pharmaceutically acceptable salts, esters, prodrugs thereof.

In yet another alternative embodiment, the invention is directed to compounds represented by the formula ID:

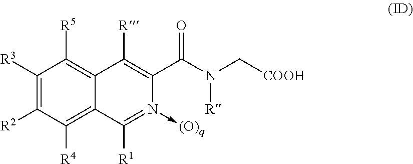

wherein R¹, R², R³, R⁴, R⁵, R, R′, R″, R′″ and q are as defined above;

and pharmaceutically acceptable salts, esters, prodrugs thereof.

Exemplary compounds according to the above formulae include {[4-Hydroxy-1-(naphthalen-2-yloxy)-isoquinoline-3-carbonyl]-amino}-acetic acid; {[4-Hydroxy-1-(pyridin-3-yloxy)-isoquinoline-3-carbonyl]-amino}-acetic acid; {[4-Hydroxy-1-(4-methoxy-phenoxy) isoquinoline-3-carbonyl]-amino-}-acetic acid; {[4-Hydroxy-1-(3-methoxy-phenoxy)-isoquinoline-3-carbonyl]-amino}-acetic acid; {[1-(3-Fluoro-phenoxy)-4-hydroxy-isoquinoline-3-carbonyl]-amino}-acetic acid; {[1-(4-Fluoro-phenoxy)-4-hydroxy-isoquinoline-3-carbonyl]-amino-}-acetic acid; {[1-(2-Fluoro-phenoxy)-4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-ace-tic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy-4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy-4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-1-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino-]-acetic acid; [(4-Ethoxy-1-methyl-7-phenoxy-isoquinoline-3-carbonyl)-amino]-acetic acid; [(1-Dimethylcarbamoyl-4-hydroxy-7-phenoxy-isoquinoline-3-carbonyl)amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid (Compound J); {[(1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinolin-3-carbonyl]-amino}-acetic acid; {[4-Hydroxy-7-(4-trifluoromethyl-phenoxy)-isoquinoline-3-carbonyl]-amino}-acetic acid; {[1-Chloro-4-hydroxy-6-(4 trifluoromethyl-phenoxy)-isoquinoline-3-carbonyl-1]-amino}-acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino}acetic acid; {[7-(4-Fluoro-phenoxy)-4-hydroxy-isoquinoline-3-carbonyl]-amino}-acetic acid; {[1-Chloro-6-(4-fluor-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin-4-ylsulfanyl)-isoquinoline-3-carbonyl]-amino}-acetic acid; {[4-Hydroxy-6-(pyridin-4-ylsulfanyl)-isoquinoline-3-carbonyl]-amino}-acetic acid; [(7-Benzenesulfinyl-4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzenesulfinyl-4-hydroxy-isoquinoline-3-carbonyl)-amino]-acetic acid; [(6-Benzenesulfonyl-4-hydroxy-isoquinoline-3-carbonyl)-amino]-acetic acid; [(6-Amino-4-hydroxy-isoquinoline-3-carbonyl)-amino]-acetic acid; {[4-Hydroxy-7-(4-methoxy-benzenesulfonylamino}-isoquinoline-3-carbonyl)-amino]-acetic acid; {[4-Hydroxy-7-(3-phenyl-ureido)-isoquinoline-3-carbonyl]-amino}-acetic acid; {[4-Hydroxy-6-(3-phenyl-ureido)-isoquinoline-3-carbonyl]-amino}-acetic acid; [(4-Hydroxy-1-phenylsulfanyl isoquinoline-3-carbonyl)-amino]-acetic acid; {[1-(4-Chloro-phenylsulfanyl) 4-hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino-}-acetic acid; {[4-Hydroxy-1-(3-methoxy-phenylsulfanyl)-isoquinoline-3-carbonyl]-amino}-acetic acid; {[4-Hydroxy-1-(2-methoxy-phenysulfanyl)-isoquinoline-3-carbonyl]-amino}-acetic acid; {[4-Hydroxy-1-(naphthalen-2-ylsulfanyl)-isoquinoline-3-carbonyl]-amino}-acetic acid; [(1-Benzenesulfinyl-4-hydroxy-isoquinoline-3-carbonyl)-amino]-acetic acid; [(1-Benzenesulfonyl-4-hydroxy-isoquinoline-3-carbonyl)-amino]-acetic acid; {[4-Hydroxy-7-(pyridin-2-ylsulfanyl)-isoquinoline-3-carbonyl]-amino}-acetic acid; {[4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino-]-acetic acid; [(4-Hydroxy-6,7-diphenoxy-isoquinoline-3-carbonyl)-amino]-acetic acid; ({4-Hydroxy-7-[4-(toluene-4-sulfonylamino)-phenoxy]-isoquinoline-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl)-amino}-acetic acid; {(4-Hydroxy-7-(4-methanesulfonylamino-phenoxy)-isoquinoline-3-carbonyl]-amino}-acetic acid; {[7-(4-Chloro-phenoxy)-4-hydroxy-isoquinoline-3-carbonyl)-amino}-acetic acid; {[6-(4-Chloro-phenoxy)-4-hydroxy-isoquinoline-3-carbonyl]-amino}-acetic acid; {[6-(3-Fluoro-5-methoxy-phenoxy)-4-hydroxy-isoquinoline-3-carbonyl]-amino}-acetic acid; {[7-(3-Fluoro-5-methoxy-phenoxy)-4-hydroxy-isoquinoline-3-carbonyl]-amino}-acetic acid; {[7-(3,4-Difluoro-phenoxy)-4-hydroxy-isoquinoline-3-carbonyl]-amino}-acetic acid; {[6-(3,4-Difluoro-phenoxy)-4-hydroxy-isoquinoline-3-carbonyl]-amino}-acetic acid; {[4-Hydroxy-7-(4-trifluoromethoxy-phenoxy)-isoquinoline-3-carbonyl]-amino}-acetic acid; {[4-Hydroxy-6-(4-trifluoromethoxy-phenoxy)-isoquinoline-3-carbonyl]-amino}-acetic acid; 2-(S)-{[7-(4-Chloro-phenoxy)-4-hydroxy-isoquinoline-3-carbonyl]-amino}-propionic acid; 2-(S)-{[6-(4-Chloro-phenoxy)-4-hydroxy-isoquinoline-3-carbonyl]-amino}-propionic acid; 2-{[7-(3,4-Difluoro-phenoxy)-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-4-Hydroxy-{[4-methoxy-phenoxy)-isoquinoline-3-carbonyl]-amino}-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-{[1-(4-Chloro-phenylsulfanyl)-4-hydroxy-isoquinoline-3-carbonyl]-amino}-propionic acid; (R)-2-{[1-(4-Chloro-phenylsulfanyl)-4-hydroxy-isoquinoline-3-carbonyl]-amino}-propionic acid; [(4-Hydroxy-7-phenylsulfanyl-isoquinoline-3-carbonyl)-amino]-acetic acid (Compound X); [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-ace-tic acid; [(1-Chloro-4-hydroxy-6-phenylsulfanyl-isoquinoline-3-carbonyl)-amino]-acetic acid; [(1-Bromo-4-hydroxy-7-phenylsulfanyl-isoquinoline-3-carbonyl-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid (Compound H); [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-ace-tic acid; [(1-Bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-Bromo-7-(2,6-dimethyl-phenoxy)-4-hydroxy-isoquinoline-3-carbonyl]-amino}-acetic acid; [(1-Bromo-7-chloro-4-hydroxy-isoquinoline-3-carbonyl)-amino]-acetic acid; [(1-Bromo-6-chloro-4-hydroxy-isoquinoline-3-carbonyl)-amino]-acetic acid; [(1-Bromo-4-hydroxy-7-trifluoromethyl-isoquinoline-3-carbonyl)-amino]-acetic acid; [(1-Bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-Bromo-1-chloro-4-hydroxy-isoquinoline-3-carbonyl)-amino]-acetic acid; [(6-Bromo-4-hydroxy-isoquinoline-3-carbonyl)-amino]-acetic acid; [(1-Bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-Bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4-hydroxy-7-phenyl-isoquinoline-3-carbonyl)-amino]-acetic acid; [(1-Bromo-4-hydroxy-6-phenyl-isoquinoline-3-carbonyl)-amino]-acetic acid; [(1-Bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-Bromo-4-hydroxy-5-phenyl-isoquinoline-3-carbonyl)-amino]-acetic acid; [(1-Bromo-4-hydroxy-8-phenyl-isoquinoline-3-carbonyl)-amino]-aceti-c acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(4-Bromo-4-hydroxy-7-methyl-isoquinoline-3-carbonyl)-amino]-acetic acid; [(1-Bromo-7-butoxy-4-hydroxy-isoquinoline-3-carbonyl)-amino]-acetic acid; [(1-Bromo-6-butoxy-4-hydroxy-isoquinoline-3-carbonyl)-amino]-acetic acid; [(6-Benzyloxy-6-chloro-4-hydroxy-isoquinoline-3-carbonyl)-methyl-amino]-acetic acid; [(1-chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3-carbonyl)-methyl-amino]-acetic acid; [Carboxymethyl-(1-chloro-4-hydroxy-isoquinoline-3-carbonyl)-amino]-acetic acid; [Carboxymethyl-(1-chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide(trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-4-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoro-acetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinoline-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1 Chloro-4-hydroxy-7-isopropoxy-isoquinoline-3-carbonyl)-amino]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid(trifluoro-acetic acid salt); (S)-2-[(1-Chloro-4-hydroxy-isoquinoline-3-carbonyl)-amino]-3-(1H-imidazol-4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-2-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[(1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino]-3-(4-hydroxy-phenyl)-propionic acid; (S)-2-[(1-Chloro-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopopoy-isoquinoline-3-carbonyl)-pyrrolidine-2-carboxylic acid; (R)-6-Amino-2-[(1-chloro-4-hydroxy-isoquinoline-3-carbonyl)-amino]-hexanoic acid (trifluoro-acetic acid salt); (S)-6-Amino-2-[(1-chloro-4-hydroxy-isoquinoline-3-carbonyl)-amino]-hexanoic acid (trifluoro-acetic acid salt); (R)-6-Amino-2-[(1-chloro-4-hydroxy-6-isopropoxy-isoquinoline-3-carbonyl)-amino]-hexanoic acid; trifluoroacetic acid salt; (S)-6-Amino-2-[(1-chloro-4-hydroxy-6-isopropoxy-isoquinoline-3-carbonyl)-amino]-hexanoic acid (trifluoro-acetic acid salt); (R)-6-Amino-2-[(1-chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline-3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; 1-[(1-Chloro-4-hydroxy-6-isopropoxy-isoquinoline-3-carbonyl)-amino]-cyclopropanecarboxylic acid; Dideutero-[(1-chloro-4-hydroxy-isoquinoline-3-carbonyl)-amino]-acetic acid; (R)-2-[(6-Benzyloxy-1-chloro-4-hydroxy-isoquinoline-3-carbonyl)-amino]-propionic acid; (S)-2-[(7-Benzyloxy-1-chloro-4-hydroxy-isoquinoline-3-carbonyl)-amino]-propionic acid; (R)-2-[(7-Benzyloxy-1-chloro-4-hydroxy-isoquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-chloro-4-hydroxy-isoquinoline-3-carbonyl)-amino]-propionic acid; (R)-2-[6-Isopropoxy-1-chloro-4-hydroxy-isoquinoline-3-carbonyl)-amino]-propionic acid; (S)-2-[(7-Isopropoxy-1-chloro-4-hydroxy-isoquinoline-3-carbonyl)-amino]-propionic acid; (R)-2-[(7-Isopropoxy-1-chloro-4-hydroxy-isoquinoline-3-carbonyl)-amino]-propionic acid; 1-Chloro-4-hydroxy-6-isopropoxy-isoquinoline-3-carboxylic acid (2-hydroxy-1-hydroxymethyl-ethyl)-amide; 1-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {[6-(3,5-Difluoro-phenoxy)-4-hydroxy-isoquinoline-3-carbonyl]-amino}-acetic acid; ({7-[4-(4-Fluoro-phenoxy)-phenoxy]-4-hydroxy-isoquinoline-3-carbonyl}-amino)-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)-4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-Fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-{[7-(4-Fluoro-phenoxy)-4-hydroxy-1-methyl-isoquinoline-3-carbonyl]-amino}-propionic acid; 2-(S)-{[7-(4-Fluoro-phenoxy)-4-hydroxy-isoquinoline-3-carbonyl]-amino}-propionic acid; 2-(S)-[(4-Hydroxy-1-methyl-7-phenoxy-isoquinoline-3-carbonyl)-amino]-propionic acid; 2-(S)-[(4-Hydroxy-1-methyl-7-phenylsulfanyl-isoquinoline-3-carbonyl)-amino]-propionic acid; 2-(S)-{[4-Hydroxy-7-(4-trifluoromethyl-phenoxy)-isoquinoline-3-carbonyl]-amino}-propionic acid; {[7-(4-Chloro-phenoxy)-4-hydroxy-1-methyl-isoquinoline-3-carbonyl]-amino}-acetic acid; {[6-(4-Chloro-phenoxy)-4-hydroxy-1-methyl-isoquinoline-3-carbonyl]-amino}-acetic acid; {[7-(3,5-Difluoro-phenoxy)-4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino-}-acetic acid; {[4-Hydroxy-6-(4-methoxy-phenoxy)-1-methyl-isoquinoline-3-carbonyl]-amino-}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyl-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-ace-tic acid; {[4-Hydroxy-1-methyl-7-(4-trifluoromethyl-phenoxy)-isoquinoline-3-carbonyl]-amino}-acetic acid; and pharmaceutically acceptable salts, esters and prodrugs thereof.

In some embodiments, small molecule PHIs may be selected from cyanoisoquinoline compounds disclosed in U.S. Pat. No. 7,928,120, which is expressly incorporated herein by reference in its entirety. These compounds can be represented by formula II:

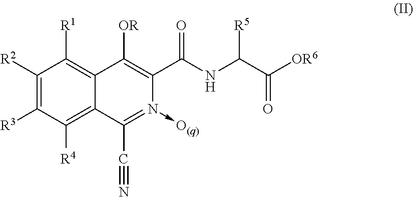

wherein:

R is selected from the group consisting of hydrogen, alkyl, and substituted alkyl;

R¹, R², R³ and R⁴ are independently selected from the group consisting of hydrogen, halo, cyano, hydroxyl, alkyl, substituted alkyl, aryl, substituted aryl, heteroaryl, substituted heteroaryl, amino, substituted amino, —OR⁷, —SR⁷, —SOR⁷, and —SO₂R⁷ wherein R⁷ is selected from the group consisting of alkyl, substituted alkyl, cycloalkyl, substituted cycloalkyl, aryl, substituted aryl, heteroaryl, and substituted heteroaryl; and

R⁵ and R⁶ are independently selected from the group consisting of hydrogen or C₁₋₃ alkyl;

or pharmaceutically acceptable salts, tautomers, stereoisomers, solvates, and/or prodrugs thereof.

Representative compounds of Formula II include;

{[1-cyano-4-hydroxy-isoquinoline-3-carbonyl]-amino}-acetic acid, 2-(S)-[(1-cyano-4-hydroxy-isoquinoline-3-carbonyl)-amino]-propionic acid, {[1-cyano-4-hydroxy-7-phenoxy-isoquinoline-3-carbonyl]-amino}-acetic acid, 2-(S)-[(1-cyano-4-hydroxy-7-phenoxy-isoquinoline-3-carbonyl)-amino]-propionic acid, 2-(R)-[(1-cyano-4-hydroxy-7-phenoxy-isoquinoline-3-carbonyl)-amino]-propionic acid, {[1-cyano-7-(4-fluorophenoxy)-4-hydroxy-isoquinoline-3-carbonyl]-amino}-acetic acid, {[1-cyano-4-hydroxy-7-(trifluoromethyl)-isoquinoline-3-carbonyl]-amino}-acetic acid, {[1-cyano-4-hydroxy-7-chloro-isoquinoline-3-carbonyl]-amino}-acetic acid, {[i-cyano-4-hydroxy-8-phenoxy-isoquinoline-3-carbonyl]-amino}-acetic acid, {[1-cyano-8-(4-fluoro-phenoxy)-4-hydroxy-isoquinoline-3-carbonyl]-amino}-acetic acid, [(1-cyano-4-hydroxy-6-methoxy-isoquinoline-3-carbonyl)-amino]-acetic acid, [(1-cyano-4-hydroxy-6-phenoxy-isoquinoline-3-carbonyl)-amino]-acetic acid, {[1-cyano-6-(4-fluoro-phenoxy)-4-hydroxy-isoquinoline-3-carbonyl]-amino}-acetic acid, {[1-cyano-4-hydroxy-6-(4-methoxy-phenoxy)-isoquinoline-3-carbonyl]-amino}-acetic acid, [(1-cyano-4-hydroxy-6-phenylsulfanyl-isoquinoline-3-carbonyl)-amino]-acetic acid, [(1-cyano-4-hydroxy-7-phenylsulfanyl-isoquinoline-3-carbonyl)-amino]-acetic acid, {[1-cyano-6-(2,6-dimethyl-phenoxy)-4-hydroxy-isoquinoline-3-carbonyl]-amino}-acetic acid, [(1-cyano-4-hydroxy-5-phenoxy-isoquinoline-3-carbonyl)-amino]-acetic acid (Compound B), {[1-cyano-4-hydroxy-8-(4-methoxy-phenoxy)-isoquinoline-3-carbonyl]-amino}-acetic acid, {[1-cyan-4-hydroxy-8-(3-methoxy-phenoxy)-isoquinoline-3-carbonyl]-amino}-acetic acid, {[1-cyano-4-hydroxy-8-(2-methoxy-phenoxy)-isoquinoline-3-carbonyl]-amino}-acetic acid, [(7-benzyl-1-cyano-4-hydroxy-isoquinoline-3-carbonyl)-amino]-acetic acid, {[1-cyano-5-(4-fluoro-phenoxy)-4-hydroxy-isoquinoline-3-carbonyl]-amino}-acetic acid, {[1-cyano-7-(2,6-dimethyl-phenoxy)-4-hydroxy-isoquinoline-3-carbonyl]-amino}-acetic acid, {[1-cyano-6-(2-ethyl-6-methyl-phenoxy)-4-hydroxy-isoquinoline-3-carbonyl]-amino}-acetic acid, {[1-cyano-4-hydroxy-6-(2,4,6-trimethyl-phenoxy)-isoquinoline-3-carbonyl]-amino}-acetic acid, {[6-(4-chloro-2,6-dimethyl-phenoxy)-1-cyano-4-hydroxy-isoquinoline-3-carbonyl]-amino}-acetic acid, {[1-cyano-4-hydroxy-7-(4-methoxy-phenoxy)-isoquinoline-3-carbonyl]-amino}-acetic acid, [(1-cyan-6-cyclohexyloxy-4-hydroxy-isoquinoline-3-carbonyl)-amino]-acetic acid, [(6-benzenesulfonyl-1-cyano-4-hydroxy-isoquinoline-3-carbonyl)-amino]-acetic acid, {[1-cyano-4-hydroxy-6-(4-propoxy-phenoxy)-isoquinoline-3-carbonyl]-amino}-acetic acid, {[7-(benzo[1,3]dioxol-5-yloxy)-1-cyano-4-hydroxy-isoquinoline-3-carbonyl]-amino}-acetic acid, {[6-(benzo[1,3]dioxol-5-yloxy)-1-cyano-4-hydroxy-isoquinoline-3-carbonyl]-amino}-acetic acid, {[1-cyano-6-(2,3-dihydro-benzofuran-5-yloxy)-4-hydroxy-isoquinoline-3-carbonyl]-amino}-acetic acid, [(1-cyano-4-methoxy-no-8-phenoxy-isoquinoline-3-carbonyl)-amino]-acetic acid methyl ester, [(1-cyano-4-methoxy-8-phenoxy-isoquinoline-3-carbonyl)-amino]-acetic acid, (S)-2-[(1-cyano-4-hydroxy-8-phenoxy-isoquinoline-3-carbonyl)-amino]-propionic acid, (R)-2-[(1-cyano-4-hydroxy-8-phenoxy-isoquinoline-3-carbonyl)-amino]-propionic acid, {[1-cyano-4-hydroxy-6-(2-methyl-benzothiazol-6-yloxy)-isoquinoline-3-carbonyl]-amino}-acetic acid, {[1-cyano-6-(2-dimethylamino-benzooxazol-5-yloxy)-4-hydroxy-isoquinoline-3-carbonyl]-amino}-acetic acid, {[1-cyano-7-(2-dimethylamino-benzooxazol-5-yloxy)-4-hydroxy-isoquinoline-3-carbonyl]-amino}-acetic acid, {[1-cyano-4-hydroxy-6-(2-morpholin-4-yl-benzothiazol-6-yloxy)-isoquinoline-3-carbonyl]-amino}-acetic acid, {[1-cyano-4-hydroxy-6-(2-methyl-benzooxazol-6-yloxy)-isoquinoline-3-carbonyl]-amino}-acetic acid, [(6-chloro-1-cyano-4-hydroxy-isoquinoline-3-carbonyl)-amino]-acetic acid, [(7-butoxy-1-cyano-4-hydroxy-isoquinoline-3-carbonyl)-amino]-acetic acid, [(1-cyano-4-hydroxy-6,7-diphenoxy-isoquinoline-3-carbonyl)-amino]-acetic acid, [(1-cyano-4-hydroxy-7-methoxy-isoquinoline-3-carbonyl)-amino]-acetic acid, [(1-cyano-4-hydroxy-7-isopropoxy-isoquinoline-3-carbonyl)-amino]-acetic acid, [(1-cyano-4-hydroxy-6-isopropoxy-isoquinoline-3-carbonyl)-amino]-acetic acid. [(1-cyano-4-hydroxy-5-phenyl-isoquinoline-3-carbonyl)-amino]-acetic acid, [(1-cyano-4-hydroxy-8-phenyl-isoquinoline-3-carbonyl)-amino]-acetic acid, [(7-benzyloxy-1-cyano-4-hydroxy-isoquinoline-3-carbonyl)-amino]-acetic acid, [{5-(4-chloro-phenoxy)-1-cyano-4-hydroxy-isoquinoline-3-carbonyl]-amino)-acetic acid (Compound A), and [(1-cyano-4,7-dihydroxy-isoquinoline-3-carbonyl)-amino]-acetic acid.

In other embodiments, small molecule PHIs may be selected from pyrrolo- and thiazolo-pyridine compounds disclosed in U.S. Pat. No. 7,696,223, which is expressly incorporated herein by reference in its entirety. These compounds can be represented by formula III:

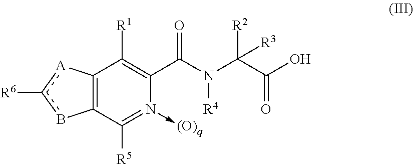

wherein:

q is 0 or 1;

A and B are independently selected from the group consisting ═C(R⁷)—, —N(R⁸)—, ═N—, and —S— with the proviso that one of the following is present:

A is ═C(R⁷)— and B is —N(R⁸)—;

A is —S— and B is ═N—;

A ═N— and B is —S—; or

A is —N(R⁸)— and B is ═C(R⁷)—;

one of -A [symbol] C(R⁶)— or —B [symbol] C(R⁶)— is a double bond and the other is a single bond;

R¹ is selected from the group consisting of hydroxyl, alkoxy, substituted alkoxy, acyloxy, cycloalkoxy, substituted cycloalkoxy, aryloxy, substituted aryloxy, heteroaryloxy, substituted heteroaryloxy, heterocyclyloxy, substituted heterocyclyloxy, mercapto, thioether, substituted alkylthio, arylsulfanyl, heteroarylsulfanyl, amino, substituted amino, acylamino and anminoacyl;

R² is selected from the group consisting of hydrogen, deuterium, and methyl;

R³ is selected from the group consisting of hydrogen, deuterium, alkyl, and substituted alkyl;

R⁴ is selected from the group consisting of hydrogen, alkyl, and substituted alkyl;

R⁵ is selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;

R⁶ and R⁷ are each independently selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;

or where when A or B is ═C(R⁷)—, then R⁶ and R⁷ together with the carbon atoms bound thereto join to form a cycloalkenyl, substituted cycloalkenyl, aryl, substituted aryl, heteroaryl, or substituted heteroaryl; and

R⁸ is selected from the group consisting of hydrogen, hydroxyl, alkyl, substituted alkyl, alkenyl, substituted alkenyl, alkynyl, substituted alkynyl, aryl, substituted aryl, heteroaryl, and substituted heteroaryl;

or pharmaceutically acceptable salts, single stereoisomers, mixtures of stereoisomers, esters, or prodrugs thereof.

Representative compounds of Formula III include:

[(2-bromo-4-hydroxy-1-phenyl-1H-pyrrolo[2,3-c]pyridine-5-carbonyl)-amino]-acetic acid, [(4-hydroxy-1-phenyl-1H-pyrrolo[2,3-c]pyridine-5-carbonyl)-amino]-acetic acid, [(2,3-dibromo-4-hydroxy-1-phenyl-1H-pyrrolo[2,3-c]pyridine-5-carbonyl)-amino]-acetic acid, {[3-bromo-2-(4-fluoro-phenyl)-4-hydroxy-1-phenyl-1H-pyrrolo[2,3-c]pyridine-5-carbonyl]-amino}-acetic acid, {[(1-benzyl-2,3-dibromo-4-hydroxy-1H-pyrrolo[2,3-c]pyridine-5-carbonyl]-amino}-acetic acid, {[2-(4-fluoro-phenyl)-4-hydroxy-1-phenyl-1H-pyrrolo[2,3-c]pyridine-5-carbonyl]-amino}-acetic acid, [(1-benzyl-4-hydroxy-1H-pyrrolo[2,3-c]pyridine-5-carbonyl)-amino]-acetic acid, {[3-bromo-1,2-bis-(4-fluoro-phenyl)-4-hydroxy-1H-pyrrolo[2,3-c]pyridine-5-carbonyl]-amino}-acetic acid, {[1,2-bis-(4-fluoro-phenyl)-4-hydroxy-1H-pyrrolo[2,3-c]pyridine-5-carbonyl]-amino}-acetic acid, {[3-chloro-1,2-bis-(4-fluoro-phenyl)-4-hydroxy-1H-pyrrolo[2,3-c]pyridine-5-carbonyl]-amino}-acetic acid, {[3-bromo-2-(4-fluoro-phenyl)-4-hydroxy-1-(4-methoxy-phenyl)-1H-pyrrolo[2,3-c]pyridine-5-carbonyl]-amino}-acetic acid, {[2-(4-fluoro-phenyl)-4-hydroxy-1-(4-methoxy-phenyl)-1H-pyrrolo[2,3-c]pyridine-5-carbonyl]-amino}-acetic acid, {[2-bromo-1-(4-fluoro-phenyl)-4-hydroxy-3-phenyl-1H-pyrrolo[2,3-c]pyridine-5-carbonyl]-amino}-acetic acid, {[1-(4-fluoro-phenyl)-4-hydroxy-3-phenyl-1H-pyrrolo[2,3-c]pyridine-5-carbonyl]-amino}-acetic acid, {[7-chloro-1-(4-fluoro-phenyl)-4-hydroxy-3-phenyl-1H-pyrrolo[2,3-c]pyridine-5-carbonyl]-amino}-acetic acid, {[7-methyl-1-(4-fluoro-phenyl)-4-hydroxy-3-phenyl-1H-pyrrolo[2,3-c]pyridine-5-carbonyl]-amino}-acetic acid, {[3-bromo-2-tert-butyl-1-(4-fluoro-phenyl)-4-hydroxy-1H-pyrrolo[2,3-c]pyridine-5-carbonyl]-amino}-acetic acid, {[2-tert-butyl-1-(4-fluoro-phenyl)-4-hydroxy-1H-pyrrolo[2,3-c]pyridine-5-carbonyl]-amino}-acetic acid, [(1-benzyl-4-hydroxy-2,3-dimethyl-1H-pyrrolo[2,3-c]pyridine-5-carbonyl)-amino]-acetic acid, [(2,3-dibromo-4-hydroxy-1-methyl-1H-pyrrolo[2,3-c]pyridine-5-carbonyl)-amino]-acetic acid, [(4-hydroxy-1,2,3-trimethyl-1H-pyrrolo[2,3-c]pyridine-5-carbonyl)-amino]-acetic acid, {[2-bromo-3-tert-buty-1-(4-fluoro-phenyl)-4-hydroxy-1H-pyrrolo[2,3-c]pyridine-5-carbonyl]-amino}-acetic acid, {[3-tert-butyl-1-(4-fluoro-phenyl)-4-hydroxy-1H-pyrrolo[2,3-c]pyridine-5-carbonyl]-amino}-acetic acid, [(1-benzyl-4-hydroxy-2,3-dipropyl-1H-pyrrolo[2,3-c]pyridine-5-carbonyl)-amino]-acetic acid, [(1-benzyl-3,7-dichloro-4-hydroxy-1H-pyrrolo[2,3-c]pyridine-5-carbonyl)-amino]-acetic acid, [(4-hydroxy-9-phenyl-9h-beta-carboline-3-carbonyl)-amino]-acetic acid, [(4-hydroxy-1-methyl-9-phenyl-9h-beta-carboline-3-carbonyl)-amino]-acetic acid, [(4-hydroxy-1,9-diphenyl-9h-beta-carboline-3-carbonyl)-amino]-acetic acid, [(1-benzyl-3-chloro-4-hydroxy-7-methyl-1H-pyrrolo[2,3-c]pyridine-5-carbonyl)-amino]-acetic acid, [(1-benzyl-3-chloro-4-hydroxy-7-phenyl-1H-pyrrolo[2,3-c]pyridine-5-carbonyl)-amino]-acetic acid, [(1-benzyl-3-chloro-7-ethyl-4-hydroxy-1H-pyrrolo[2,3-c]pyridine-5-carbonyl)-amino]-acetic acid, {[2-(4-fluoro-phenyl)-4-hydroxy-1,3-diphenyl-1H-pyrrolo[2,3-c]pyridine-5-carbonyl]-amino}-acetic acid, [(3-chloro-4-hydroxy-1-phenyl-1H-pyrrolo[2,3-c]pyridine-5-carbonyl)-amino]-acetic acid, [(3-chloro-4-hydroxy-7-methyl-1-phenyl-1H-pyrrolo[2,3-c]pyridine-5-carbonyl)-amino]-acetic acid, {[1-(benzo[1,3]dioxol-5-ylmethyl)-3-bromo-2-(4-chloro-phenyl)-4-hydroxy-1H-pyrrolo[2,3-c]pyridine-5-carbonyl]-amino}-acetic acid, {[3-bromo-2-(4-chloro-phenyl)-4-hydroxy-1-phenyl-1H-pyrrolo[2,3-c]pyridine-5-carbonyl]-amino}-acetic acid, [(1-(benzo[1,3]dioxol-5-ylmethyl)-4-hydroxy-2-phenyl-1H-pyrrolo[2,3-c]pyridine-5-carbonyl-amino]-acetic acid, {[1-(benzo[1,3]dioxol-5-ylmethyl)-2-(4-chloro-phenyl)-4-hydroxy-1H-pyrrolo[2,3-c]pyridine-5-carbonyl]-amino}-acetic acid, {[1-benzo[1,3]dioxol-5-ylmethyl-2-(4-chloro-phenyl)-4-hydroxy-3-methyl-1H-pyrrolo[2,3-c]pyridine-5-carbonyl]-amino}-acetic acid, [(4-hydroxy-1,2-diphenyl-1H-pyrrolo[2,3-c]pyridine-5-carbonyl)-amino]-acetic acid, {[2-(4-chloro-phenyl)-4-hydroxy-3-methyl-1-phenyl-1H-pyrrolo[2,3-c]pyridine-5-carbonyl]-amino}-acetic acid, [(7-hydroxy-2-phenyl-thiazolo[4,5-c]pyridine-6-carbonyl)-amino]-acetic acid, [(7-hydroxy-2,4-diphenyl-thiazolo[4,5-c]pyridine-6-carbonyl)-amino]-acetic acid, [(7-hydroxy-4-methyl-2-phenyl-thiazolo[4,5-c]pyridine-6-carbonyl)-amino]-acetic acid, (S)-2-[(7-hydroxy-4-methyl-2-phenyl-thiazolo[4,5-c]pyridine-6-carbonyl)-amino]-propionic acid, {[7-hydroxy-2-(4-trifluoromethyl-phenyl)-thiazolo[4,5-c]pyridine-6-carbonyl]-amino}-acetic acid, {[2-(4-chloro-phenyl)-7-hydroxy-thiazolo[4,5-c]pyridine-6-carbonyl]-amino}-acetic acid, {[7-hydroxy-2-(4-methoxy-phenyl)-thiazolo[4,5-c]pyridine-6-carbonyl]-amino}-acetic acid, {[2-(4-fluoro-phenyl)-7-hydroxy-thiazolo[4,5-c]pyridine-6-carbonyl]-amino}-acetic acid, [(4-ethyl-7-hydroxy-2-phenyl-thiazolo[4,5-c]pyridine-6-carbonyl)-amino]-acetic acid, [(7-hydroxy-2-phenoxy-thiazolo[4,5-c]pyridine-6-carbonyl)-amino]-acetic acid, {[7-hydroxy-2-(methyl-phenyl-amino)-thiazolo[4,5-c]pyridine-6-carbonyl]-amino}-acetic acid, {[7-hydroxy-2-(phenylamino)-thiazolo[4,5-c]pyridine-6-carbonyl]-amino}-acetic acid, [(7-hydroxy-2-phenyl-thiazolo[5,4-c]pyridine-6-carbonyl)-amino]-acetic acid, {[2-(5-bromo-pyridin-3-yl)-7-hydroxy-thiazolo[4,5-c]pyridine-6-carbonyl]-amino}-acetic acid, [(7-hydroxy-2-pyridin-3-yl-thiazolo[4,5-c]pyridine-6-carbonyl)-amino]-acetic acid, [(4-butyl-7-hydroxy-2-phenyl-thiazolo[4,5-c]pyridine-6-carbonyl)-amino]-acetic acid, [(7-hydroxy-2-pyridin-2-yl-thiazolo[4,5-c]pyridine-6-carbonyl)-amino]-acetic acid, {[2-(4-fluoro-phenyl)-7-hydroxy-4-methyl-thiazolo[4,5-c]pyridine-6-carbonyl]-amino}-acetic acid, [(7-hydroxy-2-phenyl-4-propyl-thiazolo[4,5-c]pyridine-6-carbonyl)-amino]-acetic acid, {[7-hydroxy-2-(4-phenoxy-phenyl)-thiazolo[4,5-c]pyridine-6-carbonyl]-amino}-acetic acid, [(4-cyano-7-hydroxy-2-phenyl-thiazolo[4,5-c]pyridine-6-carbonyl)-amino]-acetic acid, [(7-hydroxy-4-isobutyl-2-phenyl-thiazolo[4,5-c]pyridine-6-carbonyl)-amino]-acetic acid, {[7-hydroxy-2-(3-methoxy-phenyl)-thiazolo[4,5-c]pyridine-6-carbonyl]-amino}-acetic acid, [(4-furan-2-yl-7-hydroxy-2-phenyl-thiazolo[4,5-c]pyridine-6-carbonyl)-amino]-acetic acid, [(7-hydroxy-2-phenyl-4-thiazol-2-yl-thiazolo[4,5-c]pyridine-6-carbonyl)-amino]-acetic acid, {[7-hydroxy-2-(2-methoxy-phenyl)-thiazolo[4,5-c]pyridine-6-carbonyl]-amino}-acetic acid, [(7-hydroxy-4-methyl-2-phenyl-thiazolo[5,4-c]pyridine-6-carbonyl)-amino]-acetic acid, {[2-(4-cyano-phenyl)-7-hydroxy-thiazolo[4,5-c]pyridine-6-carbonyl]-amino}-acetic acid, [(7-hydroxy-2,4-diphenyl-thiazolo[5,4-c]pyridine-6-carbonyl)-amino]-acetic acid, {[2-(3-chloro-4-fluoro-phenyl)-7-hydroxy-thiazolo[4,5-c]pyridine-6-carbonyl]-amino}-acetic acid, [(4-benzyl-7-hydroxy-2-phenyl-thiazolo[5,4-c]pyridine-6-carbonyl)-amino]-acetic acid, {[7-hydroxy-4-(4-morpholin-4-yl-phenyl)-2-phenyl-thiazolo[5,4-c]pyridine-6-carbonyl]-amino}-acetic acid, {[4-(4-cyano-phenyl)-7-hydroxy-2-phenyl-thiazolo[4,5-c]pyridine-6-carbonyl]-amino}-acetic acid, {[4-cyano-2-(4-fluoro-phenyl)-7-hydroxy-thiazolo[4,5-c]pyridine-6-carbonyl]-amino}-acetic acid, {[4-cyano-7-hydroxy-2-(3-methoxy-phenyl)-thiazolo[4,5-c]pyridine-6-carbonyl]-amino}-acetic acid, [(4-cyano-7-hydroxy-2-phenyl-thiazolo[5,4-c]pyridine-6-carbonyl)-amino]-acetic acid, [(4-ethynyl-7-hydroxy-2-phenyl-thiazolo[4,5-c]pyridine-6-carbonyl)-amino]-acetic acid, [(4-acetyl-7-hydroxy-2-phenyl-thiazolo[4,5-c]pyridine-6-carbonyl)-amino]-acetic acid, [(7-hydroxy-2-phenyl-4-piperidin-1-yl-thiazolo[4,5-c]pyridine-6-carbonyl)-amino]-acetic acid, {[2-(4-tert-butyl-phenyl)-7-hydroxy-thiazolo[4,5-c]pyridine-6-carbonyl]-amino}-acetic acid, {[2-(2,3-dihydro-benzo[1,4]dioxin-6-yl)-7-hydroxy-thiazolo[4,5-c]pyridine-6-carbonyl]-amino}-acetic acid, [(2-benzo[b]thiophen-3-yl-7-hydroxy-thiazolo[4,5-c]pyridine-6-carbonyl)-amino]-acetic acid, [(2-biphenyl-4-yl-7-hydroxy-thiazolo[4,5-c]pyridine-6-carbonyl)-amino]-acetic acid, [(2-benzo[b]thiophen-2-yl-7-hydroxy-thiazolo[4,5-c]pyridine-6-carbonyl)-amino]-acetic acid, [(7-hydroxy-2-quinolin-3-yl-thiazolo[4,5-c]pyridine-6-carbonyl)-amino]-acetic acid, [(2-benzofuran-2-yl-7-hydroxy-thiazolo[4,5-c]pyridine-6-carbonyl)-amino]-acetic acid, [(2-dibenzofuran-4-yl-7-hydroxy-thiazolo[4,5-c]pyridine-6-carbonyl)-amino]-acetic acid, {[2-(2,3-dihydro-benzofuran-5-yl)-7-hydroxy-thiazolo[4,5-c]pyridine-6-carbonyl]-amino}-acetic acid, [(7-hydroxy-2-pyrimidin-5-yl-thiazolo[4,5-c]pyridine-6-carbonyl)-amino]-acetic acid, {[2-(1-benzyl-1H-pyrazol-4-yl)-7-hydroxy-thiazolo[4,5-c]pyridine-6-carbonyl]-amino}-acetic acid, {[2-(6-chloro-pyridin-3-yl)-7-hydroxy-thiazolo[4,5-c]pyridine-6-carbonyl]-amino}-acetic acid, {[2-(6-butoxy-pyridin-3-yl)-7-hydroxy-thiazolo[4,5-c]pyridine-6-carbonyl]-amino}-acetic acid, {[7-hydroxy-2-(6-phenylsulfanyl-pyridin-3-yl)-thiazolo[4,5-c]pyridine-6-carbonyl]-amino}-acetic acid, {[2-(1-benzyl-1H-pyrazol-4-yl)4-cyano-7-hydroxy-thiazolo[4,5-c]pyridine-6-carbonyl]-amino}-acetic acid, {[2,3-dichloro-7-cyano-4-hydroxy-1-(3-methyl-butyl)-1H-pyrrolo[2,3-c]pyridine-5-carbonyl]-amino}-acetic acid, {[7-cyano-4-hydroxy-1-(3-methyl-butyl)-1H-pyrrolo[2,3-c]pyridine-5-carbonyl]-amino}-acetic acid, {[3-chloro-7-cyano-4-hydroxy-1-(3-methyl-butyl)-1H-pyrrolo[2,3-c]pyridine-5-carbonyl]-amino}-acetic acid, {[2,3-dichloro-7-cyano-1-cyclohexylmethyl-4-hydroxy-1H-pyrrolo[2,3-c]pyridine-5-carbonyl]-amino}-acetic acid, {[7-cyano-4-hydroxy-1-cyclohexylmethyl-1H-pyrrolo[2,3-c]pyridine-5-carbonyl]-amino}-acetic acid, [(1-benzyl-3-chloro-4-hydroxy-1H-pyrrolo[2,3-c]pyridine-5-carbonyl)-amino]-acetic acid, [(4-hydroxy-9-methyl-9H-beta-carboline-3-carbonyl)-amino]-acetic acid, [(4-hydroxy-1,9-dimethyl-9H-beta-carboline-3-carbonyl)-amino]-acetic acid, [(4-hydroxy-9-methyl-1-phenyl-9H-beta-carboline-3-carbonyl)-amino]-acetic acid, [(1-cyano-4-hydroxy-9-methyl-9H-beta-carboline-3-carbonyl)-amino]-acetic acid, {[3-bromo-7-cyano-2-(4-fluoro-phenyl)-4-hydroxy-1-phenyl-1H-pyrrolo[2,3-c]pyridine-5-carbonyl]-amino}-acetic acid, {[7-cyano-2-(4-fluoro-phenyl)-4-hydroxy-1-phenyl-1H-pyrrolo[2,3-c]pyridine-5-carbonyl]-amino}-acetic acid, [(4-hydroxy-5-phenyl-5H-pyrido[4,3-b]indole-3-carbonyl)-amino]-acetic acid, [(1-cyano-4-hydroxy-5-phenyl-5H-pyrido[4,3-b]indole-3-carbonyl)-amino]-acetic acid, [(4-hydroxy-1-methyl-5-phenyl-1H-pyrido[4,3-b]indole-3-carbonyl)-amino]-acetic acid, [(1-benzyl-3-chloro-7-cyano-4-hydroxy-1H-pyrrolo[2,3-c]pyridine-5-carbonyl)-amino]-acetic acid, ({[3-cyano-2-(4-fluoro-phenyl)-4-hydroxy-1-phenyl-1H-pyrrolo[2,3-c]pyridine-5-carbonyl]-amino}-acetic acid, {[3-cyano-2-(4-fluoro-phenyl)-4-hydroxy-7-methyl-1-phenyl-1H-pyrrolo[2,3-c]pyridine-5-carbonyl]-amino}-acetic acid, {[3,7-dicyano-2-(4-fluoro-phenyl)-4-hydroxy-1-phenyl-1H-pyrrolo[2,3-c]pyridine-5-carbonyl]-amino}-acetic acid, [(7-cyano-4-hydroxy-1-phenyl-1H-pyrrolo[2,3-c]pyridine-5-carbonyl)-amino]-acetic acid, [(3-chloro-7-cyano-4-hydroxy-1-phenyl-1H-pyrrolo[2,3-c]pyridine-5-carbonyl)-amino]-acetic acid, {[2,3-dibromo-1-(4-fluoro-benzyl)-4-hydroxy-1H-pyrrolo[2,3-c]pyridine-5-carbonyl]-amino}-acetic acid, [(4-hydroxy-1-phenethyl-1H-pyrrolo[2,3-c]pyridine-5-carbonyl)-amino]-acetic acid, ({[2,3-dibromo-7-cyano-1-(4-fluoro-benzyl)-4-hydroxy-1H-pyrrolo[2,3-c]pyridine-5-carbonyl]-amino}-acetic acid, [(3-bromo-7-cyano-4-hydroxy-1-phenyl-1H-pyrrolo[2,3-c]pyridine-5-carbonyl)-amino]-acetic acid, {[7-cyano-1-(4-fluoro-benzyl)-4-hydroxy-1H-pyrrolo[2,3-c]pyridine-5-carbonyl]-amino}-acetic acid, [(3-chloro-7-cyano-4-hydroxy-1-phenethyl-1H-pyrrolo[2,3-c]pyridine-5-carbonyl)-amino]-acetic acid, {[2,3-dibromo-4-hydroxy-1-(1-(S)-phenyl-ethyl)-1H-pyrrolo[2,3-c]pyridine-5-carbonyl]-amino}-acetic acid, {[3-chloro-7-cyano-1-(4-fluoro-benzyl)-4-hydroxy-1H-pyrrolo[2,3-c]pyridine-5-carbonyl]-amino}-acetic acid, [(1-benzyl-2,3-dichloro-7-cyano-4-hydroxy-1H-pyrrolo[2,3-c]pyridine-5-carbonyl)-amino]-acetic acid, {[4-hydroxy-1-(1S-phenyl-ethyl)-1H-pyrrolo[2,3-c]pyridine-5-carbonyl]-amino}-acetic acid, [(2,3-dichloro-7-cyano-4-hydroxy-1-phenyl-1H-pyrrolo[2,3-c]pyridine-5-carbonyl)-amino]-acetic acid, [(2,3-dichloro-7-cyano-4-hydroxy-1-phenethyl-1H-pyrrolo[2,3-c]pyridine-5-carbonyl)-amino]-acetic acid, {[2,3-dichloro-7-cyano-4-hydroxy-1-(1S-phenyl-ethyl)-1H-pyrrolo[2,3-c]pyridine-5-carbonyl]-amino}-acetic acid, [(1-benzyl-3-bromo-7-cyano-4-hydroxy-1H-pyrrolo[2,3-c]pyridine-5-carbonyl)-amino]-acetic acid, {[4-hydroxy-1-(1R-phenyl-ethyl)-1H-pyrrolo[2,3-c]pyridine-5-carbonyl]-amino}-acetic acid, {[4-hydroxy-1-(4-methoxy-benzyl)-1H-pyrrolo[2,3-c]pyridine-5-carbonyl]-amino}-acetic acid, {[7-cyano-4-hydroxy-1-(4-methoxy-benzyl)-1H-pyrrolo[2,3-c]pyridine-5-carbonyl]-amino}-acetic acid, [(1-benzyl-7-cyano-4-hydroxy-3-methyl-1H-pyrrolo[2,3-c]pyridine-5-carbonyl)-amino]-acetic acid, {[2,3-dichloro-7-cyano-4-hydroxy-1-(4-methoxy-benzyl)-1H-pyrrolo[2,3-c]pyridine-5-carbonyl]-amino}-acetic acid, {[2,3-dichloro-7-cyano-4-hydroxy-1-(1R-phenyl-ethyl)-1H-pyrrolo[2,3-c]pyridine-5-carbonyl]-amino}-acetic acid, {[3-chloro-7-cyano-4-hydroxy-1-(4-methoxy-benzyl)-1H-pyrrolo[2,3-c]pyridine-5-carbonyl]-amino}-acetic acid, {[7-cyano-4-hydroxy-1-(4-methoxy-phenyl)-1H-pyrrolo[2,3-c]pyridine-5-carbonyl]-amino}-acetic acid, {[2,3-dichloro-7-cyano-4-hydroxy-1-(4-methoxy-phenyl)-1H-pyrrolo[2,3-c]pyridine-5-carbonyl]-amino}-acetic acid, {[3-chloro-7-cyano-4-hydroxy-1-(4-methoxy-phenyl)-1H-pyrrolo[2,3-c]pyridine-5-carbonyl]-amino}-acetic acid, {[1-(4-fluoro-benzyl)-4-hydroxy-2,3-dimethyl-1H-pyrrolo[2,3-c]pyridine-5-carbonyl]-amino}-acetic acid, {[7-cyano-1-(4-fluoro-phenyl)-4-hydroxy-1H-pyrrolo[2,3-c]pyridine-5-carbonyl]-amino}-acetic acid, {[2,3-dichloro-7-cyano-4-hydroxy-1-(4-fluoro-phenyl)-1H-pyrrolo[2,3-c]pyridine-5-carbonyl]-amino}-acetic acid, {[3-chloro-7-cyano-4-hydroxy-1-(4-fluoro-phenyl)-1H-pyrrolo[2,3-c]pyridine-5-carbonyl]-amino}-acetic acid, {[1-(4-fluoro-benzyl)-4-hydroxy-1H-pyrrolo[2,3-c]pyridine-5-carbonyl]-amino}-acetic acid, [(2-cyano-4-hydroxy-1-phenyl-1H-pyrrolo[2,3-c]pyridine-5-carbonyl)-amino]-acetic acid, {[1-(2-fluoro-benzyl)-4-hydroxy-1H-pyrrolo[2,3-c]pyridine-5-carbonyl]-amino}-acetic acid, {[4-hydroxy-1-(2-methoxy-benzyl)-1H-pyrrolo[2,3-c]pyridine-5-carbonyl]-amino}-acetic acid, {[4-hydroxy-1-(3-methoxy-benzyl)-1H-pyrrolo[2,3-c]pyridine-5-carbonyl]-amino}-acetic acid, {[7-cyano-1-(4-fluoro-phenyl)-4-hydroxy-3-phenyl-1H-pyrrolo[2,3-c]pyridine-5-carbonyl]-amino}-acetic acid, {[7-cyano-1-(2-fluoro-benzyl)-4-hydroxy-1H-pyrrolo[2,3-c]pyridine-5-carbonyl]-amino}-acetic acid (Compound C), {[7-cyano-1-(2-methoxy-benzyl)-4-hydroxy-1H-pyrrolo[2,3-c]pyridine-5-carbonyl]-amino}-acetic acid, {[7-cyano-1-(3-methoxy-benzyl)-4-hydroxy-1H-pyrrolo[2,3-c]pyridine-5-carbonyl]-amino}-acetic acid, {[2-cyano-1-(3-fluoro-benzyl)-4-hydroxy-1H-pyrrolo[2,3-c]pyridine-5-carbonyl]-amino}-acetic acid, {[2,3-dichloro-7-cyano-1-(2-fluoro-benzyl)-4-hydroxy-1H-pyrrolo[2,3-c]pyridine-5-carbonyl]-amino}-acetic acid, {[1-(3-fluoro-benzyl)-4-hydroxy-1H-pyrrolo[2,3-c]pyridine-5-carbonyl]-amino}-acetic acid, {[3-chloro-7-cyano-1-(2-fluoro-benzyl)-4-hydroxy-1H-pyrrolo[2,3-c]pyridine-5-carbonyl]-amino}-acetic acid, {[3-chloro-7-cyano-4-hydroxy-1-(3-methoxy-benzyl)-1H-pyrrolo[2,3-c]pyridine-5-carbonyl]-amino}-acetic acid, {[7-cyano-1-(3-fluoro-benzyl)-4-hydroxy-1H-pyrrolo[2,3-c]pyridine-5-carbonyl]-amino}-acetic acid, {[7-cyano-1-(3,4-difluoro-benzyl)-4-hydroxy-1H-pyrrolo[2,3-c]pyridine-5-carbonyl]-amino}-acetic acid, {[3-chloro-7-cyano-1-(3,4-difluoro-benzyl)-4-hydroxy-1H-pyrrolo[2,3-c]pyridine-5-carbonyl]-amino}-acetic acid, {[2,3-dichloro-7-cyano-1-(3-fluoro-benzyl)-4-hydroxy-1H-pyrrolo[2,3-c]pyridine-5-carbonyl]-amino}-acetic acid, {[3-chloro-7-cyano-1-(3-fluoro-benzyl)-4-hydroxy-1H-pyrrolo[2,3-c]pyridine-5-carbonyl]-amino}-acetic acid, {[2,3-dichloro-7-cyano-1-(3,4-difluoro-benzyl)-4-hydroxy-1H-pyrrolo[2,3-c]pyridine-5-carbonyl]-amino}-acetic acid, [(1-benzyl-2,3-dichloro-7-hydroxy-1H-pyrrolo[3,2-c]pyridine-6-carbonyl)-amino]-acetic acid, [(2-tert-butyl-7-hydroxy-thiazolo[4,5-c]pyridine-6-carbonyl)-amino]-acetic acid, [(2-tert-butyl-7-hydroxy-4-methyl-thiazolo[4,5-c]pyridine-6-carbonyl)-amino]-acetic acid, [(2-tert-butyl-4-cyano-7-hydroxy-thiazolo[4,5-c]pyridine-6-carbonyl)-amino]-acetic acid, [(4-butyl-2-tert-butyl-7-hydroxy-thiazolo[4,5-c]pyridine-6-carbonyl)-amino]-acetic acid, [(2-tert-butyl-7-hydroxy-4-((E)-styryl)-thiazolo[4,5-c]pyridine-6-carbonyl)-amino]-acetic acid, [(2-tert-butyl-7-hydroxy-4-phenyl-thiazolo[4,5-c]pyridine-6-carbonyl)-amino]-acetic acid, [(2-tert-butyl-7-hydroxy-4-phenethyl-thiazolo[4,5-c]pyridine-6-carbonyl)-amino]-acetic acid, [(2-tert-butyl-7-hydroxy-4-isopropylsulfanylmethyl-thiazolo[4,5-c]pyridine-6-carbonyl)-amino]-acetic acid, [(7-hydroxy-2-methyl-4-phenyl-thiazolo[4,5-c]pyridine-6-carbonyl)-amino]-acetic acid, [(7-hydroxy-2-methyl-thiazolo[4,5-c]pyridine-6-carbonyl)-amino]-acetic acid, [(7-hydroxy-2-naphthalen-2-yl-thiazolo[4,5-c]pyridine-6-carbonyl)-amino]-acetic acid, [(7-hydroxy-2-thiophen-2-yl-thiazolo[4,5-c]pyridine-6-carbonyl)-amino]-acetic acid, and [(2-furan-2-yl-7-hydroxy-thiazolo[4,5-c]pyridine-6-carbonyl)-amino]-acetic acid.

In some embodiments, small molecule PHIs may be selected from those disclosed in WO 2004/108121 (U.S. 2005/020487), which can be represented by formula IV:

wherein

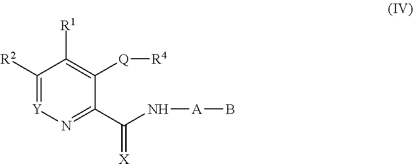

A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₁-C₆)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CFᵣ—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer.

B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, 1, NO₂, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₂)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₂)-alkenyloxycarbonyloxy, (C₂-C₁₂)-alkynyloxycarbonyloxy, carbamoyl, N—(C₁-C₁₂)-alkylcarbamoyl, N.N-di(C₁-C₁₂)-alkylcarbamoyl, N—(C₃-C₈)-cycloalkylcarbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₁-C₆)-aralkyloxy-(C₁-C₁₀)-sub.10)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C.s-ub.10)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C₇-C₁₆)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₂)-aralkylcarbonyl-N—(C₁-C₁₀)-alkylamino, (C₁-C₁₂)alkylcarbonylamino-(C₁-C₈)-alkyl, (C₃-C₈)-cycloalkylcarbonylamino-(C₁-C₈)alkyl, (C₆-C₁₂)-arylcarbonylamino (C₁-C₈)-alkyl, (C₇-C₁₂)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₈)-alkyl, (C₃-C₈)-cycloalkylamlno-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₈)-alkylsulfamoyl, N,N-di-(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₆-C₁₂)-arakysufamnoyl, (C₁-C₁₀)-alkylsulfonamido, N—((C₁-C₁₀)-alkyl)-(C₁-C₁₀)-alkylsulfonamido, (C₁-C₁₆)-aralkylsulfonamido, or N—((C₁-C₁₀)-alkyl-(C₇-C₁₆)-aralkylsulfonamnido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkykcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di-(C₁-C₁₂)-alkylcarbamoyl, N—(C₃-C₈)-cycloalkylcarbamoyl, N—(C₆-C₁₂)-arylcarbamoyl, N—(C₁-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C.-sub.10)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C.-sub.10)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈). cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino (C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₁-C₁₆)-aralkylmercapto, (C₇-C₁₆)-aralkylsulfinyl, or (C₇-C₁₆)-aralkylsulfonyl;

X is O or S;

Q is O, S, NR′, or a bond;

where, if Q is a bond, R⁴ is halogen, nitrile, or trifluoromnethyl;

or where, if Q is O, S, or NR′, R⁴ is hydrogen, (C₁-C₁₀)-alkyl radical, (C₂-C₁₀)-alkenyl radical, (C₃-C₁₀)-alkynyl radical, wherein alkenyl or alkynyl radical contains one or two C—C multiple bonds; unsubstituted fluoroalkyl radical of the formula —[CH₂]ₓ-C_{f}H_{(2f+1-g)}—F_{g}—, (C₁-C₈)-alkoxy-(C₁-C₆)-alkyl radical, (C₁-C₆)alkoxy-(C₁-C₄-alkoxy-(C₁-C₄)-alkyl radical, aryl radical, heteroaryl radical, (C₇-C₁₁)-aralkyl radical, or a radical of the formula Z

—[CH₂]ᵥ—[O]_{w}—[CH₂]ₜ-E (Z)

where

E is a heteroaryl radical, a (C₃-C₈)-cycloalkyl radical, or a phenyl radical of the formula F

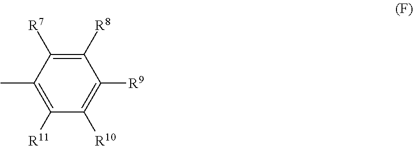

v is 0-6,

w is 0 or 1,

t is 0-3, and

R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓC_{f}H_{(2f+1-g)}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₈)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₆)-alkylcarbamoyl, (C₁-C₆)-alkylcarbonyloxy, phenyl, benzyl, phenoxy, benzyloxy, NR^{Y}R^{Z} wherein R^{Y} and R^{Z} are independently selected from hydrogen, (C₁-C₁₂)-alkyl, (C₁-C₈)-alkoxy-(C₁-C₆)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)-aralkoxy, (C₁-C₁₂)-alkylcarbonyl, (C₃-C₈)-cycloalkylcarbonyl, (C₆-C₁₂)-arylcarbonyl, (C₇-C₁₆)-aralkylcarbonyl; or further wherein R^{Y} and R^{Z} together are —[CH₂]ₕ, in which a CH₂ group can be replaced by O, S, N—(C₁-C₄)-alkylcarbonylimino, or N—(C₁-C₄)-alkoxycarbonylimino, and h is 3 to 7; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R³ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷-R¹¹;

or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted (C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7.

V is N or CR³;

R¹, R² and R³ are identical or different and are hydrogen, hydroxyl, halogen, cyano, trifluoromnethyl, nitro, carboxyl, (C₁-C₂₀)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alky-1, (C₆-C₁₂)-aryloxy, C₇-C₁₆)aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₆)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]_{X}C_{F}H_{(2F+1- g)}—F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₁-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamnoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C.su-b.6)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C.-sub.10)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R

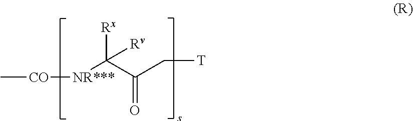

in which

R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong.

s is 1-5,

T is OH, or NR*R**, and R*, R** and R*** are identical or different and are selected from hydrogen, ((C₆-C₁₂)-aryl, (C₇-C₁₆)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₁-C₁₆)-aralkylimino, N—(C₁-C₄)-alkoxy-(C₁-C₆)-alkylimino, and h is from 3 to 7;

carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl) carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C.-sub.10)-alkyl)-carbamoyloxyamino, (C₁-C₁₂)-alkylamino, di-(C₁-C₁₂)-alkylamino, (C₃-C₈)-cycloalkylamino, (C₃-C₁₂)alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₁-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyl-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-al-koxy, (C₃-C₈)-cycloalkyl-(C₁-C₈)-alkoxy-(C₁-C.sub-.6)-alkyl, (C₃-C₈ₐ)-cycloalkyloxy-(C₁-C₈)-alkoxy-(C.sub-.1-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl. (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alky-1, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C.su-b.6)-alkyl)carbamoyl, N-(+)-dehydroabietylcarbamoyl. N—(C₁-C₆)alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₁-C₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alky-N—((C₆-C₁₂)-aryloxy-(C₁-C₁₀)-alky)carbamoyl, N—(C₁-C₁₀)-alkyl-N—((C₇-C₁₆)-aralkyloxy-(C₁-C.-sub.10)-alkyl)-carbamoyl, CON(CH₂)ₕ, in which a CH₂ group can be replaced by, O, S, N—(C₁-C₈)-alkylimino, N—(C₃-C₈)-cycloalkylimnino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy, N—(C₁-C₁₆)-alkyl-N—((C₁-C₁₀)-alkoxy-(C₁-C₁₀)-alkyl)carbamoyloxy, N—(C₁-C₁₀)-alkyl-N—(C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C.-sub.10)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-aroylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₇-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;

or wherein R¹ and R², or R² and R³ form a chain [CH₂]₀, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;

or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;

wherein R¹ and R², or R² and R³ form a carbocyclic or heterocyclic 5- or 6-membered aromatic ring;

or where R¹ and R², or R² and R³, together with the pyridine or pyridazine carrying them, form an optionally substituted heterocyclic ring systems selected from thienopyridines, fiaranopyridines, pyridopyridines, pyrimidinopyridines, imiidazopyridines, thiazolopyridines, oxazolopyridines, quinoline, isoquinoline, and cunnoline; where quinoline, isoquinoline or cinnoline preferably satisfy the formulae IVa, IVb and IVc:

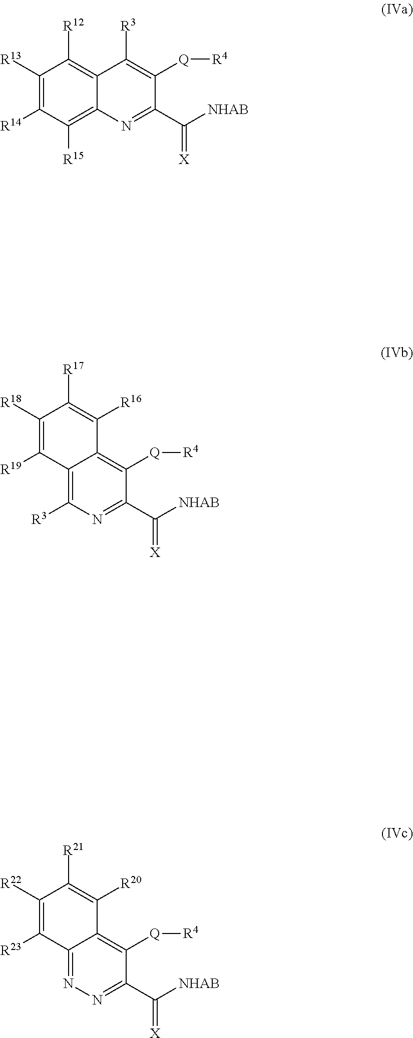

and the substituents R¹² to R²³ in each case independently of each other have the meaning of R¹, R² and R³;

or wherein the radicals R¹ and R²; together with the pyridine carrying them, form a compound of formula IVd:

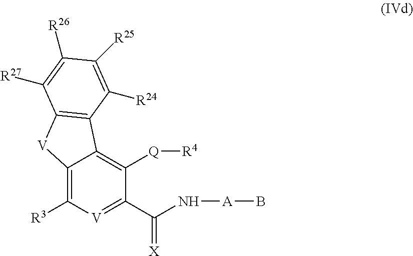

where V is S, O, or NR^{k}, and R^{k} is selected from hydrogen, (C₁-C₆)-alkyl, aryl, or benzyl;

where an aryl radical may be optionally substituted by 1 to 5 substituents as defined above; and

R²⁴, R²⁵, R²⁶ and R²⁷ in each case independently of each other have the meaning of R¹, R² and R³;

f is 1 to 8;

g is 0 or 1 to (2f+1);

x is 0 to 3; and

h is 3 to 7;

including the physiologically active salts and prodrugs derived therefrom.

Exemplary compounds according to formula (NV) are described in European Patent Nos. EP 0650960 and EP 0650961. All compounds listed in EP 0650960 and EP 0650961, in particular, those listed in the compound claims and the final products of the working examples, are hereby incorporated herein by reference.

Additionally, exemplary compounds according to formula (IV) are described in U.S. Pat. No. 5,658,933. All compounds listed in U.S. Pat. No. 5,658,933, in particular, those listed in the compound claims and the final products of the working examples, are hereby incorporated herein by reference.

Additional compounds according to formula (IV) are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated herein by reference.

Exemplary compounds according to formula (IVa) are described in U.S. Pat. Nos. 5,719,164 and 5,726,305. All compounds listed in the foregoing patents, in particular, those listed in the compound claims and the final products of the working examples, are hereby incorporated herein by reference.

Exemplary compounds according to formula (IVb) are described in U.S. Pat. No. 6,093,730. All compounds listed in U.S. Pat. No. 6,093,730, in particular, those listed in the compound claims and the final products of the working examples, are hereby incorporated herein by reference.

Compounds disclosed in WO 2004/108121 (U.S. 2005/020487) can be represented by formula V:

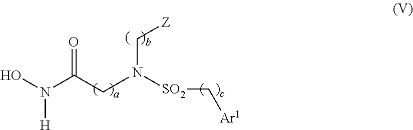

or pharmaceutically acceptable salts thereof, wherein:

a is an integer from 1 to 4;

b is an integer from 0 to 4;

c is an integer from 0 to 4;

Z is selected from the group consisting of (C₃-C₁₀)-cycloalkyl, (C₃-C₁₀)-cycloalkyl independently substituted with one or more Y¹, 3-10 membered heterocycloalkyl and 3-10 membered heterocycloalkyl independently substituted with one or more Y¹; (C₅-C₂₀)-aryl, (C₅-C₂₀)-aryl independently substituted with one or more Y¹, 5-20 membered heteroaryl and 5-20 membered heteroaryl independently substituted with one or more Y¹;

Ar¹ is selected from the group consisting of (C₅-C₂₀)-aryl, (C₅-C₂₀) aryl independently substituted with one or more Y², 5-20 membered heteroaryl and 5-20 membered heteroaryl independently substituted with one or more Y²;

each Y¹ is independently selected from the group consisting of a lipophilic functional group, (C₅-C₂₀)-aryl, (C₆-C₁₂6)-alkaryl, 5-20 membered heteroaryl and 6-26 membered alk-heteroaryl;

each Y² is independently selected from the group consisting of —R′, —OR′, —OR″, —SR′, —SR″, —NR′R′, —NO₂, —CN, -halogen, -trihalomethyl, trihalomethboxy, —C(O)R′, —C(O)OR′, —C(O)NR′R′, —C(O)NR′OR′, —C(NR′R′R)═NOR′, —NR′—C(O)R′, —SO₂R′, SO₂R″, —NR′—SO₂—R′, —NR′—C(O)—NR′R′, tetrazol-5-yl, —NR′—C(O)—OR′, —C(NR′R′)—NR′, —S(O)—R′, S(O)—R″, and —NR′—C(S)—NR′R′; and

each R′ is independently selected from the group consisting of —H, (C₁-C₈)-alkyl, (C₂-C₈)-alkenyl, and (C₂-C₈)-alkynyl; and.

each R″ is independently selected from the group consisting of (C₅-C₂₀)-aryl and (C₅-C₂₀)-aryl independently substituted with one or more OR′, —SR′, —NR′R′, —NO₂, —CN, halogen or trihalomethyl groups,

or wherein c is 0 and Ar¹ is an N′ substituted urea-aryl, the compound has the structural formula (Va):

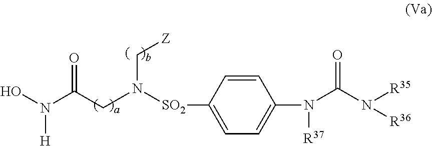

or pharmaceutically acceptable salts thereof, wherein:

a, b, and Z are as defined above; and

R³⁵ and R³⁶ are each independently selected from the group consisting of hydrogen, (C₁-C₈)-alkyl, (C₂-C₈)-alkenyl, (C₂-C₈)-alkynyl, (C₃-C₁₀)-cycloalkyl, (C₅-C₂₀)-aryl, (C₅-C₂₀)-substituted aryl, (C₆-C₂6)-alkaryl, (C₆-C₂6)-substituted alkaryl, 5-20 membered heteroaryl, 5-20 membered substituted heteroaryl, 6-26 membered alk-heteroaryl, and 6-26 membered substituted alk-heteroaryl; and

R³⁷ is independently selected from the group consisting of hydrogen, (C₁-C₈)-alkyl, (C₂-C₈)-alkenyl, and (C₂-C₈)-alkynyl.

Additional compounds disclosed in WO 2003/053997 (U.S. 2003/153503) can be represented by formula VI:

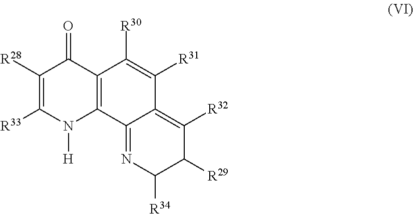

wherein

R²⁸ is hydrogen, nitro, amino, cyano, halogen, (C₁-C₄)-alkyl, carboxy or a metabolically labile ester derivative thereof; (C₁-C₄)-alkylamino, di-(C₁-C₄)-alkylamino, (C₁-C₆)-alkoxycarbonyl, (C₂-C₄)-alkanoyl, hydroxy-(C₁-C₄)-alkyl, carbamoyl, N—(C₁-C₄)-alkylcarbamoyl, (C₁-C₄)-alkylthio, (C₁-C₄)-alkylsulfinyl, (C₁-C₄)-alkylsulfonyl, phenylthio, phenylsulfinyl, phenylsulfonyl, said phenyl or phenyl groups being optionally substituted with 1 to 4 identical or different halogen, (C₁-C₄)-alkyoxy, (C₁-C₄)-alkyl, cyano, hydroxy, trifluoromethyl, fluoro-(C₁-C₄)-alkylthio, fluoro-(C₁-C₄)-alkylsulfinyl, fluoro-(C₁-C₄)-alkylsulfonyl, (C₁-C₄)-alkoxy-(C₂-C₄)-alkoxycarbonyl, N,N-di-[(C₁-C₄)-alkyl]carbamoyl-(C₁-C₄)-alkoxycarbonyl, (C₁-C₄)-alkylamino-(C₂-C₄-alkoxycarbonyl, di-(C₁-C₄)-alkylamino-(C₂-C₄)-alkoxycarbonyl, (C₁-C₄)-alkoxy-(C₂-C₄)-alkoxy-(C₂-C₄)-alkoxycarbonyl, (C₂-C₄)-alkanoyloxy-(C₁-C₄)-alkyl, or N-[amino-(C₂-C₅)-alkyl]-carbamoyl;

R²⁹ is hydrogen, hydroxy, amino, cyano, halogen, (C₁-C₄)-alkyl, carboxy or metabolically labile ester derivative thereof, (C₁-C₄)-alkylamino, di-(C₁-C₄)-alkylamino, (C₁-C₆)-alkoxycarbonyl, (C₂-C₄)-alkanoyl, (C₁-C₄)-alkoxy, carboxy-(C₁-C₄)-alkoxy, (C₁-C₄)-alkoxycarbonyl-(C₁-C₄)-alkoxy, carbamoyl, N—(C₁-C₈)-alkylcarbamoyl, N,N-di-(C₁-C₈)-alkylcarbamoyl, N-[amino-(C₂-C₈)-alkyl]-carbamoyl, N—[(C₁-C₄)-alkylamino-(C₁-C₈)-alkyl]-carbamoyl, N-[di-(C₁-C₄)-alkylamino-(C₁-C₈)alkyl)]-carbamoyl, N-cyclohexylcarbamoyl, N-[cyclopentyl]-carbamoyl, N—(C₁-C₄)-alkylcyclohexylcarbamoyl, N—(C₁-C₄)-alkylcyclopentylcarbamoyl, N-phenylcarbamoyl, N—(C₁-C₄)-alkyl-N-phenylcarbamoyl, N,N-diphenylcarbamoyl, N-[phenyl-(C₁-C₄)-alkyl]-carbamoyl, N—(C₁-C₄)alkyl-N-[phenyl-(C₁-C₄)-alkyl]-carbamoyl, or N,N-di-[phenyl-(C₁-C₄)-alkyl]-carbamoyl, said phenyl or phenyl groups being optionally substituted with

1 to 4 identical or different halogen, (C₁-C₄)-alkyoxy, (C₁-C₄-alkyl, cyano, hydroxy, trifluoromethyl, N—[(C₂-C₄)-alkanoyl]-carbamoyl, N—[(C₁-C₄)-alkoxycarbanyl]-carbamoyl, N-[fluoro-(C₂-C₆)-alkyl]-carbamoyl, N,N-[fluoro-(C₂-C₆)-alkyl]-N—(C₁-C₄)-alkylcarbamoyl, N,N-[di-fluoro-(C₂-C₆)-alkyl]carbamoyl, pyrrolidin-1-ylcarbonyl, piperidinocarbonyl, piperazin-1-ylcarbonyl, morpholinocarbonyl, wherein the heterocyclic group, is optionally substituted with 1 to 4, (C₁-G)-alkyl, benzyl, 1,2,3,4-tetrahydro-isoquinolin-2-ylcarbonyl, N,N-[di-(C₁-C₄)-alkyl]-thiocarbamoyl, N—(C₂-C₄)-alkanoylamino, or N—[(C₁-C₄)-alkoxycarbonyl]-amino;

R³⁰ is hydrogen, (C₁-C₄)-alkyl, (C₂-C₄)-alkoxy, halo, nitro, hydroxy, fluoro-(C₁-C₄)-alkyl, or pyridinyl;

R³¹ is hydrogen, (C₁-C₄)-alkyl, (C₂-C₄)-alkoxy, halo, nitro, hydroxy, fluoro-(C₁-C₄)-alkyl, pyridinyl, or methoxy;

R³² is hydrogen, hydroxy, amino, (C₁-C₄)-alkylamino, di-(C₁-C₄)-alkylamino, halo, (C₁-C₄-alkoxy-(C₂-C₄)-alkoxy, fluoro-(C₁-C₆)-alkoxy, pyrrolidin-1-yl, piperidino, piperazin-1-yl, or morpholino, wherein the heterocyclic group is optionally substituted with 1 to 4 identical or different (C₁-C₄)-alkyl or benzyl; and

R³³ and R³⁴ are individually selected from hydrogen, (C₁-C₄)-alkyl, and (C₁-C₄)-alkoxy;

including pharmaceutically-acceptable salts and pro-drugs derived therefrom.

Exemplary compounds disclosed in WO 2005/034929, WO 2005/007192, WO 2004/108121 (U.S. 2005/020487), WO 2003/053997 (U.S. 2003/153503), and WO 2003/049686 (U.S. 2003/176317) include [(7-chlor-3-hydroxy-quinoline-2-carbonyl)-amino]-acetic acid; [(1-chloro-4-hydroxy-isoquinoline-3-carbonyl)-amino]-acetic acid; 4-oxo-1,4-dihydro-[1,10]phenanthroline-3-carboxylic acid, [(3-hydroxy-6-isopropoxy-quinoline-2-carbonyl)-amino]-acetic acid; [(1-bromo-4-hydroxy-7-trifluoromethyl-isoquinoline-3-carbonyl)-amino]-acetic acid; 4-hydroxy-5-methoxy-[1,10]phenanthroline-3-carboxylic acid ethyl ether; [(7-chloro-3-hydroxy-quinoline-2-carbonyl)-amino]-acetic acid, sodium salt; 3-{[4-(3,3-dibenzyl-ureido)-benzenesulfonyl]-[2-(4-methoxy-phenyl)-ethyl]-amino}-N-hydroxy-propionamide; [(4-hydroxy-7-phenylsulfanyl-isoquinoline-3-carbonyl)-amino]-acetic acid; [(4-hydroxy-6-phenylsulfanyl-isoquinoline-3-carbonyl)-amino]-acetic acid; [l-chloro-4-hydroxy-7-phenoxy-isoquinoline-3-carbonyl)-amino]-acetic acid; [(1-bromo-4-hydroxy-7-phenoxy-isoquinoline-3-carbonyl-amino]-acetic acid; [(4-hydroxy-7-phenoxy-isoquinoline-3-carbonyl)-amino]-acetic acid; [(1-chloro-4-hydroxy-isoquinoline-3-carbonyl)-amino]-acetic acid, 3-carboxy-5-hydroxy-4-oxo-3,4-dihydro-phenanthroline; 3-carboxy-5-methoxy-4-oxo-3,4-dihydro-1,10-phenanthroline; 5-methoxy-4-oxo-1,4-dihydro-[1,10]phenanthroline-3-carboxylic acid ethyl ester, S-methoxy-4-oxo-1,4-dihydro-[1,10]phenanthroline-3-carboxylic acid; 3-carboxy-8-hydroxy-4-oxo-3,4-dihydro-1,10-phenanthroline; [(3-hydroxy-pyridine-2-carbonyl)-amino]-acetic acid; [(3-methoxy-pyridine-2-carbonyl)-amino]-acetic acid; 3-methoxy-pyridine-2-carboxylic acid N-(((hexadecyloxy)-carbonyl)-methyl)-amide hydrochloride; 3-methoxypyridine-2-carboxylic acid N-(((1-octyloxy)-carbonyl)-methyl)-amide; 3-methoxypyridine-2-carboxylic acid N-(((hexyloxy)-carbonyl)-methyl)-amide; 3-methoxypyridine-2-carboxylic acid N-(((butyloxy)-carbonyl)-methyl)-amide; 3-methoxypyridine-2-carboxylic acid N-(((2-nonyloxy)-carbonyl)-methyl)-amide racemate; 3-methoxypyridine-2-carboxylic acid N-(((heptyloxy)-carbonyl)-methyl)-amide; 3-benzyloxypyridine-2-carboxylic acid N-(((octyloxy)-carbonyl)-methyl)-amide; 3-benzyloxypyridine-2-carboxylic acid N-(((butyloxy)-carbonyl)-methyl)-amide; 5-(((3-(1-butyloxy)-propyl)-amino)-carbonyl)-3-methoxypyridine-2-carboxylic acid N-((benzyloxycarbonyl)-methyl)-amide; 5-(((3-(1-butyloxy)-propyl)-amino)-carbonyl)-3-methoxypyridine-2-carboxylic acid N-(((1-butyloxy)carbonyl)-methyl)-amide; 5-(((3-lauryloxy)-propyl)amino)-carbonyl)-3-methoxypyridine-2-carboxylic acid N-(((benzyloxy)-carbonyl)-methyl)-amide; N-((6-(1-butyloxy)-3-hydroxyquinolin-2-yl)-carbonyl)-glycine; [(3-hydroxy-6-trifluoromethoxy-quinoline-2-carbonyl)-amino]-acetic acid; N-((6-chloro-3-hydroxyquinolin-2-yl)-carbonyl)-glycine; N-((7-chloro-3-hydroxyquinolin-2-yl)-carbonyl)-glycine; [(6-chloro-3-hydroxy-quinoline-2-carbonyl)-amino]-acetic acid; N-((1-chloro-4-hydroxy-7-(2-propyloxy)isoquinolin-3-yl)-carbonyl)-glycine-; N-((1-chloro-4-hydroxy-6-(2-propyloxy)isoquinolin-3-yl)-carbonyl)-glycine; N-((1-chloro-4-hydroxy-isoquinoline-3-carbonyl)-amino)-acetic acid; N-((1-chloro-4-hydroxy-7-methoxyisoquinolin-3-yl-carbonyl)-glycine; N-((1-chloro-4-hydroxy-6-methoxyisoquinolin-3-yl)-carbonyl)-glycine; N-((7-butyloxy)-1-chloro-4-hydroxyisoquinolin-3-yl)-carbonyl)-glycine; N-((6-benzyloxy-1-chloro-1-hydroxy-isoquinoline-3-carbonyl)-amino)-acetic acid; ((7-benzyloxy-1-chloro-4-hydroxy-isoquinoline-3-carbonyl)-amino)-acetic acid methyl ester N-((7-benzyloxy-1-chloro-4-hydroxy-isoquinoline-3-carbonyl)-amino)-acetic acid; N-((8-chloro-4-hydroxyisoquinolin-3-yl)-carbonyl)-glycine; N-((7-butoxy-4-hydroxy-isoquinoline-3-carbonyl)-amino)-acetic acid; 6-cyclohexyl-1-hydroxy-4-methyl-1H-pyridin-2-one; 7-(4-methyl-piperazin-1-ylmethyl)-5-phenylsulfanylmethyl-quinolin-8-ol; 4-nitro-quinolin-8-ol; 5-butoxymethyl-quinolin-8-ol; 3-({4-[3-(4-chloro-phenyl)-ureido]-benzenesulfonyl)-[2-(4-methoxy-phenyl)-ethyl]-amino}-N-hydroxy-propionamide; 3-{{4-[3-(1,2-diphenyl-ethyl)-ureido]-benzenesulfonyl}-[2-(4-methoxy-phenyl)-ethyl]-amino}-N-hydroxy-propionamide; and pharmaceutically acceptable salts; esters; and prodrugs thereof.

In other embodiments, PHIs are selected from quinazolinone compounds as disclosed for example in U.S. 2010/0204226, which are represented by formula VII:

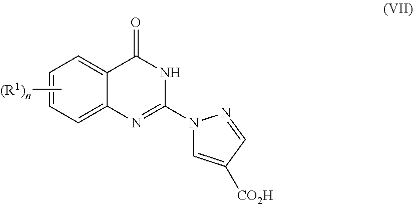

wherein:

n is 0-3;

R¹ is a member independently selected from the group consisting of halo, —C₁₋₄alkyl, —C₁₋₄alkynyl, —C₁₋₄alkenyl optionally substituted with halo, —CF₃, —OCF₃, —SCF₃, S(O)CF₃, —C(O)—R^{c}, —C(O)N—R^{c}, —OH, —NO₂, —CN, —OC₁₋₄alkyl, —C₁₋₄alkyl, —S(O)—C₁₋₄alkyl, —SO₂, —C₁₋₄alkyl, —S—R^{c}, —S(O)₂—R^{c}, —SO₂—R^{c}, —SO₂N—R^{c}, —O—R^{c}NR³R^{b}, 2,3-dihydro-benzo[1,4]dioxine, benzo[1,3]dioxole, 1H-indole, benzyl, biphenyl optionally substituted with one or more R^{d} members, benzyloxy optionally substituted with one or more R^{d} members, phenyl or monocyclic heteroaryl optionally substituted with one or more R^{d} members, —C₃₋₈cycloalkyl optionally substituted with one or more R^{d} members, —C₃₋₈heterocycloalkyl optionally substituted with one or more R members, and two adjacent R¹ groups may be joined to form an optionally substituted 3-8 member ring optionally containing one or more O, S or N;

R^{a} and R^{b} are independently selected from the group consisting of H, C₁₋₄alkyl, —C(O)C₁₋₄alkyl, —C(O)—R^{c}, —C(O)NH—R^{c}, —SO₂—R^{c}, —SO₂—C₁₋₄alkyl, phenyl optionally substituted with R^{d}, benzyl optionally substituted with R^{d} or monocyclic heteroaryl ring optionally substituted with R^{d}; or

R^{a} and R^{b} can be taken together with the nitrogen to which they are attached to form an optionally substituted monocyclic heterocycloalkyl ring containing one or more O, S or N;

R^{c} is a member independently selected from the group consisting of —C₃₋₈cycloalkyl, —C₃₋₈heterocycloalkyl, biphenyl, phenyl optionally substituted with one or more R^{d} members, benzyl optionally substituted with R^{d}, naphthyl, indanyl, 5,6,7,8-tetrahydro-naphthyl, and pyridyl optionally substituted with one or more R^{d} members;

R^{d} is a member independently selected from the group consisting of —H, halo, —OH, —C₁₋₄alkyl, —SO₂—C₁₋₄alkyl, —CN, or —CF₃, —OCF₃, —OC₁₋₄alkyl, —C(O)NH₂, —O— phenyl, and —O-benzyl; and

enantiomers, diastereomers, racemates, and pharmaceutically acceptable salts thereof.

Some embodiments of the compounds of formula VII have any one or more of the following:

R¹ is a member independently selected from the group consisting of halo, —C₁₋₄alkyl, —OCF₃, —CF₃, —OH, —NO₂, —CN, —OC₁₋₄alkyl, —SC₁₋₄alkyl, —S(O)—C₁₋₄alkyl, —SO₂—C₁₋₄alkyl, —S—R^{c}, —S(O)—R^{c}, —SO₂—R^{c}, —O—R^{c}, —NR^{a}R^{b}, benzyloxy optionally substituted with R^{d}, phenyl or monocyclic heteroaryl optionally substituted with one or more R^{d} members, and —C₃₋₈cycloalkyl optionally containing O, S or N wherein said —C₃₋₈cycloalkyl is optionally substituted with R^{d};

two adjacent R¹ groups are joined to form an aromatic 3-8 membered ring optionally containing one or more O, S or N;

two adjacent R¹ groups are joined to form an optionally substituted 3-8 membered ring containing one or more O, S or N;

n is 1, 2 or 3;

—R^{a}R^{b} is a member independently selected from the group consisting of —H, —CH₃, —CH₂CH₃, benzoyl, 2,6-dimethylbenzoyl, acetyl, —C(O)NH-phenyl, benzenesulfonyl, methanesulfonyl, benzyl, 2-methylbenzyl, 2-chlorobenzyl, 2,6-dimethylbenzyl, 2,6-difluorobenzyl, 2-cyanobenzyl, 3-cyanobenzyl, 3-carbamoyl-benzyl, 2,6-dichlorobenzyl, 3-chlorobenzyl, and 4-methylbenzyl;

R^{a} and R^{b} can be taken together with the nitrogen to which they are attached to form an optionally substituted N-methylpiperazin-1-yl, 3,4-dihydro-1H-isoquinolin-2-yl, piperidinyl, morpholin-4-yl, and pyrrolidinyl;

R^{c} is a member independently selected from the group consisting of phenyl, cyclohexyl, 4-tert-butyl-phenyl, 3,4-dimethoxy-phenyl, 2,6-dimethyl-phenyl, 3,4,5-trimethoxy-phenyl, naphthalen-1-yl, 3-chloro-phenyl, 4-chloro-phenyl, 3-methoxy-phenyl, 4-fluoro-phenyl, 2-fluoro-phenyl, 3-fluoro-phenyl, 3,5-di-tert-butyl-phenyl, 4-oxo-6-m-tolyl, 4-oxo-6-o-tolyl, 2,6-dichloro-phenyl, 2,4-dichloro-phenyl, 2,5-dichloro-phenyl, 4-methoxy-phenyl, 2,6-dimethyl-phenyl, naphthalen-2-yl, 5,6,7,8-tetrahydro-naphthalen-1-yl, 4-chloro-phenyl, p-tolyl, indan-5-yl, 2,3-dichloro-phenyl, and pyridin-3-yl;

R^{d} is a member independently selected from the group consisting of —H, chloro, fluoro, bromo, iodo, —C₁₋₄alkyl, —CF₃, —OCF₃, —OC₁₋₄alkyl, phenyl, —O-phenyl, or —O-benzyl; and

R¹ is independently selected from the group consisting of chloro, fluoro, bromo, iodo, —NO₂, —OH, —CF₃, —CH₃, —CH₂CH₃, —CH₂CH₂CH₃, —OCF₃, —OCH₃, —OCH₂CH₃, —SCH₃, —SCF₃, —S(O)CF₃, —SO₂CH₃, —NH₂, —N(CH₃)₂, —NH(CH₂CH₃), cyano, isopropoxy, isopropyl, sec-butyl, tert-butyl, ethynyl, 1-chloro-vinyl, 4-methyl-piperazinyl, morpholin-4-yl, pyrrolidinyl, pyrrolidine-1-carbonyl, piperidinyl, phenyl, benzyl, biphenyl, tolyl, phenoxy, cyclopropyl, cyclohexyl, phenylsulfanyl, 3,4-dimethoxy-phenylsulfanyl, 4-tert-butyl-phenylsulfanyl, 7-piperidinyl, 2,6-dimethyl-phenoxy, 3,4,5-trimethoxy-phenoxy, naphthalen-1-yloxy, naphthalen-2-yloxy, 5,6,7,8-tetrahydro-naphthalen-1-yloxy, indan-5-yloxy, 3-chlorophenoxy, 4-chlorophenoxy, 2,3-dichloro-phenoxy, 3-methoxy-phenoxy, 4-fluorophenoxy, 2-fluorophenoxy, 3-fluorophenoxy, 3,5-di-tert-butyl-phenoxy, 3-methylphenoxy, 2,6-dichloro-phenoxy, 2,5-dichlorophenoxy, 4-methoxyphenoxy, pyridin-3-yloxy, tetrahydro-pyran-4-yl, 3,4-dihydro-1H-isoquinolin-2-yl, 7-bromo-3,4-dihydro-1H-isoquinolin-2-yl, 3-methoxyphenyl-piperidinyl, and benzenesulfonyl.

In still other embodiments, PHIs are selected from benzoimidazole compounds as disclosed for example in U.S. 2011/0046132, which is expressly incorporated herein by reference in its entirety. In representative examples, the benzoimidazole compounds are represented by formula VIII:

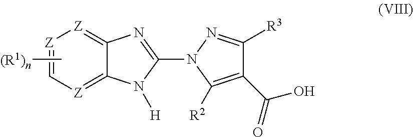

wherein:

n is 2-4;

each R¹ is independently selected from H, halo, —C₁₋₄alkyl, —C₃₋₈cycloalkyl-C₁₋₄perhaloalkyl, trifluoroC₁₋₄alkoxy, —OH, —NO₂, —CN, CO₂H, —OC₁₋₄alkyl, —SC₁₋₄alkyl, —S(C₁₋₄alkyl)-R^{c}, —S(O)₂(C₁₋₄alkyl)-R^{c}, —S(O)—C₁₋₄alkyl, —SO₂—C₁₋₄alkyl, —S—R^{c}, —S(O)—R^{c}, —SO₂—R^{c}, —SO₂—NH—R^{c}, —O—R^{c}, —CH₂—O—R^{c}, —C(O)NH—R^{c}, —NR^{a}R^{b}, benzyloxy optionally substituted with R^{d}, phenyl or monocyclic heteroaryl optionally substituted with R^{d}, —C₃₋₈cycloalkyl optionally containing one or more O, S or N wherein said —C₃₋₈cycloalkyl is optionally substituted with R^{d}, and two adjacent R¹ groups may be joined to form an optionally substituted 3-8 member ring optionally containing one or more O, S or N;

R^{a} and R^{b} are each independently H, C₁₋₄alkyl, —C(O)C₁₋₄alkyl, —C(O)—R^{c}, —C(O)CH₂—R^{c}, C₁₋₄alkyl-R, —SO₂—R^{c}, —SO₂—C₁₋₄alkyl, phenyl optionally substituted with R^{d}, benzyl optionally substituted with R^{d} or monocyclic heteroaryl ring optionally substituted with R^{d}; or

R^{a} and R^{b} can be taken together with the nitrogen to which they are attached to form an optionally substituted monocyclic heterocycloalkyl ring optionally containing one or more heteroatoms;

R^{c} is —C₃₋₈cycloalkyl, phenyl optionally substituted with R^{d}, benzyl optionally substituted with R^{d}, or a monocyclic heteroaryl ring optionally substituted with R^{d};

R^{d} is independently —H, halo, —OH, —C₁₋₄alkyl or —C₁₋₄ perhaloalkyl, trifluoroC₁₋₄alkoxy, —OC₁₋₄alkyl, —O-phenyl, or —O-benzyl;

R^{e} is —C₃₋₈heterocycloalkyl optionally containing one or more O, S or N; R² and R³ are both H, —CF₃, or C₁₋₃alkyl; each Z is C or N, provided that no more than two Z's can simultaneously be N; and

enantiomers, diastereomers, racemates, and pharmaceutically acceptable salts thereof.

Some embodiments of the compounds of formula VIII have any one or more of the following:

R² and R³ are each —H;

R¹ is independently selected from the group consisting of H, halo, —CF₃, —OCF₃, phenyl (optionally substituted or unsubstituted with up to three —CF₃, halo, —OH, C₁₋₄alkyl, C₁₋₄alkoxy, and —OCF₃), phenoxy (optionally substituted or unsubstituted with up to three halo, C₁₋₄ alkyl, C₁₋₄ alkoxy, and —OCF₃), benzyloxy-phenyl (optionally substituted or unsubstituted with up to three halo), benzyloxy, benzyloxymethyl, phenylsulfanyl (optionally substituted or unsubstituted with up to three —C₁₋₄alkyl, halo, —CF₃, —OCF₃, and —C₁₋₄alkoxy), benzylsulfanyl (optionally substituted or unsubstituted with up to three halo, C₁₋₄alkyl, C₃₋₈cycloalkylmethyl, —CF₃, and —OCF₃) phenethylsulfanyl, benzenesulfonyl (optionally substituted or unsubstituted with up to three C₁₋₄alkyl, C₁₋₄alkoxy, halo, —CF₃, and —OCF₃), phenylmethanesulfonyl (optionally substituted or unsubstituted with up to three C₁₋₄alkyl, C₁₋₄alkoxy, halo, C₃₋₈cycloalkylmethyl, —CF₃, and —OCF₃, phenyl-ethanesulfonyl, benzenesulfinyl, cyano-biphenyl-4-ylmethylsulfanyl, cyano-biphenyl-4-ylmethanesulfonyl, phenylcarbamoyl, benzylcarbamoyl, benzylamino, phenylsulfamoyl, phenylamino, benzoylamino, and benzenesulfonylamino;

two adjacent R¹ groups are joined to form an optionally substituted 3-8 membered ring containing one or more O, S or N;

the optionally substituted 3-8 membered ring is aromatic; and

each R¹ is independently selected from H, halo, —C₁₋₄alkyl, —CF₃, —C₃₋₈cycloalkyl, —OCF₃, —C₁₋₄alkylsulfonyl, —C₁₋₄alkylsulfinyl, —C₁₋₄alkylsulfanyl, —NO₂, —NH₂, —NH—C₁₋₄alkyl, —NH—SO₂—C₃₋₈cycloalkyl, —NH—SO₂—C₁₋₄alkyl, —NH—C(O)—C₁₋₄alkyl, —CN, —CO₂H, —OC₁₋₄alkyl, —NH—(CH₂)₂-morpholine, —NH(CO)CH-morpholine, —NHC(O)—CH₂-piperidine, —NHC(O)—CH₂—(N-methylpiperazine), —NH—C₁₋₄ alkyl-morpholine, —S—(CH₂)₂-morpholine, —C(O)—NH-morpholine, pyrrolidine, piperidine, and morpholine.

Other embodiments of PHIs are suitably selected from triazolopyridine compounds as disclosed for example in U.S. 2011/0077267, which are represented by formula IX, or a pharmaceutically acceptable salt thereof, or a solvate thereof:

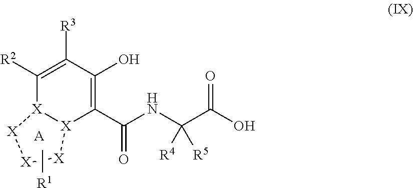

wherein the partial structural formula:

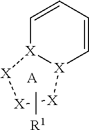

is a group represented by any of the following formulas:

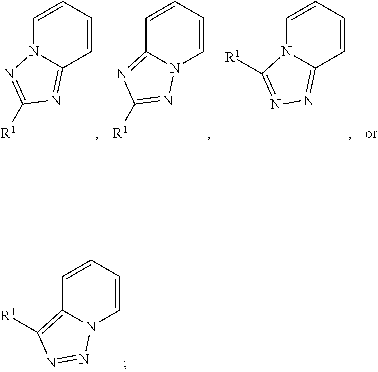

R¹ is (1) a hydrogen atom, (2) a C₁₋₆ alkyl group, (3) a C₆₋₁₄ aryl group, (4) a C₃₋₈ cycloalkyl group, (5) a C₆₋₁₄ aryl-C₁₋₆ alkyl group, or (6) a C₃₋₈ cycloalkyl-C₁₋₆ alkyl group;

R² is (1) a hydrogen atom, (2) a C₁₋₁₀ alkyl group, (3) a C₆₋₁₄ aryl group optionally substituted by the same or different 1 to 5 substituents selected from the following group B, (4) a C₃₋₈ cycloalkyl group optionally substituted by the same or different 1 to 5 substituents selected from the following group B, (5) a C₃₋₈ cycloalkenyl group optionally substituted by the same or different 1 to 5 substituents selected from the following group B, (6) a heteroaryl group optionally substituted by the same or different 1 to 5 substituents selected from the following group B (wherein the heteroaryl has, besides carbon atom, 1 to 6 hetero atoms selected from nitrogen atom, oxygen atom and sulfur atom), (7) a C₆₋₁₄ aryl-C₁₋₆ alkyl group (wherein C₆₋₁₄ aryl is optionally substituted by the same or different 1 to 5 substituents selected from the following group B), or (8) a C₃₋₈ cycloalkyl-C₁₋₆ alkyl group (wherein C₃₋₈ cycloalkyl is optionally substituted by the same or different 1 to 5 substituents selected from the following group B); and

R³ is (1) a hydrogen atom, (2) a halogen atom, (3) a C₁₋₆ alkyl group, (4) a C₆₋₁₄ aryl group, (5) a C₃₋₈ cycloalkyl group, or (6) a C₆₋₁₄ aryl-C₁₋₆ alkyl group; and R⁴ and R⁵ are each independently (1) a hydrogen atom, or (2) a C₁₋₆ alkyl group, group B: (a) a halogen atom, (b) a C₁₋₆ alkyl group, (c) a C₃₋₈ cycloalkyl group, (d) a cyano group, and (e) a halo-C₁₋₆ alkyl group.

Other embodiments of PHIs are suitably selected from the pyrimidinyl compounds as disclosed in U.S. Pat. No. 8,324,208, which are represented by formula XI

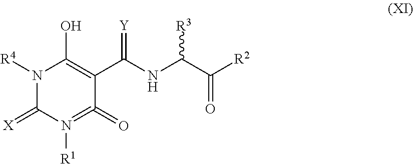

R1 and R4 are each independently selected from the group consisting of hydrogen, —NR5R6, C1-C10 alkyl, C2-C10 alkenyl, C2-C10 alkynyl, C3-C8 cycloalkyl, C3-C8 cycloalkyl-C1-C10 alkyl, C5-C8 cycloalkenyl, C5-C8 cycloalkenyl-C1-C10 alkyl, C3-C8 heterocycloalkyl, C3-C8 heterocycloalkyl-C1-C10 alkyl, aryl, aryl-C1-C10 alkyl, heteroaryl and heteroaryl-C1-C10 alkyl;

R2 is —NR7R8 or —OR9;

R3 is H or C1-C4 alkyl;

where R5 and R6 are each independently selected from the group consisting of hydrogen, C1-C10 alkyl, C3-C8 cycloalkyl, C3-C8 cycloalkyl-C1-C10 alkyl, C3-C8 heterocycloalkyl, C3-C8 heterocycloalkyl-C1-C10 alkyl, aryl, aryl-C1-C10 alkyl, heteroaryl, heteroaryl-C1-C10 alkyl, —C(O)C1-C4alkyl, —C(O)C3-C6 cycloalkyl, —C(O)C3-C6 heterocycloalkyl, —C(O)aryl, —C(O) heteroaryl and —S(O)2C1-C4alkyl, or, when R5 and R6 are attached to the same nitrogen, R5 and R6 taken together with the nitrogen to which they are attached form a 5- or 6- or 7-membered saturated ring optionally containing one other heteroatom selected from oxygen, nitrogen and sulphur,

R7 and R8 are each independently selected from the group consisting of hydrogen, C1-C10 alkyl, C2-C10 alkenyl, C2-C10 alkynyl, C3-C8 cycloalkyl, C3-C8 heterocycloalkyl, aryl and heteroaryl, and R9 is H or a cation, or C1-C10 alkyl which is unsubstituted or substituted with one or more substituents, suitably from 1 to 6 substituents, suitably from 1 to 3 substituents, independently selected from the group consisting of C3-C6 cycloalkyl, heterocycloalkyl, aryl and heteroaryl;

X is O or S; and

Y is O or S;

where any carbon or heteroatom of R1, R2, R3, R4, R5, R6, R7, R8, R9 is unsubstituted or, where possible, is substituted with one or more substituents, suitably from 1 to 6 substituents, suitably from 1 to 3 substituents, independently selected from C1-C6 alkyl, C1-C6 haloalkyl, halogen, —OR10, —NR5R6, oxo, cyano, nitro, —C(O)R10, —C(O)OR10, —SR10, —S(O)R10, —S(O)2R10, —NR5R6, —CONR5R6, —N(R5)C(O)R10, —N(R5)C(O)OR10, —OC(O)NR5R6, —N(R5)C(O)NR5R6, —SO₂NR5R6, —N(R5)SO₂R10, C2-C10 alkenyl, C2-C10 alkynyl, C3-C6 cycloalkyl, C3-C6 heterocycloalkyl, aryl, C1-C6 alkyl-aryl, heteroaryl and C1-C6 alkyl-heteroaryl, wherein R5 and R6 are the same as defined above and R10 is selected from hydrogen, C1-C10alkyl, C2-C10alkenyl, C2-C10 alkynyl, —C(O)C1-C4 alkyl, —C(O)aryl, —C(O) heteroaryl, —C(O)C3-C6 cycloalkyl, —C(O)C3-C6 heterocycloalkyl, —S(O)2C1-C4 alkyl, C3-C8 cycloalkyl, C3-C8 heterocycloalkyl, C6-C14 aryl, aryl-C1-C10 alkyl, heteroaryl and heteroaryl-C1-C10 alkyl; and/or a pharmaceutically acceptable salt or solvate thereof.

Still other embodiments of PHIs are suitably selected from the pyridine compounds as disclosed in U.S. Pat. No. 7,811,595, which is represented by formula XIl:

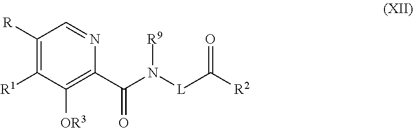

wherein R and R1 are each independently: i) hydrogen; ii) substituted or unsubstituted phenyl; or iii) substituted or unsubstituted heteroaryl; said substitutions being: i) C1-C4 linear, branched, or cyclic alkyl; ii) C1-C4 linear, branched, or cyclic alkoxy; iii) C1-C4 linear, branched, or cyclic haloalkyl; iv) halogen; v) —CN; vi) —NHC(O)R4 vii) —C(O)NR5aR5b; or viii) heteroaryl; or ix) two substitutions are taken together to form a fused ring having from S to 7 atoms;

R4 is C1-C4 linear, branched, or cyclic alkyl;

R5a and R5b are each independently: i) hydrogen; ii) C1-C4 linear, branched, or cyclic alkyl; or iii) R5a and R5b are taken together to form a ring having from 3 to 7 atoms;

R2 is: i) —OR6; or ii) —NR7aR7b;

R6 is hydrogen or C1-C4 linear, branched, or cyclic alkyl;

R7a and R7b are each independently: i) hydrogen; or ii) C1-C4 linear, branched, or cyclic alkyl; or iii) R7a and R7b are taken together to form a ring having from 3 to 7 ring atoms; R3 is hydrogen, methyl, or ethyl;

L is a linking unit having the formula: —[C(R8aR8b)]ₙ—

R8a and R8b are each independently hydrogen, methyl, or ethyl;

the index n is from 1 to 3; and

R9 is hydrogen or methyl; or a pharmaceutically acceptable salt thereof, provided R and R1 are not both hydrogen.

Other suitable embodiments of PHIs are selected from the compounds disclosed in U.S. Pat. No. 7,608,621, which are represented by formula XIII:

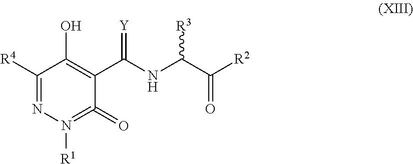

R1 is selected from the group consisting of hydrogen, —NR5R6, C1-C10alkyl, C2-C10alkenyl, C2-C10 alkynyl, C3-C8 cycloalkyl, C1-C10 alkyl-C3-C8 cycloalkyl, C5-C8 cycloalkenyl, C1-C10 alkyl-C5-C8 cycloalkenyl, C3-C8 heterocycloalkyl, C1-C10 alkyl-C3-C8 heterocycloalkyl, aryl, C1-C10 alkyl-aryl, heteroaryl and C1-C10 alkyl-heteroaryl;

R4 is selected from the group consisting of hydrogen, COOR9, CONR7R8, —NR5R6, C1-C10 alkyl, C2-C10 alkenyl, C2-C10 alkynyl, C3-C8 cycloalkyl, C1-C10 alkyl-C3-C8 cycloalkyl, C5-C8 cycloalkenyl, C1-C10 alkyl-C5-C8 cycloalkenyl, C3-C8 heterocycloalkyl, C1-C10 alkyl-C3-C8 heterocycloalkyl, aryl, C1-C10alkyl-aryl, heteroaryl and C1-C10 alkyl-heteroaryl;

### R2 is —NR7R8 or —OR9;

R3 is H or C1-C4alkyl;

R5 and R6 are each independently selected from the group consisting of hydrogen, C1-C10 alkyl, C3-C8 cycloalkyl, C1-C10 alkyl-C3-C8 cycloalkyl, C3-C8 heterocycloalkyl, C1-C10 alkyl-C3-C8 heterocycloalkyl, aryl, C1-C10 alkyl-aryl, heteroaryl, C1-C10 alkyl-heteroaryl, —CO(C1-C4 alkyl), —CO(C3-C6 cycloalkyl), —CO(C3-C6 heterocycloalkyl), —CO(aryl), —CO(heteroaryl), and —SO2(C1-C4 alkyl); or R5 and R6 taken together with the nitrogen to which they are attached form a 5- or 6- or 7-membered saturated ring optionally containing one other heteroatom selected from the group consisting of oxygen, nitrogen and sulphur;

R7 and R8 are each independently selected from the group consisting of hydrogen, C1-C10 alkyl, C2-C10 alkenyl, C2-C10 alkynyl, C3-C8 cycloalkyl, C3-C8 heterocycloalkyl, aryl and heteroaryl;

R9 is H or a cation, or C1-C10alkyl which is unsubstituted or substituted with one or more substituents independently selected from the group consisting of C3-C6 cycloalkyl, heterocycloalkyl, aryl, and heteroaryl;

and wherein any carbon or heteroatom of R1, R2, R3, R4, R5, R6, R7, R8, R9 is unsubstituted or, where possible, is substituted with one or more substituents independently selected from the group consisting of C1-C6 alkyl, aryl, heteroaryl, halogen, —OR10, —NR5R6, cyano, nitro, —C(O)R10, —C(O)OR10, —SR10, —S(O)R10, —S(O)2R10, —NR5R6, —CONR5R6, —N(R5)C(O)R10, —N(R5)C(O)OR10, —OC(O)NR5R6, —N(R5)C(O)NR5R6, —SO2NR5R6, —N(R5)SO2R10, C1-C10 alkenyl, C1-C10 alkynyl, C3-C6 cycloalkyl, C3-C6 heterocycloalkyl, aryl and heteroaryl group; wherein R5 and R6 are the same as defined above and R10 is hydrogen, C1-C10 alkyl, C2-C10 alkenyl, C2-C10 alkynyl, —CO(C₁-C4 alkyl), —CO(aryl), —CO(heteroaryl), —CO(C₃-C6 cycloalkyl), —CO(C₃-C6 heterocycloalkyl), —SO2(C₁-C₄ alkyl), C3-C8 cycloalkyl, C3-C8 heterocycloalkyl, C6-C14 aryl, C1-C10 alkyl-aryl, heteroaryl, or C1-C10 alkyl-heteroaryl;

or a pharmaceutically acceptable salt or solvate thereof.

Other PHIs include without limitation substrate-based inhibitors, such as 3-exomethyleneproline peptide like compounds (Tandon et al. (1998) Bioorg. Med. Chem. Lett. 8:1139-44), derivatives of proline, derivatives of 4(S)hydroxy proline, and derivatives of 4-keto proline. Furthermore, in view of the fact that the activity of PHD polypeptides is iron, 2-oxoglutarate and ascorbic acid dependent (Kivirikko and Pihlajaniemi (1998) T. Adv Enzymol Relat Areas Mol Biol. 72:325-98) and the activity of HIF-α targeting PHDs such as those discussed above is also dependent on these co-factors (Bruick and McKnight (2001) Science 294(5545):1337-40), examples of suitable compounds include cofactor-based inhibitors such as 2-oxoglutarate analogues, ascorbic acid analogues and iron chelators such as desferrioxamine (DFO) and the hypoxia mimetic cobalt chloride (CoCl₂), or other factors that may mimic hypoxia. Also, of interest as compounds suitable for the present invention are prolyl hydroxylase inhibitors, such as deferiprone, 2,2′-dipyridyl, ciclopirox, dimethyloxallyl glycine (DMOG), L-Mimosine (Mim) and 3-Hydroxy-1,2-dimethyl-4(1H)-Pyridone (OH-pyridone). DMOG is a cell permeable, competitive inhibitor of PHDs. It acts to stabilize HIF-α expression at normal oxygen tensions in cultured cells, at concentrations between 0.1 and 1 mM. Other PHD inhibitors encompassed by the present invention include, but are not limited to, oxoglutarates, heterocyclic carboxamides, phenanthrolines, hydroxamates, and heterocyclic carbonyl glycines (including, but not limited to, pyridine carboxamides, quinoline carboxamides, isoquinoline carboxamides, cinnoline carboxamides, beta-carboline carboxamides, including substituted quinoline-2-carboxamides and esters thereof; substituted isoquinoline-3-carboxamides and N-substituted arylsulfonylamino hydroxamic acids (see, e.g., WO 05/007192, WO 03/049686 and WO 03/053997), and the like. Also of interest are compounds described or identified using the methods described in the art, including U.S. Pat. Nos. 6,787,326, and 6,767,705, and 6,436,654; U.S. 2004/0161794, 2004/0152655, 2004/0146964, 2004/0096848, 2004/0087556, 2003/0229108 and 2002/0048794; and WO 04/066949, WO 04/047852, WO 04/043359, WO 04/000328, WO 03/100438, WO 03/085110, WO 03/080566, WO 03/074560, WO 03/049686, WO 03/018014, WO 02/12326, and WO 02/074981 (each herein incorporated by reference).

Other compounds of interest encompassed by the present invention as HIF-α potentiating agents include compounds which interact or modulate the HIF-1 pathway. A general report of such compounds and the pathways associated with HIF-1α levels and HIF-1α activity are disclosed in Semenza (2003, Nature Rev. Cancer 721) Ratcliffe et at (2003, Nature Medicine 677) and Wouters et al. (2004, Drug Resistance Updates 25) (each of which is incorporated herein by reference in its entirety). Illustrative such compounds include without limitation rapamycin (see, e.g., Abraham (2004) Current Topics in Microbiology and Immunology 279:299-319; Arsham et al. (2003) J. Biol Chem. 278(32), 29655-29660), curcumin (see, e.g., Sukhatme, V P. WO 03/094904), fibrostatin (see, e.g., Ishimaru et al. (1988) J Antibiotics, 41(11):1668-74), mimosine (see, e.g., Warnecke et al. (2003) FASEB J. 17(9):1186-1188; Park, et al., WO03/018014; Clement et al. (2002) Int J Cancer 100(4):491-498), 3 hydroxy, 1,2 dimethyl 4-pyridone (see, e.g., Weidmann et al., WO 97/41103; Weidmann et al., EP/650961; Iyer et al. (1998) Exp. Lung Res. 24(1):119-32), camptothecin (see, e.g., Rapisarda et al. (2002) Cancer Res. 62(15):4316-4324), resveratrol (see, e.g., Cao et al., (2004) Clin Cancer Res. 10(15):5253-63), Flavonoids (see, e.g., Hasebe et al. (2003) Biol Pharm Bulletin 26(10):1379-1383; Fan et al. (2003) Eur J Pharm. 481(1):33-40); Majamaa et al. (1984) Eur J Biochem 138:239-245; and Majamaa et al. (1985) Biochem J. 229:127-133; Kivirikko and Myllyharju (1998) Matrix Biol 16:357-368; Bickel et al. (1998) Hepaology 28:404-411; Friedman et al. (2000) Proc Natl Acad Sci USA 97:4736-4741; Franklin (1991) Biochem Soc Trans 19):812-815; and Franklin et al (2001) Biochem J 353:333-338; and the like (each of which is incorporated herein by reference in its entirety). In some embodiments, the HIF-α potentiating agents include the following compounds or derivatives or analogs thereof: Quercetin, 2,4-Diethylpyridine dicarboxylate (2,4-DPD), Dimethyloxaloylglycine (DMOG), 2-(oxalyl-amino)-propionic acid, N-oxalyl glycine (NOG), [2,2′] Bipyridinyl, Dihydroxy benzoic acid, Pyridine 2,4-dicarboxylic acid, 4-Hydroxy-isoquinoline-3-carbonyl glycine, and 8-Nitro-7-oxo-4a,7,8,10b-tetrahydro-[, 10]phenanthroline-3-carboxylic acid.

In addition, many growth factors and cytokines are known to stabilize HIF-α under normoxic conditions, including insulin, insulin-like growth factor, epidermal growth factor, interleukin-1β (Zelzer et a. (1998) EMBO J 17:5085-94; Feldser et al. (1999) Cancer Res 59:3915-8); Richard et al. (2000)J Biol Chem 275:26765-71; Gorlach et al. (2001) Circ Res 89:47-54; Haddad et al. (2001) FEBS Lett 505:269-74; Stiehl et al. (2002) FEBS Lett 512:15-62; Thornton et al. (2000) Biochem J 350 Pt 1, 307; Hellwig-Burgel et al. (1999) Blood 94:1561; Sandau et al. (2001) Blood 97:1009; Zhou et al. (2003) Am J Physiol Cell Physiol 284:C439; Zhou et al. (2003) Mol Biol Cell 14:2216; Kasuno et al. (2004) J Biol Chem 279:2550) (each of which is incorporated herein by reference in its entirety). Similarly, NO and other certain reactive oxygen species are reported to stabilize HIF-1α under normoxia (Brune & Zhou (2003) Curr Med Chem 10(10):845-55; Palmer et al. (2000) Mol Pharmacol 58:1197-203 (each of which is incorporated herein by reference in its entirety). Suitably, such compounds could be utilized as potential lead compounds, to develop additional HIF-α potentiating agents.

Representative compounds and generic structures for deriving other suitable compounds include those described in the following:

Preparation of 3-hydroxypyridine-2-carboxamides for treatment of fibrotic disease. Weidmann, Klaus; Baringhaus, Karl-heinz; Tschank, Georg; Bickel, Martin. (Hoechst A.-G., Germany). EP 900202 A1 (which is incorporated herein by reference in its entirety).

Use of hypoxia-inducible factor-α (HIF-α) stabilizers for enhancing erythropoiesis. Klaus, Stephen J.; Molineaux, Christopher J.; Neff, Thomas B.; Guenzler-Pukall, Volkmar, Lansetmo Parobok, Ingrid; Seeley, Todd W.; Stephenson, Robert C. (Fibrogen, Inc., USA). WO 2004108121 A1 (which is incorporated herein by reference in its entirety).

Preparation of substituted 3-hydroxyquinoline-2-carboxamides as prolyl-4-hydroxylase inhibitors. Weidmann, Klaus; Baringhaus, Karl-Heinz; Tschank, Georg; Bickel, Martin. (Hoechst A.-G., Germany; Fibrogen Inc.). EP 765871 A1 (which is incorporated herein by reference in its entirety).

Pyridinecarboxamides and related compounds for treating fibrotic disease. Weidmann, Klaus; Baringhaus, Karl-Heinz; Tschank, Georg; Bickel, Martin. (Hoechst A.-G., Germany). EP 673929 A1 (which is incorporated herein by reference in its entirety).

Novel inhibitors of prolyl 4-hydroxylase. 5. The intriguing structure-activity relationships seen with 2,2′-bipyridine and its 5,5′-dicarboxylic acid derivatives. Hales, Neil J.; Beattie, John F. Infect Res. Dep., Zeneca Pharm., Macclesfield/Cheshire, UK. Journal of Medicinal Chemistry (1993), 36(24), 3853-8 (which is incorporated herein by reference in its entirety).

Beneficial effects of inhibitors of prolyl 4-hydroxylase in carbon tetrachloride-induced fibrosis of the liver in rats. Bickel, M.; Baader, E.; Brocks, D. G.; Engelbart, K.; Guenzler, V.; Schmidts, H. L.; Vogel, G. H. Hoechst A.-G., Frankfurt, Germany. Journal of Hepatology (1991), 13(Suppl. 3), S26-S34 (which is incorporated herein by reference in its entirety).

Inhibition of prolyl hydroxylase activity and collagen biosynthesis by fibrostatin C, a novel inhibitor produced by Streptomyces catenulae subsp. griseospora No. 23924. Ishimaru, Takenori; Kanamaru, Tsunco; Takahashi, Toshiyuki; Okazaki, Hisayoshi. Cent. Res. Div., Takeda Chem. Ind., Ltd., Osaka, Japan. Journal of Antibiotics (1988), 41(11), 1668-74 (which is incorporated herein by reference in its entirety).

MBP039-06 as proline hydroxylase inhibitor and its manufacture with Phaeosphaeria. Furui, Megumi; Takashima, Junko; Sudo, Keiko; Chiba, Noriko; Mikawa, Takashi. (Mitsubishi Chemical Industries Co., Ltd., Japan). JP 05239023 A2 (which is incorporated herein by reference in its entirety).

The absolute configuration of P-1894B, A potent prolyl hydroxylase inhibitor. Ohta, Kazuhiko; Mizuta, Eiji; Okazaki, Hisayoshi; Kishi, Toyokazu. Cent. Res. Div., Takeda Chem. Ind., Ltd., Osaka, Japan. Chemical & Pharmaceutical Bulletin (1984), 32(11), 4350-9 (which is incorporated herein by reference in its entirety).

Preparation of Novel Curcumin/Tetrahydrocurcumin Derivatives for Use in cosmetics, pharmaceuticals and for nutrition. Rieks, Andre; Kaehler, Markus; Kirchner, Ulrike; Wiggenhorn, Kerstin; Kinzer, Mona. (Andre Rieks-Labor fuer Enzymtechnologie G.m.b.h., Germany). WO 04/031122 (which is incorporated herein by reference in its entirety).

Review on pharmacology in lithospermic acid B. Peng, Zonggen; Chen, Hongshan. Department of Virology, Institute of Medicinal Biotechnology, Chinese Academy of Medical Sciences and Peking Union Medical College, Beijing, Peop. Rep. China. Zhongguo Yaoxue Zazhi (Beijing, China) (2003), 38(10), 744-747 (which is incorporated herein by reference in its entirety).

Proline hydroxylase-inhibiting tetracyclines and their manufacture with Streptomyces species. Furui, Megumi; Takashima, Junko; Sudo, Keiko; Chiba, Noriko; Sashita, Reiko. (Mitsubishi Chemical Industries Co., Ltd., Japan). JP 06339395 A2 (which is incorporated herein by reference in its entirety).

A novel proline hydroxylase inhibitor MBP049-13 and its manufacture with Ophiobolus. Furui, Megumi; Takashima, Junko; Mikawa, Takashi; Yoshikawa, Nobuji; Ogishi, Haruyuki. (Mitsubishi Kasei K. K., Japan). JP 04074163 A2 (each herein incorporated by reference).

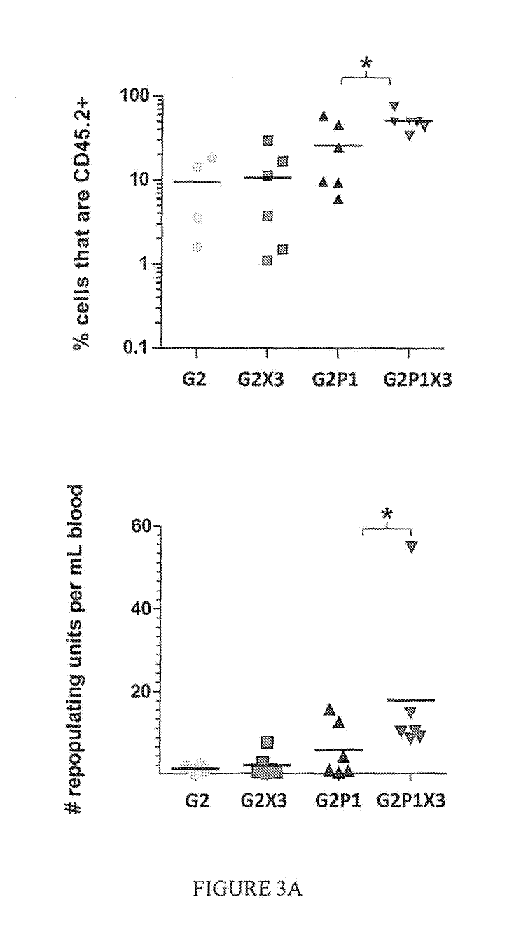

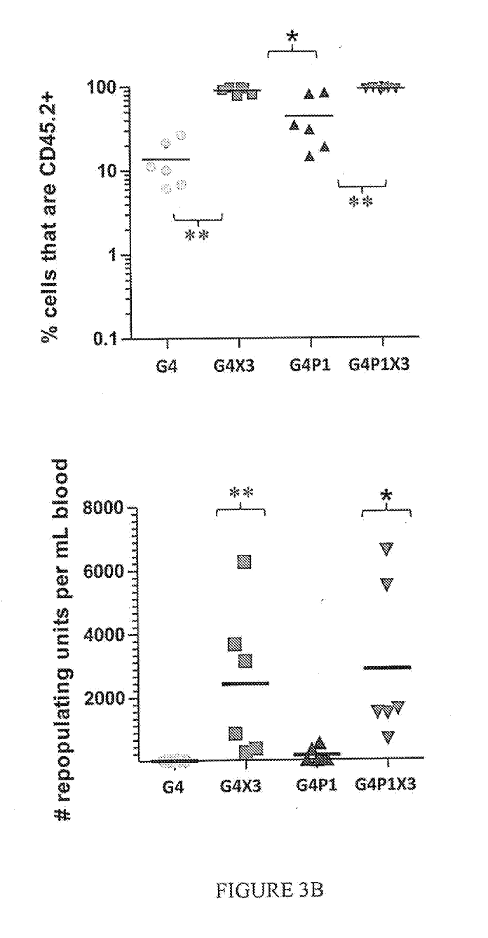

HIF-α contains an oxygen dependent degradation domain (ODDD), which has both an N-terminal portion (NODDD) and a C-terminal portion (CODDD). Hydroxylation at any of the prolyl residues in the ODDD targets the HIF-α subunit to the vHL protein for degradation; therefore, blocking the interaction of vHL with HIF-α leads to build-up of HIF-α. Also, peptides encoding the HIF-α NODDD or CODDD (see, e.g., FIG. 3 of U.S. 2006/0216295, which is incorporated by reference herein in its entirety) are capable of up-regulating HIF-regulated transcripts in vitro (William et al. (2002) Proc Natl Acad Sci. USA 99(16):10423-10428) either by saturating the PHD enzymes or vHL binding, indicating that peptide therapy may also be efficacious.

An alternative strategy is to increase HIF-α mRNA by increasing its transcription. Compounds useful in increasing HIF-α transcription include, for example, o-substituted carbamoyl-phenoxyacetic acids, as disclosed for example by Agani et al. (1998, Mol Pharmacol 54:749-754).

The invention not only encompasses known HIF-α potentiating agents but also HIF-α potentiating agents identified by any suitable screening assay. Accordingly, the present invention extends to methods of screening for modulatory agents that are useful for potentiating HIF-α and, in turn, enhancing a hematopoietic function of a mobilizer of hematopoietic stem cells and/or progenitor cells. In some embodiments, the screening methods comprise (1) contacting a preparation with a test agent, wherein the preparation comprises (i) a polypeptide comprising an amino acid sequence corresponding to at least a fragment of a HIP-α-inhibitory interacting polypeptide selected from a PHD (e.g., a HIF-α PHD) polypeptide, a FIH-1 polypeptide, a vHL polypeptide, or a variant or derivative of any one of these); or (ii) a polynucleotide comprising at least a portion of a genetic sequence (e.g., a transcriptional control sequence) that regulates the expression of a gene selected from a PHD gene, a FIH-1 gene or a vHL gene, wherein the genetic sequence is operably linked to a reporter gene; and (2) detecting a change in the level or functional activity of the polypeptide, or an expression product of the reporter gene, relative to a reference level or functional activity in the absence of the test agent. A detected reduction in the level or activity of polypeptide, or expression product, relative to the reference level or functional activity indicates that the test agent is useful for enhancing a hematopoietic function of a mobilizer of hematopoietic stem cells and/or progenitor cells. Suitably, this is confirmed by analyzing or determining whether the test agent enhances a hematopoietic function of a mobilizer of hematopoietic stem cells and/or progenitor cells. In some embodiments, the test agent inhibits the prolyl hydrolase activity of the PHD, as determined by: contacting a preparation comprising (a) the polypeptide that comprises an amino acid sequence corresponding to at least a fragment of a PHD or variant or derivative thereof and (b) a substrate of the PHD with the test agent and measuring whether the test agent inhibits hydroxylation of proline residues of the substrate (e.g., proline residues of HIF-α). In other embodiments, the test agent inhibits the activity of FIH-1, as determined by: contacting a preparation comprising (i) the polypeptide that comprises an amino acid sequence corresponding to at least a fragment of a FIH-1 or variant or derivative thereof and (ii) a HIF-1 polypeptide with the test agent and measuring whether the test agent enhances the transcriptional activity of the HIF-1 polypeptide. In still other embodiments, the test agent inhibits the activity of vHL, as determined by: contacting a preparation comprising (A) the polypeptide that comprises an amino acid sequence corresponding to at least a fragment of a vHL or variant or derivative thereof and (B) a HIF-α polypeptide or fragment thereof with the test agent and measuring whether the test agent reduces degradation of the HIF-α polypeptide or fragment thereof. Suitably, in the above embodiments, the test agent may inhibit binding between HIF-α and the polypeptide, as determined by: contacting a preparation comprising the HIF-α and the polypeptide with the test agent and measuring the binding of the HIF-α with the polypeptide. In these embodiments, the test agents may bind to the HIF-α or to the polypeptide and test positive when they reduce or abrogate the binding of the HIF-α with the polypeptide.

Modulators falling within the scope of the present invention include antagonists of the level or functional activity of HIF-α-inhibitory interacting polypeptides (e.g., PHD FIH-1 or vHL), including antagonistic antigen-binding molecules, and inhibitor peptide fragments, antisense molecules, ribozymes, RNAi molecules and co-suppression molecules as well as polysaccharide and lipopolysaccharide inhibitors of HIF-α-inhibitory interacting polypeptide function.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, desirably at least two of the functional chemical groups. The candidate agent often comprises cyclical carbon or heterocyclic structures or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of a HIF-α-inhibitory interacting polypeptides are particularly advantageous. In this regard, small molecules are desirable because such molecules are more readily absorbed after oral administration, have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the invention can be achieved by any suitable method. For example, the method may include contacting a cell expressing a polynucleotide corresponding to a gene that encodes a HIF-α-inhibitory interacting polypeptide with an agent suspected of having the modulatory activity and screening for the modulation of the level or functional activity of the HIF-α-inhibitory interacting polypeptide, or the modulation of the level of a transcript encoded by the polynucleotide, or the modulation of the activity or expression of a downstream cellular target of the polypeptide or of the transcript (hereafter referred to as target molecules). Detecting such modulation can be achieved utilizing techniques including, but not restricted to, ELISA, cell-based ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a HIF-α-inhibitory interacting polypeptide is regulated or expressed may be naturally occurring in the cell which is the subject of testing or it may have been introduced into the host cell for the purpose of testing. In addition, the naturally-occurring or introduced polynucleotide may be constitutively expressed—thereby providing a model useful in screening for agents which down-regulate expression of an encoded product of the sequence wherein the down regulation can be at the nucleic acid or expression product level. Further, to the extent that a polynucleotide is introduced into a cell, that polynucleotide may comprise the entire coding sequence that codes for the HIF-α-inhibitory interacting polypeptide or it may comprise a portion of that coding sequence (e.g., the ligand-binding domain of the HIF-α-inhibitory interacting polypeptide) or a portion that regulates expression of the corresponding gene that encodes the HIF-α-inhibitory interacting polypeptide (e.g., a PHD promoter, a FIH-1 promoter, or a vHL promoter). For example, the promoter that is naturally associated with the polynucleotide may be introduced into the cell that is the subject of testing. In this instance, where only the promoter is utilized, detecting modulation of the promoter activity can be achieved, for example, by operably linking the promoter to a suitable reporter polynucleotide including, but not restricted to, green fluorescent protein (GFP), luciferase, galactosidase and catecholamine acetyl transferase (CAT). Modulation of expression may be determined by measuring the activity associated with the reporter polynucleotide.

These methods provide a mechanism for performing high throughput screening of putative modulatory agents such as proteinaceous or non-proteinaccous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression or activity of a target molecule according to the invention.

In specific embodiments, compounds are screened for hydroxylase activity. Assays for hydroxylase activity are standard in the art. Such assays can directly or indirectly measure hydroxylase activity. For example, an assay can measure hydroxylated residues (e.g., proline, etc.) present in the enzyme substrate, e.g., a target protein, a synthetic peptide mimetic, or a fragment thereof (see, e.g., Palmerini et al. (1985) J Chromatogr 339:285-292.) A reduction in hydroxylated residue (e.g., proline, etc.) in the presence of a compound is indicative of a compound that inhibits hydroxylase activity. Alternatively, assays can measure other products of the hydroxylation reaction (e.g., formation of succinate from 2-oxoglutarate (see, e.g., Cunliffe et al. (1986) Biochem J 240:617-619; and Kaule and Gunzler (1990) Anal Biochem 184:291-297).

Procedures such as those described above can be used to identify compounds that inhibit HIP hydroxylase activity. Target protein used in the assay may include HIFα or a fragment thereof, e.g., HIF(556-575). Enzyme may include, e.g., HIF prolyl hydroxylase (see, e.g., GenBank Accession No. AAG33965, etc.) obtained from any source. Human HIF prolyl hydroxylase is preferred. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIF-α.

In certain aspects, a suitable compound is one that stabilizes HIF-α. Compounds that inhibit HIF prolyl hydroxylase prevent or reduce the hydroxylation of the HIFα subunit of the HIF protein. This lack of hydroxylated proline leads to the stabilization (often referred to as activation) of HIF. Determination of the stabilization of HIF by a compound can be used as an indirect measure of the ability of the compound to inhibit HIF prolyl hydroxylase. The ability of a compound to stabilize or activate HIF-α can be measured, for example, by direct measurement of HIF-1α in a sample, indirect measurement of HIF-1α, e.g., by measuring a decrease in HIF-1α associated with the vHL protein (see, e.g., International Publication No. WO 2000/69908), or activation of HIF responsive target genes or reporter constructs (see, e.g., U.S. Pat. No. 5,942,434). Measuring and comparing levels of HIF and/or HIP-responsive target proteins in the absence and presence of the compound will identify compounds that stabilize HIF-1α and/or activate HIF. Suitable compounds for use in the present methods may be identified and characterized using the assay described in International Publication No. WO 2005/118836, or in Example 10 of International Publication No. WO 2003/049686, both of which are incorporated herein by reference in their entirety. Compounds identifiable by these assays are specifically envisaged for use in the present invention.

In alternative embodiments, test agents are screened using the assays disclosed for example in U.S. 2004/0146964, U.S. 2005/0214894, U.S. 2008/0213404, U.S. 2010/0272726 and U.S. 20110301095, each of which are incorporated by reference herein in their entirety.

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

3.2 Mobilizers of Hematopoietic Stem Cells and/or Progenitor Cells

Several classes of agents have been shown to increase the circulation of progenitor and stem cells by “mobilizing” them from the marrow into the peripheral blood. These include agents that decrease the expression or function of a chemokine (the function being the binding of the chemokine to its receptor and further signaling), particularly CXCL12, as well as those that block or antagonize the chemokine receptor, CXCR4.

Accordingly, in some embodiments, the mobilization agent may be an agent that decreases the expression or function of a chemokine, more particularly, CXCL12, also known as SDF-1. The human amino acid sequence of SDF-1 corresponds to GenBank accession number NP_{—}000600. The alpha isoform has GenBank accession number NP_{—}954637. The beta isoform has GenBank accession number NP_{—}000600. The gamma isoform has GenBank accession number NP_{—}001029058.

Alternatively, the mobilization agent may be an agent that blocks or antagonizes a chemokine receptor, in particular, CXCR4. The human amino acid sequence of CXCR4 corresponds to GenBank accession number CAA12166.

Chemokines are a superfamily of chemoattractant proteins. Chemokines regulate a variety of biological responses and they promote the recruitment of multiple lineages of leukocytes and lymphocytes to a body organ tissue. Chemokines may be classified into two families according to the relative position of the first two cysteine residues in the protein. In one family, the first two cysteines are separated by one amino acid residue, the CXC chemokines, and in the other family the first two cysteines are adjacent, the CC chemokines. Two minor subgroups contain only one of the two cysteines (C) or have three amino acids between the cysteines (CX3C). In humans, the genes of the CXC chemokines are clustered on chromosome 4 (with the exception of SDF-1 gene, which has been localized to chromosome 10) and those of the CC chemokines on chromosome 17.

The molecular targets for chemokines are cell surface receptors. One such receptor is CXC chemokine receptor 4 (CXCR4), which is a 7 transmembrane protein, coupled to 01 and was previously called LESTR (Loetscher, M., Geiser, T., O'Reilly, T., Zwahlen, R., Baggionlini, M., and Moser, B., (1994) J. Biol. Chem, 269, 232-237), HUMSTR (Federsppiel, B., Duncan, A. M. V., Delaney, A., Schappert, K., Clark-Lewis, I., and Jirik, F. R. (1993) Genomics 16, 707-712) and Fusin (Feng, Y., Broeder, C. C., Kennedy, P. E., and Berger, E. A. (1996) HIV-1 entry cofactor: Functional cDNA cloning of a seven-transmembrane G protein-coupled receptor, Science 272, 872-877). CXCR4 is widely expressed on cells of hematopoietic origin, and is a major co-receptor with CD4 for human immunodeficiency virus 1 (HIV-1) (Feng, Y., Broeder, C. C., Kennedy, P. E., and Berger, E. A. (1996) HIV-1 entry cofactor: Functional cDNA cloning of a seven-transmembrane G protein-coupled receptor, Science 272, 872-877).

Chemokines are thought to mediate their effect by binding to seven transmembrane G protein-coupled receptors, and to attract leukocyte subsets to sites of inflammation (Baglionini et al. (1998) Nature 392: 565-568). Many of the chemokines have been shown to be constitutively expressed in lymphoid tissues, indicating that they may have a homeostatic function in regulating lymphocyte trafficking between and within lymphoid organs (Kim and Broxmeyer (1999) J. Leuk. Biol. 56: 6-15).

Stromal cell derived factor one (SDF-1), also known as CXCL12, is a member of the CXC family of chemokines that has been found to be constitutively secreted from the bone marrow stroma (Tashiro, (1993) Science 261, 600-602). The human and mouse SDF-1 predicted protein sequences are approximately 92% identical. Stromal cell derived factor-1α (SDF-1α) and stromal cell derived factor-1β (SDF-1β) are closely related (together referred to herein as SDF-1). The native amino acid sequences of SDF-1α and SDF-1β are known, as are the genomic sequences encoding these proteins (see U.S. Pat. No. 5,563,048 issued 8 Oct. 1996, and U.S. Pat. No. 5,756,084 issued 26 May 1998). Identification of genomic clones has shown that the alpha and beta isoforms are a consequence of alternative splicing of a single gene. The alpha form is derived from exons 1-3 while the beta form contains an additional sequence from exon 4. The entire human gene is approximately 10 kb. SDF-1 was initially characterized as a pre-B cell-stimulating factor and as a highly efficient chemotactic factor for B cells and monocytes (Bleul et al. (1996) J. Exp. Med. 184:1101-1110).

Biological effects of SDF-1 may be mediated by the chemokine receptor CXCR4 (also known as fusin or LESTR), which is expressed on mononuclear leukocytes including hematopoietic stem cells. SDF-1 is thought to be the natural ligand for CXCR4, and CXCR4 is thought to be the natural receptor for SDF-1 (Nagasawza et al. (1997) Proc. Natl. Acad. Sci. USA 93:726-732). Genetic elimination of SDF-1 is associated with perinatal lethality, including abnormalities in cardiac development, B-cell lymphopoiesis, and bone marrow myelopoiesis (Nagasawa et al. (1996) Nature 382:635-637). SDF-1 is functionally distinct from other chemokines in that it is reported to have a fundamental role in the trafficking, export and homing of bone marrow progenitor cells (Aiuti, A., et al. (1996) J. Exp. Med. 185, 111-120 and Nagasawa, T., et al. (1996) Nature 382, 635-638). SDF-1 is also structurally distinct in that it has only about 22% amino acid sequence identity with other CXC chemokines.

Agents that decrease the expression of CXCL12 or that block or antagonize CXCR4 may be selected from small organic molecules, polypeptides, nucleic acids and carbohydrates. In more particular embodiments, the polypeptides that decrease the expression of CXCL12 may be selected from the group consisting of a cytokine, a colony stimulating factor, a protease or a chemokine other than CXCL12. The cytokine may be selected from the group consisting of interleukin-1 (IL-1), interleukin-3 (IL-3), interleukin-6 (IL-6), interleukin-11 (IL-11), interleukin-7 (IL-7) and interleukin-12 (IL12). The protease may be selected from the group consisting of a metalloproteinase (like MMP2 or MMP9) a serine protease, (like cathepsin G, or elastase) a cysteine protease (like cathepsin K) and a dipeptidyl peptidase-1 (DDP-1 OR CD26). The chemokine other than CXCL12 may be selected from the group consisting of IL-8, MIP-1α and Groβ. The colony stimulating factor may be selected from the group consisting of granulocyte colony stimulating factor (G-CSF), granulocyte-macrophage colony stimulating factor (GM-CSF), macrophage colony stimulating factor (M-CSF), stem cell factor, FLT-3 ligand or a combination thereof. The nucleic acid may be a DNA or an RNA molecule. The nucleic acid may be a small interfering RNA (siRNA) molecule or an antisense molecule specific for CXCL12 or CXCR4. The carbohydrate may be a sulfated carbohydrate selected from the group consisting of Fucoidan and sulfated dextran.

Suitably, the mobilizer(s) is(are) are selected from colony-stimulating factors such as G-CSF and GM-CSF, erythropoietin (which is now commonly used among cancer patients undergoing chemotherapy to maintain hemoglobin in the near normal range, also has some ability to mobilize CD34⁺ cells), stem cell factor (SCF), polysaccharides such as zymosan, chemokines such as IL-8 and Gro-β, growth factors such as vascular endothelial growth factor (VEGF), and CXCR4 antagonists.

In some embodiments, the mobilizer or at least one of the mobilizers used in combination with a HIF-α potentiating agent is G-CSF or GM-CSF, or their variants, derivatives or analogs. The nucleic acid sequence and encoded amino acid sequence of G-CSF, as well as chemically synthesized polypeptides sharing its biochemical and immunological properties, have been previously disclosed (U.S. Pat. Nos. 6,379,661; 6,004,548; 6,830,705; 5,676,941, 6,027,720; 5,994,518; 5,795,968; 5,214,132; 5,218,092; 6,261,550; 4,810,643; 4,810,321, each of which is incorporated herein by reference in its entirety). Also encompassed are analogs of G-CSF molecules which retain their three-dimensional structures and hybrid molecules maintaining their biological and structural integrity, as described for example by Osslund (U.S. Pat. No. 6,261,550, incorporated herein by reference). Examples of functional G-CSF variants include any proteins, peptides or fragments thereof that are at least 70, 75, 80, 85, 90 or 95% sequence identity or similarity to full-length human G-CSF amino acid sequence or its nucleotide sequence. Modifications of G-CSF to improve functionality or resident serum clearance include but are not limited to polyethyleneglycol and polyethyleneglycol derivatives thereof, glycosylated forms (Lenogastrim™) (WO 2000/44785, incorporated herein by reference), norleucine analogs (U.S. Pat. No. 5,599,690, incorporated herein by reference), addition of amino acids at either terminus to improve folding, stability or targeting, and fusion proteins, such as G-CSF and albumin fusion protein (Albugranin™) (U.S. Pat. No. 6,261,250, incorporated herein by reference). An increase in biological or functional activity over the native peptide may reduce the amount of dose and/or the time period required for treatment. Any chemical or biological entity that functions similar to G-CSF can also be employed. G-CSF, or the drug name Filgrastim, is currently being sold as Neupogen® and its polyethylene glycol modified or pegylated form, with the drug name Pegfilgrastim, sold as Neulasta™.

The coding sequence and amino acid sequence of GM-CSF are known as well as various methods employed to produce recombinant proteins (U.S. Pat. No. 5,641,663, incorporated herein by reference). Examples of functional GM-CSF variants include any proteins, peptides or fragments thereof that are at least 70, 75, 80, 85, 90 or 95% sequence identity or similarity to full-length human GM-CSF amino acid sequence or its coding sequence. Modifications of GM-CSF to improve functionality or resident serum clearance include but are not limited to polyethyleneglycol and polyethyleneglycol derivatives thereof, glycosylated forms, norleucine analogs, addition of amino acids at either terminus to improve folding, stability or targeting, and fusion proteins. An increase in biological or functional activity over the native peptide may reduce the amount of dose and/or the time period required for treatment. Any chemical or biological entity that functions similar to GM-CSF can also be employed. Examples of GM-CSF, or the drug name Sargramostim, which are currently being sold, include Leukine®, Leucomax.® and Leucotropin®.

In specific embodiments, G-CSF, or a variant, derivative or analog thereof, is used either alone or in combination with another mobilizer of HSPCs for concurrent administration with a HIF-α potentiating agent. In illustrative examples of this type, the HIF-α potentiating agent is a PHI (e.g., a small molecule PHI including ones selected from compounds represented by any one of formulae I-IX supra).

In some embodiments, the mobilizer or at least one of the mobilizers used in combination with a HIF-α potentiating agent is a CXCR4 antagonist. Illustrative CXCR4 antagonists include aromatic-linked polyamine macrocyclic compounds, as described for example in U.S. Pat. No. 5,583,131, reissued as U.S. RE 42,152, which is expressly incorporated herein by reference in its entirety. In one aspect, the CXCR4 antagonist is 1,1′-[1,4-phenylenebis(methylene)]-bis-1,4,8,11-tetra-azacyclotetradecane (AMD3100; Plerixafor, Mozobil®).

In other embodiments, small molecule CXCR4 antagonists may be selected from macrocyclic compounds disclosed in U.S. Pat. Appl. Pub. No. 2012/0301427, which is expressly incorporated herein by reference in its entirety. These compounds comprise a “core” nitrogen atom surrounded by three pendant groups, wherein two of the three pendant groups are suitably benzimidazolyl methyl and tetrahydroquinolinyl, and the third is a pendant group contains an additional nitrogen.

Still other embodiments of small molecule CXCR4 antagonists include compounds disclosed in U.S. Pat. Appl. Pub. No. 2012/0101280, which is expressly incorporated herein by reference in its entirety.

In other embodiments, CXCR4 antagonists are selected from β-hairpin peptidomimetics as disclosed for example in U.S. Pat. Appl. Pub. No. 2012/0283196, which is expressly incorporated herein by reference in its entirety.

In specific embodiments, a CXCR4 antagonist as described in the foregoing patents and patent applications, for example, Plerixafor, is used either alone or in combination with another mobilizer of HSPCs for concurrent administration with a HIF-α potentiating agent. In illustrative examples of this type, the HIF-α potentiating agent is a PHI.

Suitably, at least two different mobilizers are used for concurrent administration with the HIF-α potentiating agent. In specific embodiments, the at least two mobilizers comprise a CXCR4 antagonist and a colony stimulating factor such as G-CSF or GM-CSF, or variants, derivatives or analogs thereof. In illustrative examples of this type, the CXCR4 antagonist is Plerixafor, or similar compounds, and the colony stimulating factor is G-CSF or a variant, derivative or analog thereof. In these embodiments, the HIF-α potentiating agent is suitably a PHI (e.g., a small molecule PHI including, but not limited to, ones selected from compounds according to any one of formulae I-IX).

### 4. Therapeutic and Prophylactic Uses

In accordance with the present invention, it is proposed that HIF-α potentiating agents are useful as actives for enhancing the hematopoietic properties of mobilizers of hematopoietic stem cells and/or progenitor cells. Thus, a HIF-α potentiating agent can be administered to an individual concurrently (e.g., in the same composition or in separate compositions) with at least one mobilizer (“combination treatment”), and optionally with a pharmaceutically acceptable carrier, to stimulate or enhance hematopoiesis including the mobilization of hematopoietic stem cells and/or progenitor cells, including granulocytes/macrophage progenitors and/or megakaryocyte/erythrocyte progenitors, from the bone marrow, and more particularly to increase the number of hematopoietic stem cells, progenitor cells and granulocytes such as neutrophils in a patient, particularly in the peripheral blood.

In one aspect, the present invention thus provides a method for mobilizing hematopoietic stem cells and/or progenitor cells from bone marrow into peripheral blood of a donor subject, the method comprising, administering to the subject a HIF-α potentiating agent in an effective amount to mobilize hematopoietic stem cells and/or progenitor cells from the bone marrow into the peripheral blood of the subject. A suitable donor subject in this embodiment is one that has been, is, or will be administered a mobilizer.

In another aspect, the present invention provides a method for mobilizing hematopoietic stem cells and/or progenitor cells from bone marrow into peripheral blood of a donor subject, the method comprising, consisting or consisting essentially of: administering concurrently to the subject a HIF-α potentiating agent and at least one mobilizer of hematopoietic stem cells and/or progenitor cells in effective amounts to mobilize hematopoietic stem cells and/or progenitor cells from the bone marrow into the peripheral blood of the subject.

The above methods may further comprise collecting or harvesting mobilized hematopoietic stem cells and/or progenitor cells from the subject, and optionally culturing and/or storing the collected or harvested mobilized hematopoietic stem cells and/or progenitor cells, and further optionally transplanting the collected or harvested mobilized hematopoietic stem cells and/or progenitor cells into a recipient subject.

The dosages of HIF-α potentiating agent and the at least one mobilizer to be administered may depend on the subject to be treated inclusive of the age, sex, weight and general health condition thereof. The dosages will also take into consideration the binding affinity or modulatory activity of the HIF-α potentiating agent to its target molecule, the hematopoietic capacity of the mobilizer(s), their bioavailability and their in vivo and pharmacokinetic properties. In this regard, precise amounts of the agents for administration can also depend on the judgment of the practitioner. In determining the effective amount of the agents to be administered in the treatment of an immunocompromised condition, the physician or veterinarian may evaluate the progression of the disease or condition over time. In any event, those of skill in the art may readily determine suitable dosages of the agents of the invention without undue experimentation. The dosage of the active agents administered to a patient should be sufficient to effect a beneficial response in the patient over time such as enhanced hematopoiesis or a reduction in the symptoms associated with an immunocompromised condition, including a reduction in anemia, thrombocytopenia, agranulocytosis and/or neutropenia. The dosages may be administered at suitable intervals to boost hematopoiesis or ameliorating the symptoms of the immunocompromised condition. Such intervals can be ascertained using routine procedures known to persons of skill in the art and can vary depending on the type of active agent employed and its formulation. For example, the interval may be daily, every other day, weekly, fortnightly, monthly, bimonthly, quarterly, half-yearly or yearly.

Dosage amount and interval may be adjusted individually to provide plasma levels of the active agent which are sufficient to maintain HIF-α potentiating agent modulatory effects and hematopoietic function enhancing effects. Usual patient dosages for systemic administration range from 1-2000 mg/day, 10-1000 mg/day, 50-500 mg/day, 100-800 mg/day, commonly from 1-250 mg/day, and typically from 10-150 mg/day. Stated in terms of patient body weight, usual dosages range from 0.02-25 mg/kg/day, 0.5-15 mg/kg/day, 1.0-10 mg/kg/day, 1-5 mg/kg/day, commonly from 0.02-3 mg/kg/day, typically from 0.2-1.5 mg/kg/day. Stated in terms of patient body surface areas, usual dosages range from 0.5-1200 mg/m²/day, 10-800 mg/m²/day, 50-500 mg/m²/day, 75-200 mg/m²/day commonly from 0.5-150 mg/m²/day, typically from 5-100 mg/m²/day. Animal testing of effective doses for treatment of particular disorders will provide further predictive indication of human dosage. Various considerations are described, e.g., in Gilman et al., (eds.) (1990) Goodman and Gilman's: The Pharmacological Bases of Therapeutics, 8th Ed., Pergamon Press, and Remington's Pharmaceutical Sciences, 17th ed. (1990), Mack Publishing Co., Easton, Pa. Methods for administration are discussed therein, e.g., for oral, intravenous, intraperitoneal, or intramuscular administration, transdermal diffusion, and others. Pharmaceutically acceptable carriers will generally include water, saline, buffers, and other compounds described, e.g., in the Merck Index, Merck & Co., Rahway, N.J.

Thus, the HIF-α potentiating agent and the mobilizer(s) may be provided in effective amounts to stimulate or enhance hematopoiesis. This process may involve administering the HIF-α potentiating agent separately, simultaneously or sequentially with the mobilizer(s). In some embodiments, this may be achieved by administering a single composition or pharmacological formulation that includes both types of agent, or by administering two or more separate compositions or formulations at the same time, wherein one composition includes the HIF-α potentiating agent and the other(s), the mobilizer(s). It will be appreciated that if more than one mobilizer is used, the mobilizers may be administered separately, simultaneously or sequentially.

In other embodiments, the treatment with the HIF-α potentiating agent may precede or follow the treatment with the mobilizer(s) by intervals ranging from minutes to days. In embodiments where the HIF-α potentiating agent is applied separately to the mobilizer(s), one would generally ensure that a significant period of time did not expire between the time of each delivery, such that the HIF-α potentiating agent would still be able to exert an advantageously combined effect on hematopoiesis with the mobilizer(s), in particular, to maintain or enhance a subject's capacity to mobilize hematopoietic stem cells and/or progenitor cells including an increase in the number of granulocytes such as neutrophils. In such instances, it is contemplated that one would administer both modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations.

It is conceivable that more than one administration of either the HIF-α potentiating agent or mobilizer(s) will be desired. Various combinations may be employed, where the HIF-α potentiating agent is “A” and the mobilizer(s) is(are) “B”, as exemplified below:

A/B/A B/A/B B/B/A/ A/A/B/ B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B. Alternately, A/B/A B/A/B/A/B, etc.; B/B/A/ A/B/B/A/A/B/B, etc.; B/B/B/A B/B/B/A/B/B/B, etc. A/A/B/B A/B/A/B/ A, etc. B/B/A B/B/A/A B/B/A/A, etc. B/A BB/A/A/B B/B/A/ A/A, etc.; and A/A/A/B/A/B/A/A /A/B/A/B, etc. Other combinations are contemplated. Again, both agents are delivered to a subject's immune system in a combined amount effective to enhance hematopoiesis as compared to the administration of the same amount of mobilizer alone.

The HIF-α potentiating agent and the mobilizer(s) may be administered directly to a subject or it may be desirable to conjugate one or both to carrier proteins such as ovalbumin or serum albumin prior to administration. While it is possible for the active agent to be administered alone, it is generally desirable to present it as a pharmaceutical composition. Such compositions typically comprise at least one active agent or ingredient, together with one or more acceptable carriers thereof. Each carrier should be both pharmaceutically and physiologically acceptable in the sense of being compatible with the other ingredients and not injurious to the patient. Compositions include those suitable for oral, rectal, nasal, topical, or parenteral (including subcutaneous, intramuscular, intravenous and intradermal) administration. The compositions may conveniently be presented in unit dosage form and may be prepared by many methods well known in the art of pharmacy. See, e.g., Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds.) (1993) Pharmaceutical Dosage Forms, Parenteral Medications Dekker, N.Y.; Lieberman, et al. (eds.) (1990) Pharmaceutical Dosage Forms: Tablets Dekker, N.Y.; and Lieberman, et al. (eds.) (1990) Pharmaceutical Dosage Forms: Disperse Systems Dekker, N.Y. The methods of the invention may be combined with or used in association with other therapeutic agents.

The methods and uses of the present invention are useful for mobilizing HSPCs in subjects. The subjects may have an immunocompromised condition or have an increased risk of acquiring an immunocompromised condition, which may result for example from a congenital disorder (e.g., congenital leukopenia), childhood or adult cyclic neutropenia, post-infective neutropenia, and myelodysplastic syndrome or a medical treatment such as induced by treatment with cytoreductive, myeloablative or immunosuppressive therapies (e.g., chemotherapy, radiation therapy and immunosuppressive drugs such as steroid), in particular in relation to the treatment of transplant rejection and the treatment of hyperproliferative cell disorders such as cancer and autoimmune disease. Alternatively, the subjects are individuals who may serve as allogeneic, syngeneic or xenogeneic donors of HSPCs and the treatment is used to mobilize and collect HSPCs for subsequent delivery to a recipient who has an immunocompromised condition or has an increased risk of acquiring an immunocompromised condition. In addition, the treatment can be used for patients or donors who are “difficult to mobilize” because, for example, they are not sensitive to growth factors. The treatment can further be used to cause tolerance of a recipient for organ transplantation.

The treatment can also be used in cancer therapy methods and in methods for inhibiting, ameliorating, or ablation of cancer cells and/or tumors. For example, like normal HSPCs, their malignant counterparts, leukemia initiating cells (LICs) reside in their bone marrow (BM) niches that provide the structural and physiological conditions supporting their survival and growth. LICs are resistant to traditional cancer therapy (e.g., cytoreductive or myeloablative therapy) by interacting with their BM microenvironment, which can be the culprits of leukemia relapses after a period of remission induced by a cancer therapy (e.g., cytoreductive or myeloablative therapy). Detachment of LICs from their niche by inducing mobilization of LICs (e.g., by administering the treatment) can be a used in combination with traditional cancer therapies (e.g., cytoreductive or myeloablative therapies) to provide more effective or improved cancer therapy methods and methods for inhibiting, ameliorating, or ablation of cancer cells and/or tumors.

The treatment can additionally be used for gene therapy. Because pluripotent hematopoietic stem cells are self-renewing, and give rise to cell progenitors as well as mature blood cells, the stem cells are an appropriate target for gene therapy. After mobilization, HSPCs can be collected. The HSPCs can be modified to deliver gene products upon reintroduction to the individual. After modification, the cells are reinfused into the affected individual.

In some embodiments, the treatment is administered to a patient to stimulate or enhance mobilization of HSPCs from the bone marrow into the peripheral blood and the mobilized HSPCs are then collected or harvested from the patient. Blood containing mobilized HSPC may be collected from the donor by means well known in the art. In a typical protocol, the mobilized cells are collected from the donor by, for example, apheresis and then stored/cultured/expanded/fractionated as desired. In order to ensure capture of a repopulating quantity of cells, it is generally desirable to collect the donor's blood when the levels of mobilized HSPCs peak. In order to optimize the number of HSPCs harvested from mobilized blood, the levels HSPCs can be monitored by methods well known to those of skill in the art, and collection timed to coincide with HSPC peaks.

If desired, the donor cells can be enriched ex vivo by treating them with factors that stimulate the TNFα and GM-CSF receptors. Alternatively, or in addition, factors that stimulate FLT3 and the G-CSF receptor, such as FL and G-CSF, may be used. More particularly, hematopoietic tissues such as bone marrow and blood can be harvested from a donor by methods well known to those skilled in the art, and treated with TNFα, GM-CSF, FL, SCF, IL-7, IL-12, and G-CSF, either singularly or in combination, to enrich selectively for HSPCs.

The cells harvested from the donor are typically cultured ex vivo for several days in medium supplemented with TNFα, GM-CSF, FL, SCF, IL-7, IL-12, and G-CSF, either singularly or in combination. The concentration of GM-CSF administered would typically be in the range of 1,000 U/mL. In an alternative embodiment, TNFα may be administered, typically at a concentration of 200 U/mL. Appropriate concentrations of G-CSF, SCF, IL-7, IL-12, and FL can be readily determined by those of skill in the art, as by titration experiments or by reference to the working examples provided herein.

In some applications, it may be desirable to treat the cultured cells to remove graft versus host disease (GVHD) causing cells, using routine methods known in the art, as for example discussed below. The enriched HSPCs may then be selectively collected from the culture using techniques known to those of skill in the art, as for example discussed below.

In order to ensure enrichment of HSPCs to a repopulating quantity, it is generally desirable to collect the cultured cells when the levels of HSPCs peak. As with in vivo mobilization, ex vivo enrichment of cultured hematopoietic cells produces peak levels of HSPCs on different days depending on the cytokine administration protocol used. In order to optimize the number of HSPCs collected from cultured cells, the levels of HSPCs can be monitored by methods well known to those of skill in the art, and collection timed to coincide with HSPCs peaks.

Following collection, the HSPCs can be resuspended, stored, expanded and/or fractionated and administered to a recipient. The recipient may be the original donor and thus the administration of HSPCs is for an autologous stem cell transplantation. Alternatively, the recipient is not the donor and the administration of HSPCs is for an allogeneic syngeneic or xenogeneic stem cell transplantation. In some embodiments, the allogenic or xenogenic stem cells are transplanted after the recipient undergoes a non-myeloablative conditioning regimen (a “mini-allogenic” or “mini-xenogenic” stem cell transplantation).

Once the HSPCs have been mobilized into a subject's peripheral blood or enriched in the cultured cells, they may be used as donor cells in the form of total white blood cells or peripheral blood mononuclear cells, or selectively enriched by various methods which utilize specific antibodies which suitably bind specific markers to select those cells possessing or lacking various markers. These techniques may include, for example, flow cytometry using a fluorescence activated cell sorter (FACS) and specific fluorochromes, biotin-avidin or biotin-streptavidin separations using biotin conjugated to cell surface marker-specific antibodies and avidin or streptavidin bound to a solid support such as affinity column matrix or plastic surfaces, magnetic separations using antibody-coated magnetic beads, destructive separations such as antibody and complement or antibody bound to cytotoxins or radioactive isotopes.

If the mobilized blood is used for an autologous transplant, the peripheral blood mononuclear cells (PBMC) may be re-infused into the patient without modifications, with the exception that in the case of a cancer patient, the cell preparation is generally first purged of tumor cells. In contrast, if the mobilized blood is transferred into an allogeneic or xenogeneic recipient, the PBMC may first be depleted of GHVD-producing cells, leaving the HSPCs enriched in the PBMC population. In that connection, the PBMC may be treated with anti-αβTCR and anti-γδTCR antibodies to deplete T cells, anti-CD19 to deplete B cells and anti-CD56 to deplete NK cells. It is important to note that anti-Thy-1 antibodies should not be used to deplete GVHD producing cells, as they would deplete T cells and HSPCs. Therefore, it is important to choose carefully the appropriate markers as targets for selecting the cells of interest and removing undesirable cell types.

Separation via antibodies for specific markers may be by negative or positive selection procedures. In negative separation, antibodies are used which are specific for markers present on undesired cells. Cells bound by an antibody may be removed or lysed and the remaining desired mixture retained. In positive separation, antibodies specific for markers present on the desired cells are used. Cells bound by the antibody are separated and retained. It will be understood that positive and negative separations may be used substantially simultaneously or in a sequential manner.

The most common technique for antibody based separation has been the use of flow cytometry such as by a FACS. Typically, separation by flow cytometry is performed as follows. The suspended mixture of hematopoietic cells are centrifuged and resuspended in media. Antibodies which are conjugated to fluorochrome are added to allow the binding of the antibodies to specific cell surface markers. The cell mixture is then washed by one or more centrifugation and resuspension steps. The mixture is run through a FACS which separates the cells based on different fluorescence characteristics. FACS systems are available in varying levels of performance and ability, including multi-color analysis. The HSPCs can be identified by a characteristic profile of forward and side scatter which is influenced by size and granularity, as well as by positive and/or negative expression of certain cell surface markers.

Other separation techniques besides flow cytometry may provide for faster separations. One such method is biotin-avidin based separation by affinity chromatography. Typically, such a technique is performed by incubating the washed bone marrow with biotin-coupled antibodies to specific markers followed by passage through an avidin column. Biotin-antibody-cell complexes bind to the column via the biotin-avidin interaction, while other cells pass through the column. Finally, the column-bound cells may be released by perturbation or other methods. The specificity of the biotin-avidin system is well suited for rapid positive separation.

Flow cytometry and biotin-avidin techniques provide highly specific means of cell separation. If desired, a separation may be initiated by less specific techniques which, however, can remove a large proportion of non-HSPC from the hematopoietic cell source. It is generally desirable to lyse red blood cells from mobilized blood before use. For example, magnetic bead separations may be used to initially remove lineage committed, differentiated hematopoietic cell populations, including T-cells, B-cells, natural killer (NK) cells, and macrophages (MAC), as well as minor cell populations including megakaryocytes, mast cells, eosinophils, and basophils. Desirably, at least about 70% and usually at least about 80% of the total hematopoietic cells present can be removed.

An exemplary initial separation technique is density-gradient separation. Here, the mobilized blood is centrifuged and the supernatant removed. The cells are resuspended in, for example, RPMI 1640 medium (Gibco) with 10% HSA and placed in a density gradient prepared with, for example, Ficoll or Percoll or Eurocollins media. The separation may then be performed by centrifugation or may be performed automatically with, for example, a Cobel & Cell Separator '2991 (Cobev, Lakewood, Colo.). Additional separation procedures may be desirable depending on the source of the hematopoietic cell mixture and on its content.

The HSPCs contained in enriched cell cultures or mobilized blood may be used in the form of total mononuclear cells, or partially purified or highly purified cell populations. If these cellular compositions are separate compositions, they are suitably administered simultaneously, but may be administered separately within a relatively close period of time. The mode of administration is generally but not limited to intravenous injection.

Once administered, it is believed that the cells home to various hematopoietic cell sites in the recipient's body, including bone marrow. The number of cells which should be administered is calculated for a specific species of recipient. For example, in rats, the T-cell depleted bone marrow component administered is typically between about 1×10⁷ cells and 5×10⁷ cells per recipient. In mice, the T-cell depleted bone marrow component administered is typically between about 1×10⁶ cells and 5×10⁶ cells per recipient. In humans, the T-cell depleted bone marrow component administered is typically between about 1×10⁸ cells and 3×10⁸ cells per kilogram body weight of recipient. For cross-species engraftment, larger numbers of cells may be required.

In mice, the number of HSPCs administered is suitably between about 100 and 300 HSPCs per recipient. In rats, the number of HSPCs administered is generally between about 600 and 1200 HSPCs per recipient. In humans, the number of HSPCs administered is suitably between about 1×10⁵ and 1×10⁶ HSPC per recipient. The amount of the specific cells used will depend on many factors, including the condition of the recipient's health. In addition, co-administration of cells with various cytokines may further promote engraftment.

In addition to total body irradiation, a recipient may be conditioned by a medical treatment that results in immunosuppression and myeloablation or cytoreduction by the same techniques as are employed in substantially destroying a recipient's immune system, including, for example, irradiation, toxins, antibodies bound to toxins or radioactive isotopes, or some combination of these techniques. However, the level or amount of agents used is substantially smaller when immunosuppressing and cytoreducing than when substantially destroying the immune system. For example, substantially destroying a recipient's remaining immune system often involves lethally irradiating the recipient with 950 rads (R) of total body irradiation (TBI). This level of radiation is fairly constant no matter the species of the recipient Consistent xenogeneic (rat→mouse) chimerism has been achieved with 750 R TBI and consistent allogeneic (mouse) chimerism with 600R TBT. Chimerism was established by PB typing and tolerance confirmed by mixed lymphocyte reactions (MLR) and cytotoxic lymphocyte (CTL) response.

The mobilized blood and enriched cultured cells prepared in accordance with the present invention may be used for establishing both allogeneic chimerism and xenogeneic chimerism. Xenogeneic chimerism may be established when the donor and recipient as recited above are different species. Xenogeneic chimerism between rats and mice, between hamsters and mice, and between chimpanzees and baboons has been established. Xenogeneic chimerism between humans and other primates is also possible. Xenogeneic chimerism between humans and other mammals, such as pig, is equally viable.

It will be appreciated that, though the methods disclosed above involve one recipient and one donor, the present invention encompasses methods in which HSPCs from two donors are engrafted in a single recipient.

In some embodiments, the mobilized cells and enriched cultured cells prepared using inter alia the combination treatment of the present invention are useful in reestablishing a recipient's hematopoietic system by substantially destroying the recipient's immune system or immunosuppressing and myeloablating or cytoreducing the recipient's immune system, and then administering to the recipient syngeneic or autologous cell compositions comprising syngeneic or autologous purified HSPCs which are MHC-identical to the HSPCs of the recipient.

As noted above, the combination treatment of the present invention also finds utility inter alia in the treatment or prophylaxis of immunocompromised conditions, including those resulting from medical treatment that target hematopoietic stem cells, such as treatments that target rapidly dividing cells or that disrupt the cell cycle or cell division and that result in immunosuppression and myeloablation or cytoreduction. The HIF-α potentiating agent and mobilizer(s) of hematopoietic stem cells and/or progenitor cells may be used therapeutically after the medical treatment or may be used prophylactically before the treatment is administered or together with the medical treatment. Accordingly, the present invention contemplates further combination therapies which employ both a medical treatment that induces an immunocompromised condition and concurrent administration of an HIF-α potentiating agent antagonist and at least one mobilizer of hematopoietic stem cells and/or progenitor cells.

It is well known that chemotherapy and radiation therapy target rapidly dividing cells and/or disrupt the cell cycle or cell division. These treatments are offered as part of the treating hyperproliferative cell disorders including cancers and autoimmune diseases, aiming either at slowing their progression or reversing the symptoms of disease by means of a curative treatment. In some embodiments, therefore, the combination treatment is used for treating a hyperproliferative cell disorder, including cancers and autoimmune diseases.

Representative cancers contemplated by the present invention include, but are not limited to, sarcomas, melanomas, adenomas, carcinomas of solid tissue (e.g., breast, ovary, prostate, colon, lung, skin, kidney, bladder, pancreas, head and neck) including squamous cell carcinomas of the mouth, throat, larynx, and lung, hypoxic tumors; hematopoietic cancers; nervous system cancers; benign lesions such as papillomas; leukemias, and lymphomas, illustrative examples of which include carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, chorioc arcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, acute promyelocytic leukemia, chronic myelogenous leukemia, chronic myelomonocytic leukemia, acute lymphoblastic leukemia, chronic lymphocytic leukemia, granulocytic leukemia, hairy cell leukemia, juvenile myelomonocytic leukemia, large granular lymphocytic leukemia, monocytic leukemia, T-cell leukemia, T-cell prolymphocytic leukemia), lymphomas (e.g., ATDS-related lymphoma, Burkitt's lymphoma, follicular lymphoma, mantle cell lymphoma, diffuse large B-cell lymphoma, malignant lymphoma, MALT lymphoma, mycosis fungoides, plasmocytoma, precursor T-cell lymphoma, reticulum cell sarcoma, Thyroid lymphoma, or Hodgkin's disease including nodular sclerosing or mixed-cellularity subtypes), and tumors of the nervous system including glioma, meningoma, medulloblastoma, schwannoma and epidymoma. In specific embodiments, the cancer is leukemia, non-Hodgkin's lymphomas or multiple myeloma.

Non-limiting examples of additionally hematologic disorders contemplated by the present invention include myelodysplastic syndromes (e.g., refractory anemia, refractory cytopenia, chronic myelomonocytic leukemia or unclassifiable myeodlysplastic syndrome), myeloproliferative disorders (e.g., polycythemia vera, essential thrombocytosis and primary or idiopathic myelofibrosis) and aplastic anemia.

Non-limiting examples of autoimmune diseases contemplated by the present invention include Chagas disease, chronic obstructive pulmonary disease, Crohn's disease (one of two types of idiopathic inflammatory bowel disease “IBD”), dermatomyositis, diabetes mellitus type 1, endometriosis, Goodpasture's syndrome, Graves' disease, Guillain-Bane syndrome (GBS), Hashimoto's disease, hidradenitis suppurativa, Kawasaki disease, IgA nephropathy, idiopathic thrombocytopenic purpura, interstitial cystitis, lupus erythematosus, mixed connective tissue disease, morphea, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, schizophrenia, scleroderma, Sjogren's syndrome, stiff person syndrome, temporal arteritis (also known as “giant cell arteritis”), ulcerative colitis (one of two types of idiopathic inflammatory bowel disease “IBD”), vasculitis, vitiligo, Wegener's granulomatosis, celiac disease, chronic thyroiditis (Hashimoto's thyroiditis), pernicious anemia, autoimmune hepatitis, Behcet's disease, uveitis, atherosclerosis, stroke, anti-phospholipid antibody syndrome, and the like.

Accordingly, in some embodiments, the treatment will additionally employ a chemotherapeutic agent, which is suitable selected from cytostatic agents and cytotoxic agents. Non-limiting examples of cytostatic agents are selected from: (1) microtubule-stabilizing agents such as but not limited to taxanes, paclitaxel, docetaxel, epothilones and laulimalides; (2) kinase inhibitors, illustrative examples of which include Iressa®, Gleevec, Tarceva™, (Erlotinib HCl), BAY-43-9006, inhibitors of the split kinase domain receptor tyrosine kinase subgroup (e.g., PTK787/ZK 222584 and SU11248); (3) receptor kinase targeted antibodies, which include, but are not limited to, Trastuzumab (Herceptin®), Cetuximab (Erbitux®), Bevacizumab (Avastin™), Rituximab (Ritusan®), Pertuzumab (Omnitarg™); (4) mTOR pathway inhibitors, illustrative examples of which include rapamycin and CC1-778; (5) Apo2L/Trail, anti-angiogenic agents such as but not limited to endostatin, combrestatin, angiostatin, thrombospondin and vascular endothelial growth inhibitor (VEGI); (6) antineoplastic immunotherapy vaccines, representative examples of which include activated T-cells, non-specific immune boosting agents (i.e., interferons, interleukins); (7) antibiotic cytotoxic agents such as but not limited to doxorubicin, bleomycin, dactinomycin, daunorubicin, epirubicin, mitomycin and mitozantrone; (8) alkylating agents, illustrative examples of which include Melphalan, Carmustine, Lomustine, Cyclophosphamide, Ifosfamide, Chlorambucil, Fotemustine, Busulfan, Temozolomide and Thiotepa; (9) hormonal antineoplastic agents, non-limiting examples of which include Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (10) gonadal hormones such as but not limited to Cyproterone acetate and Medoxyprogesterone acetate; (11) antimetabolites, illustrative examples of which include Cytarabine, Fluorouracil, Gemcitabine, Topotecan, Hydroxyurea, Thioguanine, Methotrexate, Colaspase, Raltitrexed and Capicitabine; (12) anabolic agents, such as but not limited to, Nandrolone; (13) adrenal steroid hormones, illustrative examples of which include Methylprednisolone acetate, Dexamethasone, Hydrocortisone, Prednisolone and Prednisone; (14) neoplastic agents such as but not limited to Irinotecan, Carboplatin, Cisplatin, Oxaliplatin, Etoposide and Dacarbazine; and (15) topoisomerase inhibitors, illustrative examples of which include topotecan and irinotecan.

Illustrative cytotoxic agents can be selected from sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofuiven, dexifosfamide, cis-aminedichloro(2-methyl-pyridine)platinum, benzylguanine, glufosfamide, GPX 100, (trans, trans, trans)-bis-mu-(hexane-1,6-diamine)-mu-[diamine-platinum(II)]bis[diamine(chloro) platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see International Publication WO 00/50032), methoxtrexate, gemcitabine, and mixture thereof.

In some embodiments, the concurrent administration of the HIF-α potentiating agent and the mobilizer(s) is used in combination with radiotherapies, such as but not limited to, conformal external beam radiotherapy (10-100 Grey given as fractions over 4-8 weeks), either single shot or fractionated, high dose rate brachytherapy, permanent interstitial brachytherapy, systemic radio-isotopes (e.g., Strontium 89) or radiaolabeled antibodies or peptides. In illustrative examples of this type, the radiotherapy is administered in combination with a radiosensitizing agent. Illustrative examples of radiosensitizing agents include but are not limited to efaproxiral, etanidazole, fluosol, misonidazole, nimorazole, temoporfin and tirapazamine.

Immunocompromised conditions generally lead to pathogenic infections and thus the present invention also extends to the treatment and/or prophylaxis of infections in individuals suffering from an immunocompromised condition, or to treatment of individuals who are likely to contract such a condition due to treatment known to be associated with the occurrence of an immunocompromised condition. Accordingly, an immunocompromised condition arising from a medical treatment is likely to expose the individual in question to a higher risk of infection. It is possible according to the invention to prophylactically treat an infection in an individual having the immunocompromised condition before or during treatments known to generate such a condition. By prophylactically treating with concurrent administration of the HIF-α potentiating agent and one or more mobilizers (also referred to herein as an “HIF-α potentiating agent/mobilizer combination” or “combination treatment”) before or during a treatment known to generate an immunocompromised condition it is possible to prevent a subsequent infection or to reduce the risk of the individual contracting an infection manifesting from that condition. In some embodiments, therefore, the present invention extends to ancillary combination therapies, which employ both the HIF-α potentiating agent/mobilizer combination and an anti-infective agent that is effective against an infection that develops or that has an increased risk of developing from an immunocompromised condition resulting from a medical treatment as broadly described above.

The anti-infective agent is suitably selected from antimicrobials, which include without limitation compounds that kill or inhibit the growth of microorganisms such as viruses, bacteria, yeast, fungi, protozoa, etc. and thus include antibiotics, amebicides, antifungals, antiprotozoals, antimalarials, antituberculotics and antivirals. Anti-infective agents also include within their scope anthelmintics and nematocides. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin; gemifloxacin; and garenoxacin), tetracyclines, glycylcyclines and oxazolidinones (e.g., chlortetracycline, demeclocycline, doxycycline, lymecycline, methacycline, minocycline, oxytetracycline, tetracycline, tigecycline; linezolide, eperozolid), glycopeptides, aminoglycosides (e.g., amikacin, arbekacin, butirosin, dibekacin, fortimicins, gentamicin, kanamycin, meomycin, netilmicin, ribostamycin, sisomicin, spectinomycin, streptomycin, tobramycin), β-lactams (e.g., imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefbtaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefinetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol.

Illustrative antivirals include abacavir sulfate, acyclovir sodium, amantadine hydrochloride, amprenavir, eidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanamivir, and zidovudine.

Non-limiting examples of amebicides or antiprotozoals include atovaquone, chloroquine hydrochloride, chloroquine phosphate, metronidazole, metronidazole hydrochloride, and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, and terbinafine hydrochloride. Non-limiting examples of antimalarials include chloroquine hydrochloride, chloroquine phosphate, doxycycline, hydroxychloroquine sulfate, mefloquine hydrochloride, primaquine phosphate, pyrimethamine, and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

It is also known that medical treatments that target rapidly dividing cells and/or disrupt the cell cycle or cell division (e.g., chemotherapy and radiation therapy) are immunocompromising since cells of the immune system including hematopoietic cells are destroyed or substantially reduced in number, thus leading to a state of immunosuppression characterized by neutropenia, agranulocytosis, thrombocytopenia and/or anemia. Accordingly, the present invention finds particular utility in the treatment or prophylaxis of any one or more of these conditions that manifest from a medical treatment as broadly noted above.

Anemia, thrombocytopenia, neutropenia and agranulocytosis are frequently defined in terms of laboratory measurements indicating a reduced hematocrit (volume percent), a reduced platelet count (per mm³), a reduced neutrophil count (per mm³), a reduced total granulocyte (i.e., neutrophils, basophils and eosinophils) or white blood cell count (per mm³), respectively. Methods of determining these values are well known in the art, including automated as well as manual methods. The lower limits of normal for hematocrits and platelet counts in healthy non-pregnant humans is somewhat variable, depending on the age and sex of the subject, method of determination, and the norms for the laboratory performing the measurements. Generally, however, an adult human subject is said to have anemia when the hematocrit is less than about 37-40%. Likewise, generally an adult human subject is said to have thrombocytopenia when the platelet count is below about 100,000 per mm³. Anemia is also frequently reported in terms of a reduced hemoglobin (g/dL) or red blood cell count (per mm³). Typical lower limits of normal values for these in healthy adult humans are 12-13 g/dL and about 4.1×10⁶ per mm³, respectively. Generally an adult human subject is said to have neutropenia when the neutrophil count falls below 1000 per mm³. Additionally, an adult human is generally said to have agranulocytosis when the total granulocyte cell count falls below 500 cells/mm³. Corresponding values for all these parameters are different for other species.

Hematopoietic disorders such as anemia, thrombocytopenia, neutropenia and agranulocytosis are also frequently associated with clinical signs and symptoms in relation to their degree of severity. Anemia may be manifested as pallor, generalized fatigue or weakness, reduced exercise tolerance, shortness of breath with exertion, rapid heart rate, irregular heart rhythm, chest pain (angina), congestive heart failure, and headache. Thrombocytopenia is typically manifested in terms of spontaneous or uncontrolled bleeding, petechiae, and easy bruising. Neutropenia is associated with infections, including notably infections from endogenous microbial flora, and lack of inflammation.

Accordingly, the present invention contemplates ancillary combination treatment which employ both the HIF-α potentiating agent/mobilizer combination and an ancillary treatment that treats a hematopoietic disorder as broadly described above. In some embodiments, the ancillary combination treatment will employ a HIF-α potentiating agent/mobilizer combination and a medicament selected from an anemia medicament, a thrombocytopenia medicament, an agranulocytosis medicament or a neutropenia medicament, illustrative examples of which include steroids, inducers of steroids, and immunomodulators.

The steroids include, but are not limited to, systemically administered corticosteroids including methylprednisolone, prednisolone and prednisone, cortisone, and hydrocortisone. Inducers of steroids include, but are not limited to adrenocorticotropic hormone (ACTH).

Corticosteroids inhibit cytokine production, adhesion protein activation, and inflammatory cell migration and activation. The side effects associated with systemic corticosteroids include, for instance, reversible abnormalities in glucose metabolism, increased appetite, fluid retention, weight gain, mood alteration, hypertension, peptic ulcer, and aseptic necrosis of bone. Some side effects associated with longer term use include adrenal axis suppression, growth suppression, dermal thinning, hypertension, diabetes mellitus, Cushing's syndrome, cataracts, muscle weakness, and in rare instances, impaired immune function. It is recommended that these types of compounds be used at their lowest effective dose.

Commonly used anemia drugs which are currently on the market or in development include recombinant human EPO (EPOGEN; PROCRIT), preparations of iron (ferrous and ferric, CHROMAGEN; FEOSOL; INFED; IROSPAN; NEPHRO-FER; NEPHRO-VITE; NIFEREX; NU-IRON; SLOW FE), vitamin B12, vitamin B6, folic acid (CHROMAGEN; FERRO-FOLIC; NEPHRO-FER; NIFEREX), ascorbic acid, certain metabolites of vitamin D (calcitriol and alphacalcidol; CALCIJEX; ROCALTROL), androgens, and anabolic steroids (ANADROL), carnitine. In a specific embodiment the anemia medicament is recombinant EPO.

Drugs in common usage or development for the treatment of thrombocytopenia include glucocorticoids (prednisolone; prednisone; methylprednisolone; SOLUMEDROL), recombinant TPO, recombinant MGDF, pegylated recombinant MGDF, and lisophylline. In a specific embodiment the thrombocytopenia medicament is recombinant TPO.

Drugs in common usage or development for the treatment of neutropenia include glucocorticoids (prednisolone; prednisone; methylprednisolone; SOLUMEDROL), immunoglobulin G (SANDOGLOBULIN, IVEEGAM, GAMMAR-P, GAMIMNE N, GAMMAGARD S/D), androgens, recombinant IFN-γ (ACTIMMUNE), and uteroferrin. Antibiotics are frequently administered in association with neutropenia medicaments to treat or reduce the risk of infection.

As noted above, the present invention encompasses co-administration of a HIF-α potentiating agent/mobilizer combination in concert with an additional or ancillary agent. It will be understood that, in embodiments comprising administration of the HIF-α potentiating agent/mobilizer combination with other agents, the dosages of the active agents in the combination may on their own comprise an effective amount and the additional agent(s) may further augment the therapeutic or prophylactic benefit to the patient. Alternatively, the HIF-α potentiating agent/mobilizer combination and the additional agent(s) may together comprise an effective amount for preventing or treating the immunocompromised condition or infection. It will also be understood that effective amounts may be defined in the context of particular treatment regimens, including, e.g., timing and number of administrations, modes of administrations, formulations, etc.

In some embodiments, the present invention contemplates administering a high dose of the medical treatment that induces the immunocompromised condition, without inducing side effects or inhibiting the induction of side effects. Ordinarily, when medical treatments such as chemotherapy and radiotherapy are administered in a high dose, a variety of side effects can occur, including the induction of the immunocompromised condition and infection. As a result of these side effects, the medical treatment is not administered in such high doses. In accordance with the present invention, such high doses of medical treatment (e.g., a higher dose of chemotherapeutic agent or radiation) which ordinarily induce side effects can be administered without inducing the side effects as long as the subject also receives concurrent administration of a HIF-α potentiating agent and at least one mobilizer of hematopoietic stem cells and/or progenitor cells. The type and extent of the side effects ordinarily induced by the medical treatment will depend on the particular treatment used.

Suitably, the HIF-α potentiating agent/mobilizer combination, and optionally the ancillary treatment, are administered on a routine schedule. Alternatively, the ancillary treatment may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the HIF-α potentiating agent on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve concurrent administration of HIF-α potentiating agent and the mobilizer on a daily basis for the first week, followed by a monthly basis for several months, and then every three months after that. Any particular combination would be covered by the routine schedule as long as it is determined ahead of time that the appropriate schedule involves administration on a certain day.

Additionally, the present invention provides pharmaceutical compositions for stimulating or enhancing mobilization of hematopoietic stem cells and/or progenitor cells, or for stimulating or enhancing hematopoiesis, or for stem cell transplantation or for treating or preventing an immunocompromised condition, including one that results from a medical treatment as broadly described above. The pharmaceutical compositions include an HIF-α potentiating agent and at least one mobilizer of hematopoietic stem cells and/or progenitor cells, optionally formulated in a pharmaceutically acceptable carrier. In specific embodiments, the pharmaceutical compositions comprises a PHI (e.g., a small molecule PHI including ones selected from compounds according to any one of formulae I-IX) and one or both of a colony-stimulating factor (e.g., G-CSF or a variant, derivative or analog thereof) and a CXCR4 antagonist (e.g., a small molecule CXCR4 antagonist)

The pharmaceutical composition may include an ancillary or additional medicament as broadly described above. In some embodiments, the HIF-α potentiating agent and the mobilizer(s) will be present in the pharmaceutical composition in an effective amount for preventing or treating an immunocompromised condition (e.g., anemia, thrombocytopenia, or neutropenia). The effective amount for preventing or treating the immunocompromised condition is that amount which completely or partially prevents the development of, prevents the worsening of, or treats the established existence of, the immunocompromised condition. In some instances, the effective amount for preventing or treating immunocompromised condition completely or partially prevents or treats clinical symptoms of that condition.

In addition to clinical outcomes measured in terms of physiology, in vitro assays measuring erythrocyte, platelet, granulocyte and total white blood cell counts may be used in determining a therapeutically effective amount of a particular HIF-α potentiating agent. These methods are standard medical laboratory techniques that are well known in the art. In common practice such measurements may be made by automated cell counting devices designed for that purpose, or they may be performed manually. Manual counts may be more accurate than automated counts when cell counts are particularly low.

The compositions of the invention are administered in pharmaceutically acceptable solutions, which may routinely contain pharmaceutically acceptable concentrations of salt, buffering agents, preservatives, compatible carriers, adjuvants, and optionally other therapeutic ingredients. The HIP-α potentiating agent, in particular, may be formulated in a pharmaceutically acceptable solid form, e.g., tablet, capsule, caplet, powder, pill, etc. Depending on the specific conditions being treated, the formulations may be administered systemically or locally. Techniques for formulation and administration may be found in Goodman and Gilman's: The Pharmacological Bases of Therapeutics, 8th Ed., supra; Remington's Pharmaceutical Sciences, 17th ed., Mack Publishing Co., supra; Pharmaceutical Dosage Forms, Parenteral Medications Dekker, N.Y. supra; Pharmaceutical Dosage Forms: Tablets Dekker, N.Y. supra; and Pharmaceutical Dosage Forms: Disperse Systems Dekker, N.Y, supra. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the active agents or drugs of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

The drugs can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active agents in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active agents with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more drugs as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or dragee coatings for identification or to characterize different combinations of active agent doses.

Pharmaceutical which can be used orally include push-fit capsules made of gelatin, as well as soft, sealed capsules made of gelatin and a plasticizer, such as glycerol or sorbitol. The push-fit capsules can contain the active agents in admixture with filler such as lactose, binders such as starches, or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added.

Dosage forms of the drugs of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes or microspheres.

The drugs of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of an active agent, which achieves a half-maximal inhibition in activity of a PHD polypeptide). Such information can be used to more accurately determine useful doses in humans.

Toxicity and therapeutic efficacy of such drugs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p1).

Alternately, one may administer the compound in a local rather than systemic manner, for example, via injection of the compound directly into a tissue, which is preferably subcutaneous or omental tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

### Compound X Synergizes with G-CSF to Enhance HSC Mobilization

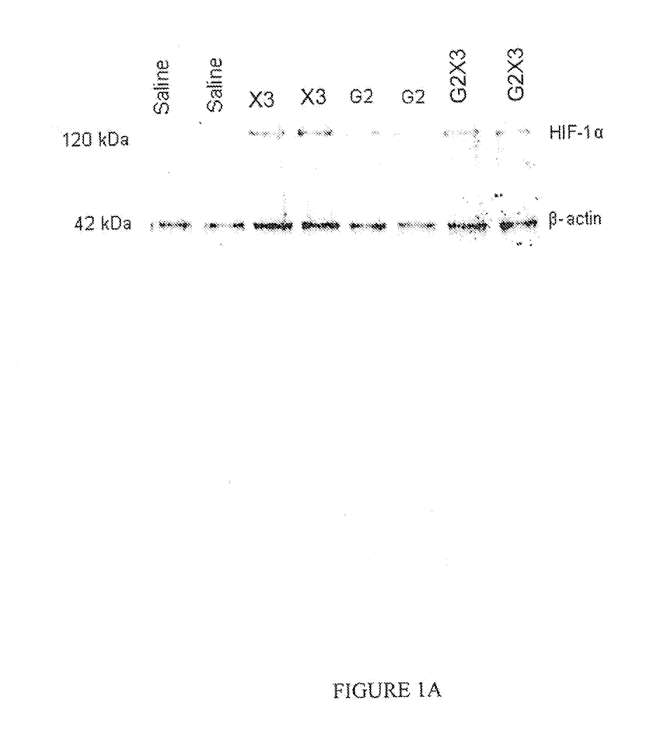

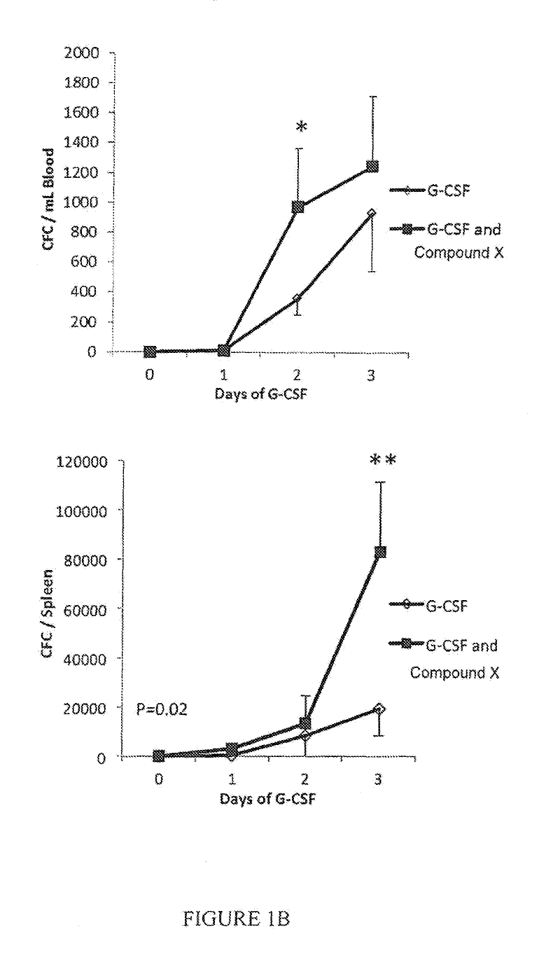

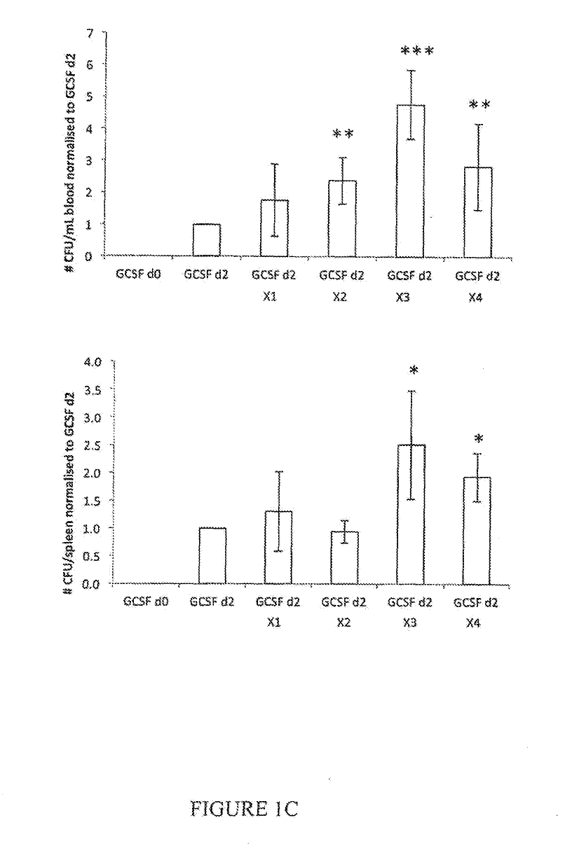

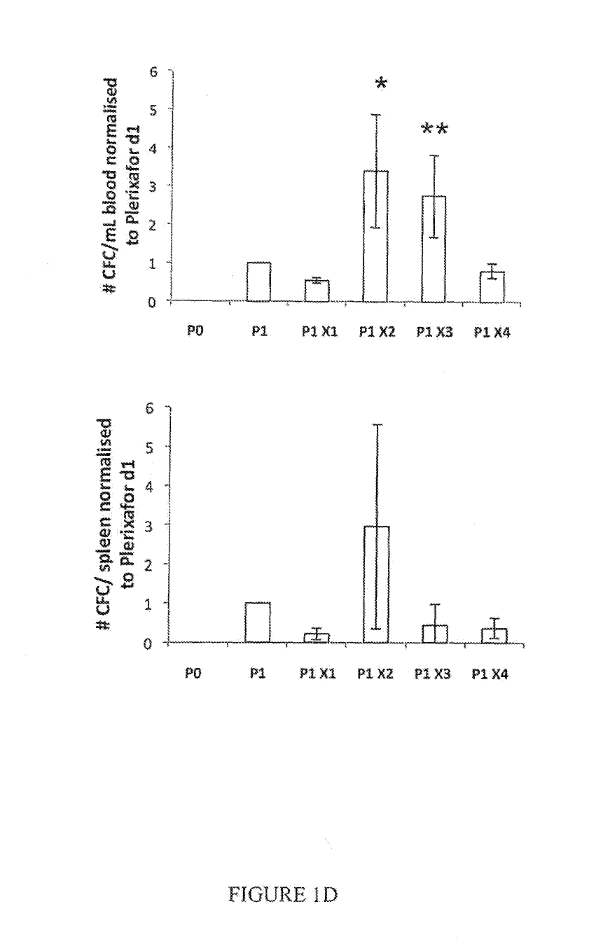

To evaluate whether pharmacological stabilization of HIF-1α protein would enhance HSC mobilization in response to G-CSF, C57Bl/6 mice were treated with recombinant human G-CSF (rhuG-CSF) twice daily for 1-3 days together with a PHD inhibitor, Compound X, daily for 3 days. Pharmacological stabilization of HIF-1α protein was demonstrated in bone marrow leukocytes treated with Compound X (FIG. 1A). Additionally, treatment with Compound X in combination with G-CSF significantly increased stabilization of HIF-1α protein compared to G-CSF alone. Mobilization of colony-forming cells (CFCs) to the blood and spleen were measured (FIG. 1B). Addition of Compound X to G-CSF treatment resulted in a 2.5-fold increase in CFCs mobilized per mL blood following 2 days of G-CSF, and a 6-fold increase of CFCs mobilized to the spleen following 3 days of G-CSF. Of note, Compound X alone for 3 days did not induce CFC mobilization into the blood. These results demonstrate that Compound X synergizes with G-CSF on HSPC mobilization. To assess the optimum duration of Compound X treatment, Compound X was administered for 1-4 days and G-CSF for the last 2 days before tissue sampling (FIG. 1C). CFC mobilization into blood and spleen in response to 2 days of G-CSF progressively increased with the duration of Compound X treatment, peaking at day 3 of Compound X administration.

### Example 2

### Compound X Synergizes with Plerixafor to Enhance CFC Mobilization

The present inventors next evaluated the ability of Compound X to synergize with Plerixafor to enhance mobilization. Since mobilization with Plerixafor peaks 1 h post-injection (7), this time-point was used together with daily administration of Compound X for 1-4 days prior sacrifice (FIG. 1D). Similar to G-CSF, the number of CFC mobilized per mL blood in response to Plerixafor, was significantly increased by a 2 to 3 day treatment with Compound X demonstrating synergy between CXCR4 inhibition and PHD inhibition. As mobilization in response to Plerixafor is very rapid, there was no significant mobilization of HSPC to the spleen 1 hr following Plerixafor treatment with or without Compound X (FIG. 1D).

### Example 3

### PHD Inhibition Synergizes with the Combination of G-CSF and Plerixafor

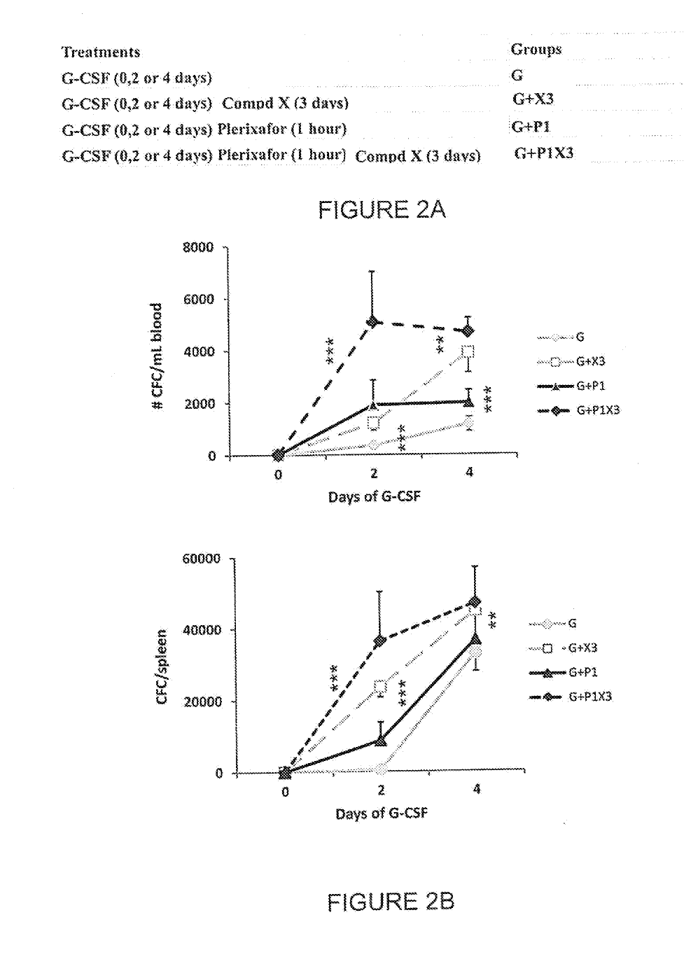

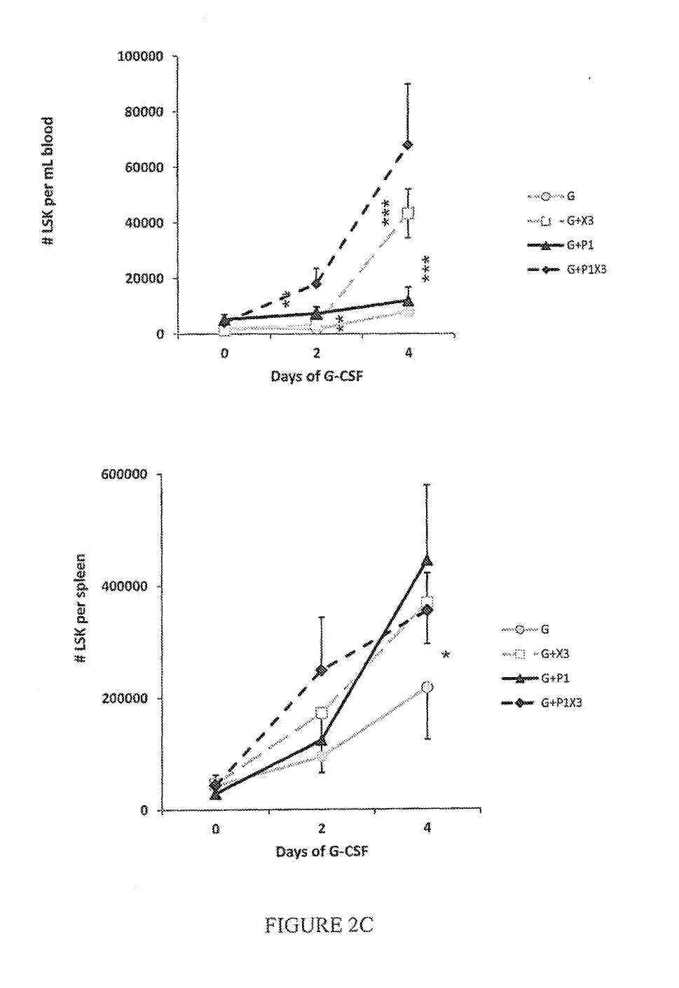

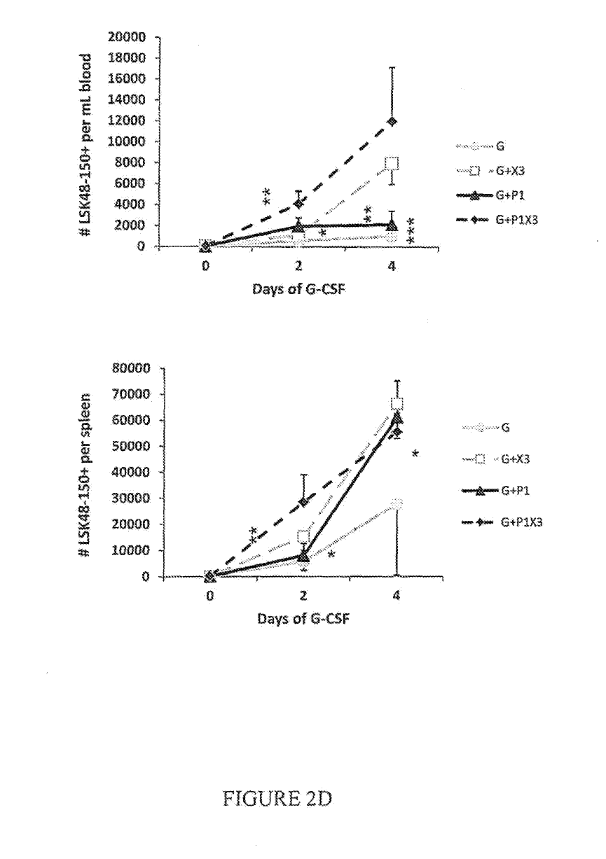

Since Compound X synergized with both G-CSF and Plerixafor separately, the combined effects of G-CSF, Plerixafor and Compound X were next assessed on mobilization. This was divided into two experiments in which G-CSF was administered for 2 days in one experiment and 4 days in the other (FIG. 2). C57BL/6 mice were in 4 treatment groups in both experiments: (G) 250 μg/kg/day G-CSF alone; (G+X3) G-CSF+20 mg/kg/day Compound X for 3 days; (GPI) G-CSF together with 16 mg/kg Plerixafor 1 hour prior harvest, (G+P1X3) G-CSF together with Plerixafor and Compound X with same dosing as above. Mobilization of CFCs, phenotypic Lin⁻CD41⁻Sca1⁺Kit⁺ HSPCs and Lin⁻CD41⁻ Sca1⁺Kit⁺CD48⁻CD150⁺ HSCs were measured in blood and spleens. Mice in the G+X3 group (G-CSF+Compound X) mobilized CFC to the blood 4-fold and the spleen 47-fold more than mice mobilized with G-CSF alone for 2 days (p<0.001; FIG. 2B). Following 4 days treatment with G-CSF, the addition of Compound X resulted in a 4-fold increase in the number of CFC/mL blood (P<0.001) and a further significant increase in the number of CFC/spleen (P<0.01; FIG. 2B). Compound X alone had no mobilizing effect (data not shown). Expectedly, Plerixafor enhanced mobilization of CFC 10-fold in response to 2 days treatment with G-CSF (p<0.005). Most interestingly, addition of Compound X further increased by 2.5-fold the mobilization of CFC/mL into blood and by 4-fold the mobilization of CFC/spleen in response to 2 days G-CSF plus Plerixafor (p<0.005; FIG. 2B). Addition of Compound X also boosted by 2.5-fold the mobilization of CFC/mL blood in response to 4 days G-CSF plus Plerixafor (p<0.005; FIG. 2B).

Mice mobilized with G-CSF for 2 days together with Compound X increased the number of phenotypic HSCs 3-fold while the number of Lin⁻Sca1+Kit⁺ HSPCs doubled in the blood and tripled spleen compared to mice mobilized with G-CSF alone (p<0.005; FIG. 2c-d). Furthermore, the addition of Compound X together with G-CSF and Plerixafor boosted mobilization of phenotypic HSCs 3-fold and HSPCs 2.5-fold in the blood and 4-fold in the spleen following 2 days G-CSF plus Plerixafor (P<0.01; FIG. 2C-D). The addition of Compound X to G-CSF and Plerixafor also dramatically increased the number of phenotypic HSCs and HSPCs in the blood following 4 days treatment with G-CSF and Plerixafor (P<0.001; FIG. 2C-D).

This synergistic increase in HSC mobilization was further confirmed in long-term competitive repopulation assays following transplantation of 20 μL mobilized blood in competition with 2×10⁵ BM cells from congenic donors. CD45.2/CD45.1 chimerism showed that combination of 2 days G-CSF+Plerixafor+Compound X (G2P1X3) mobilized competitive repopulating HSC 6-fold more than G-CSF+Plerixafor (02P1, p<0.01). The number of mobilized long-term competitive repopulating HSC (measured 16 weeks post-transplant) doubled after treatment with 2 days G-CSF+1 h Plerixafor+3 days Compound X (G2P1X3) compared to G-CSF+Plerixafor (G2P1, p<0.05; FIG. 3A). Most interestingly, this increase in the number of mobilized repopulating units with the addition of Compound X was even further pronounced after treatment with G-CSF for 4 days prior to transplant. The addition of Compound X to G-CSF (G4X3) increased 6-fold the number of mobilized repopulating units compared to G-CSF (G4) alone (P<0.001; FIG. 3B). The combination of Compound X with G-CSF and Plerixafor (G4P1X3) increased mobilization of repopulating units 3-fold compared to G-CSF+Plerixafor (G4P1) alone (P<0.001; FIG. 3B). These data demonstrate that Compound X synergistically enhances the mobilization of transplantable HSCs in response to the already potent combination of G-CSF with Plerixafor.

### Example 4

### Deletion of HIF1A Gene in HSPCs Impairs their Mobilization

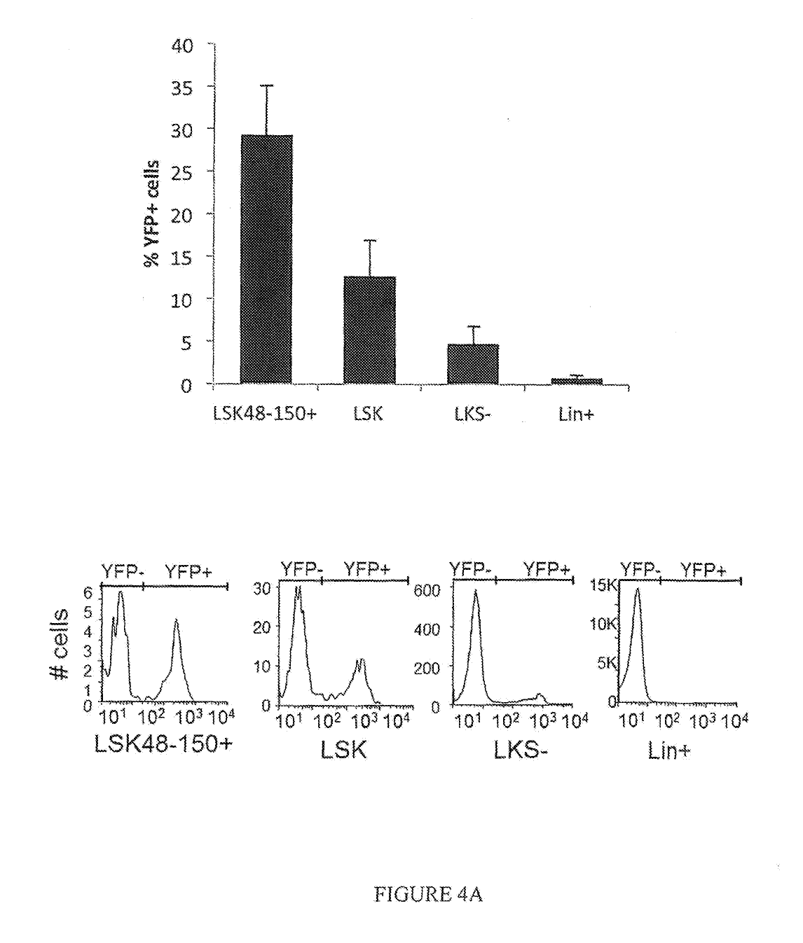

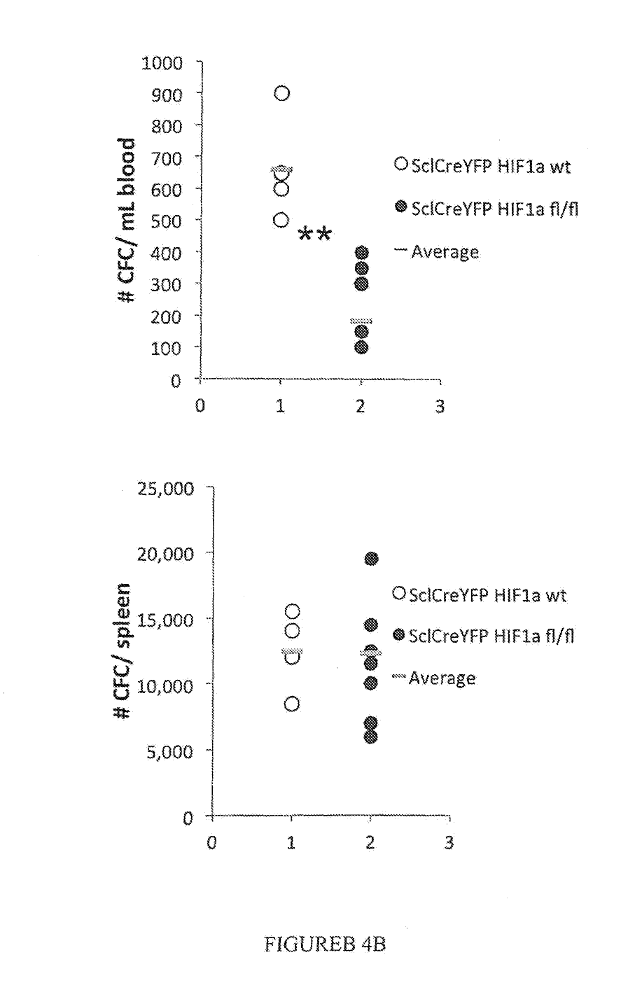

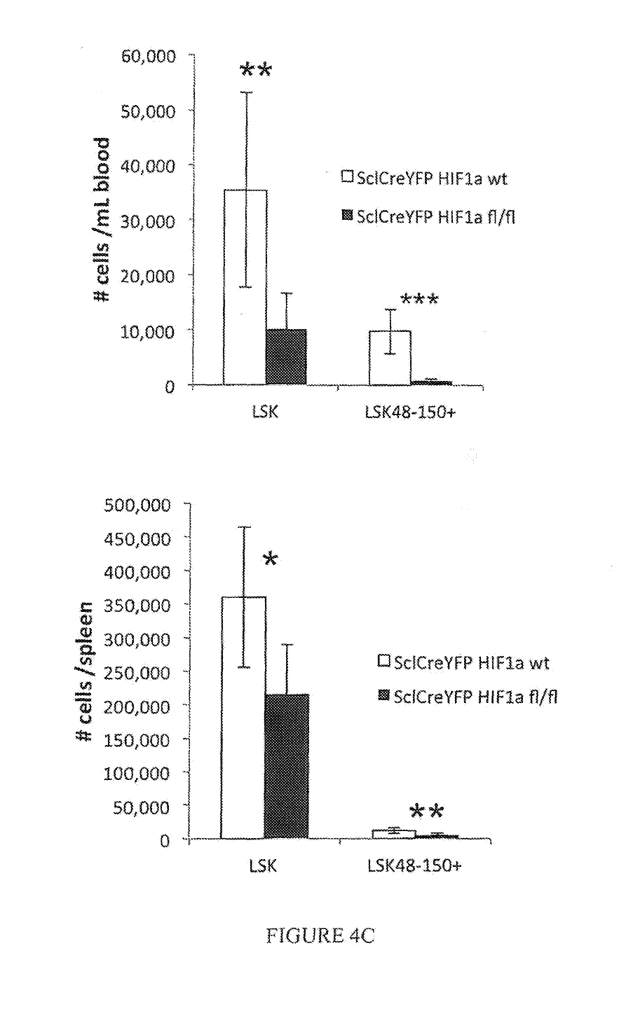

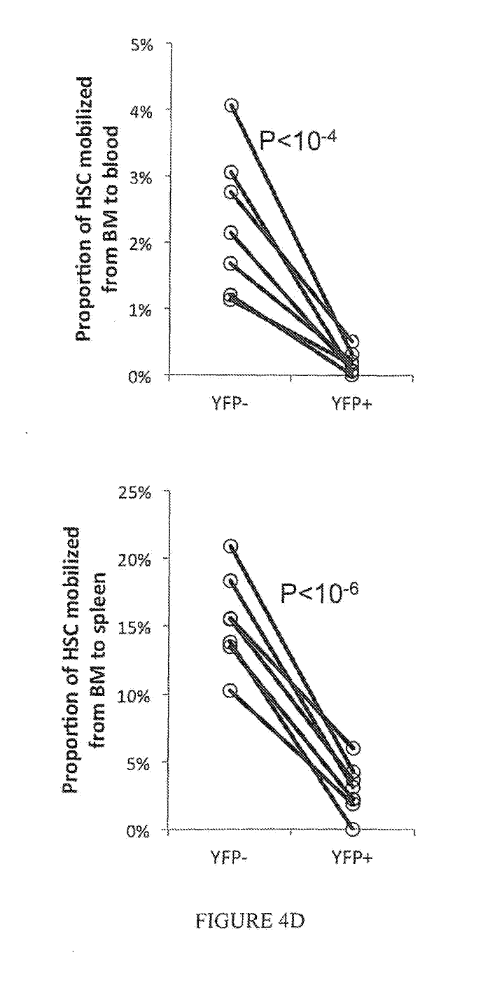

As Compound X was found by the present inventors to stabilize HIF-1α protein in bone marrow HSPCs, it was decided to further investigated the role of HIF-1α in HSPC mobilization in response to G-CSF. A mutant strain was first established in which both Hif1a gene alleles are floxed, together with a Cre-inducible YFP reporter knocked in the Rosa26 gene trap locus R26R^{YFP} (10) together with a tamoxifen-inducible Cre recombinase (CreER fusion protein) specifically expressed in HSC under the control of a HSC specific Scl gene enhancer fragment (SclCreER)(11). By measuring expression of the Cre-inducible YFP reporter by flow cytometry, a 3 day tamoxifen treatment of these Hif1a^{fl/fl} R26R^{YFP/YFP} SclCreER mice caused Cre activation in 30±9% of Lin⁻Sca1⁺Kit⁺CD48⁻CD150⁺ HSCs, 10±5% Lin⁻Sca1⁺Kit⁺ HSPCs (FIG. 4A) but was virtually undetected in Lin⁻Sca1⁻Kit⁺ myeloid progenitors or lineage-positive cells.

Cohorts of Hif1a^{fl/fl} R26R^{YFP/YFP} SclCreER mice and control Hif1a^{WT/WT} R26R^{YFP/YFP} SclCreER mice were induced with tamoxifen for 3 days and then 3 days with G-CSF beginning on the last day of tamoxifen induction. Tissues were harvested 24 hours following the last G-CSF injection. Deletion of Hif1a gene in HSPCs significantly reduced mobilization of CFCs (P<0.01; FIG. 4B), HSPCs (P<0.05) and HSCs (P<0.02) to the blood and spleen in Hif1a^{fl/fl} R26R^{YFP/YFP} SclCreER mice compared to control Hif1a^{WT/WT} R26R^{YFP/YFP} SclCreER mice treated with tamoxifen and G-CSF (FIG. 4C). As Hif1a was deleted from approximately 30% of HSCs in Hif1a^{fl/fl} R26R^{YFP/YFP} SclCreER mice treated with tamoxifen and G-CSF, we took advantage of this mosaic deletion to calculate within each individual mouse the proportion of YFP⁻ (Hif1a gene not deleted) and YFP⁺ (Hif1a deleted) Lin⁻Sca1⁺Kit⁺ HSPCs mobilized to blood or spleen versus the number of Hif1a-intact or Hif1a-deleted HSPCs remaining within the bone marrow. Within each individual mouse, the proportion of YFP⁺ Hif1a-deleted HSPCs that left the bone marrow to mobilize into blood or spleen was significantly lower than the proportion of YFP⁺ Hif1a-intact HSPCs (FIG. 4D). This further demonstrates that deletion of the Hif1a gene in HSPCs severely impairs their mobilization in response to G-CSF.

### Example 5

### HIF-1a Expression in Osteoprogenitors is Necessary for Optimal HSPC Mobilization

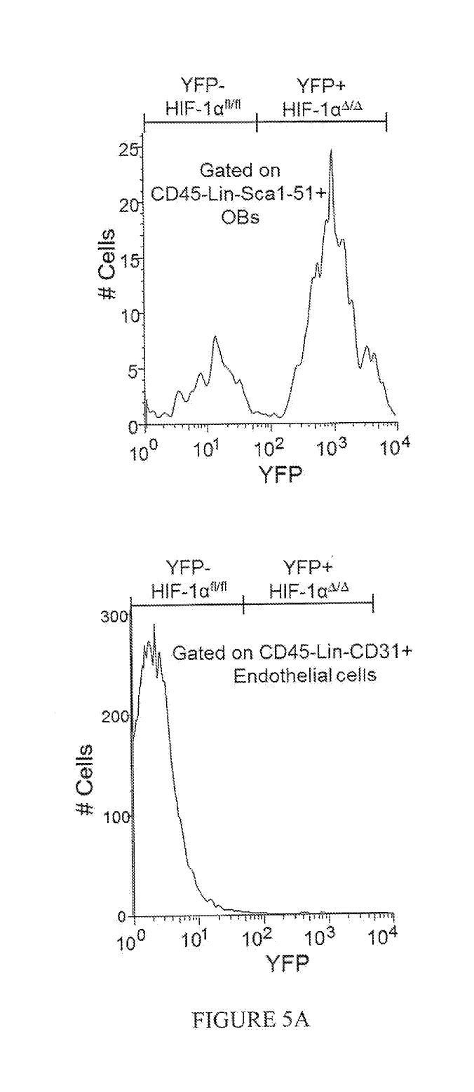

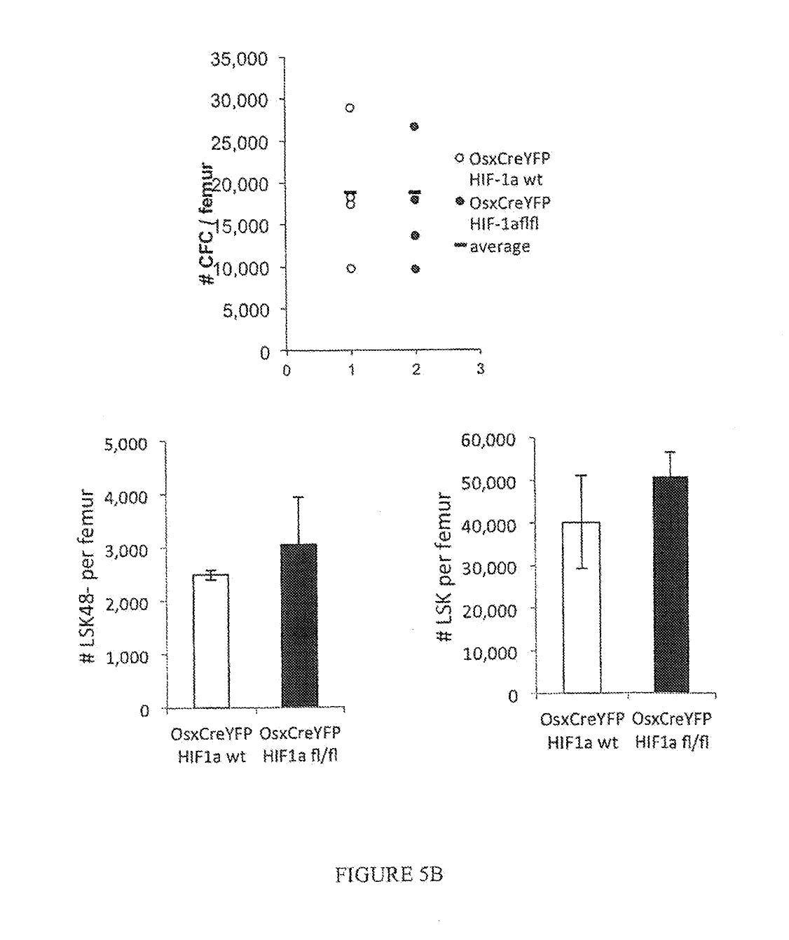

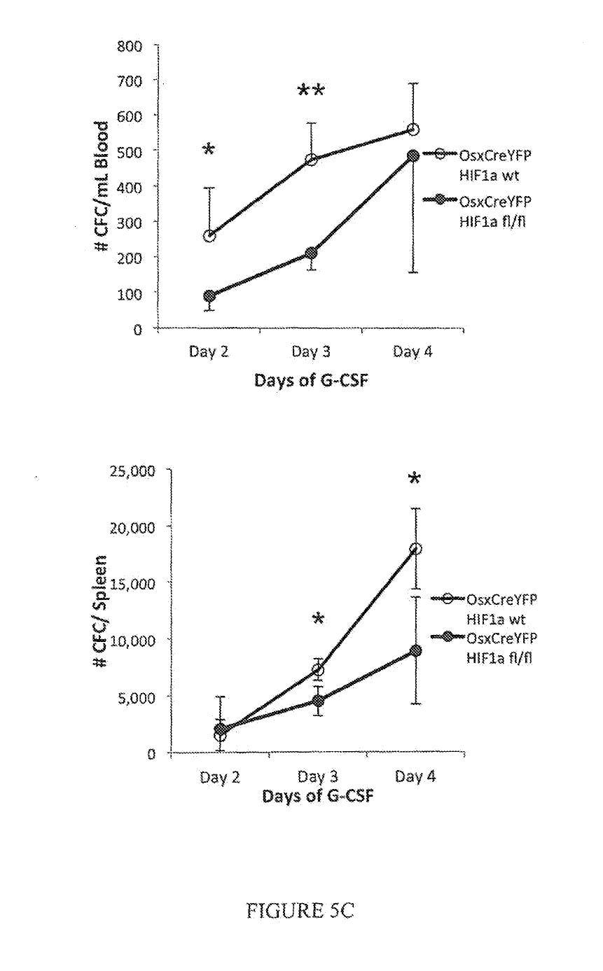

As osteoprogenitors and their progenies play an important role in HSC retention within the BM and mobilization in response to G-CSF (6, 12-15), Hif1a^{fl/fl} osxYFPCre mice were generated with conditional deletion of the Hif1a gene in osteoprogenitors expressing osterix (Sp7 gene) (16). Compared to control Hif1a^{fl/fl} osxYFPCre mice, Hif1a^{fl/fl} osxYFPCre mice had delayed CFC mobilization into blood and spleen at early time-points of G-CSF administration (FIGS. 5A, 5B, 5C). This demonstrates that the effect of HIF-1α in HSPC mobilization is not entirely HSPC autonomous but also requires expression in cells forming the niche such as osteoprogenitors and osteoblasts.

### Example 6

### Compounds A, B and C Enhance HSC Mobilization

Additional HIF-α potentiating agents in the form of PHD inhibitors were tested for their ability to enhance mobilization induced by G-CSF. Compound A, Compound B or Compound C, were tested in male, 7 weeks-old C57BL/6 mice. Mice were injected twice daily sub-cutaneously with rhuG-CSF at a dose of 125 μg/kg per injection for four days. Mice were gavaged with 20 mg/kg Compound A, Compound B, Compound C or vehicle control once daily for three days beginning on day 1 of the experiment.

At the end of treatment, blood was harvested by cardiac puncture, mice were sacrificed by cervical dislocation and spleens harvested. Femurs were removed, cleaned and the bone marrow was flushed with 1 mL PBS+10% newborn calf serum (NCS).

The number of phenotypic hematopoietic stem and progenitor cells remaining in the bone marrow following treatment was measured by flow cytometry using antibodies specific for lineage markers (CD3, CD5, CD45R/B220, F4/80, Gr1, CD41, Ter119), Sca-1, Kit, CD48 and CD150. Similarly, the number of phenotypic hematopoietic stem cells mobilized into the blood and spleen were measured. Colony assays were also performed on blood and spleen to determine the number of colony forming cells per mL of blood and per spleen. All results are presented as mean f standard deviation with n=6 per groups. Levels of significance for differences were calculated using the Student's t-test.

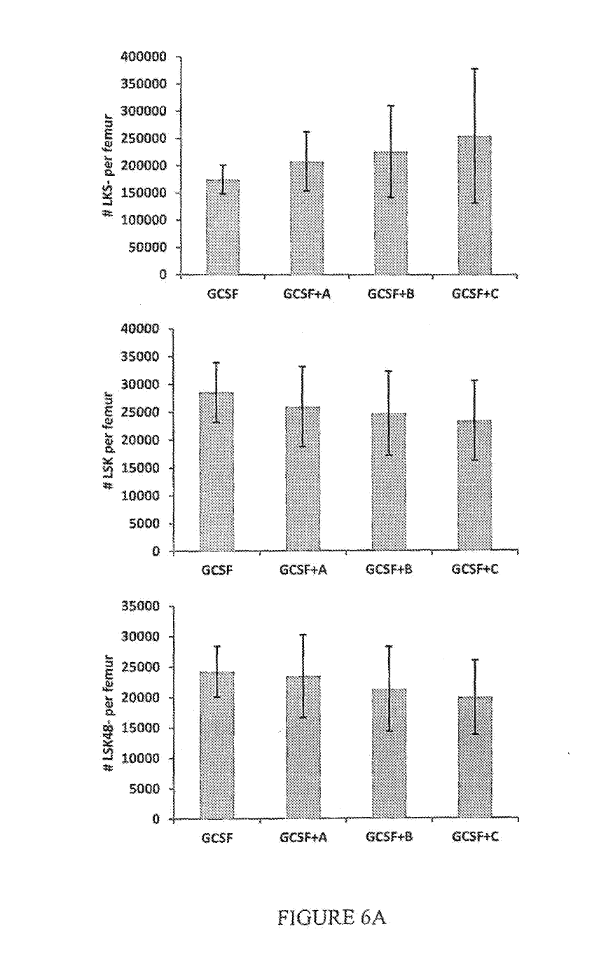

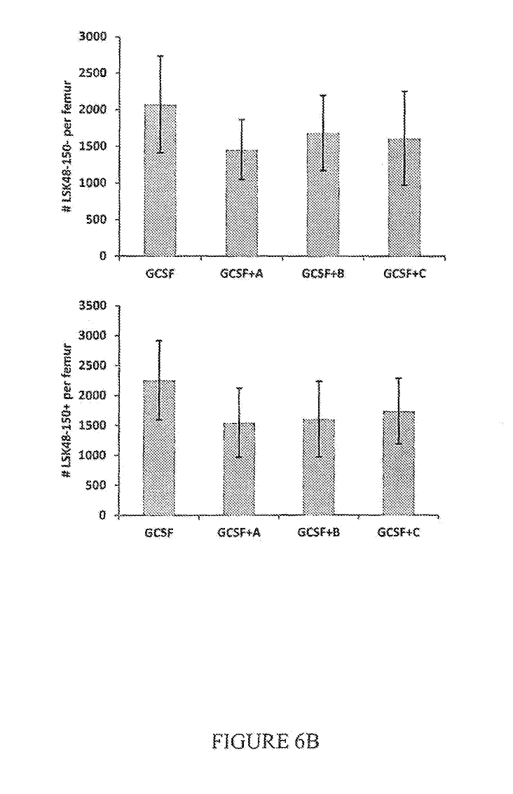

As shown in FIGS. 6A-B, treatment with Compound A, Compound B, or Compound C in combination with G-CSF did not significantly alter the total number of phenotypic hematopoietic stem and progenitor cells (HSPCs) in the bone marrow compared to vehicle control in combination with G-CSF. These results demonstrate that the treatments did not change the phenotype of the mobilized HSCs and HPCs and suggest that the mobilized cells should engraft following transplantation.

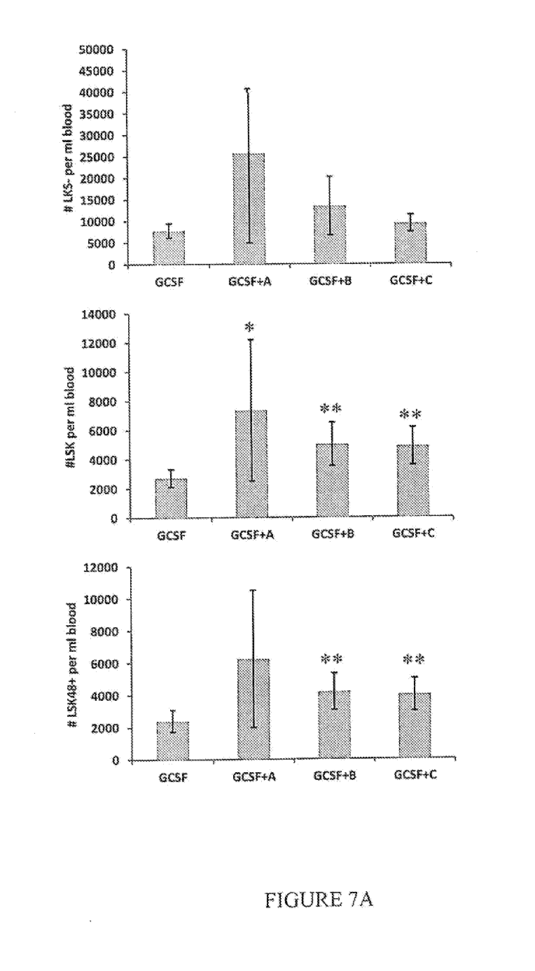

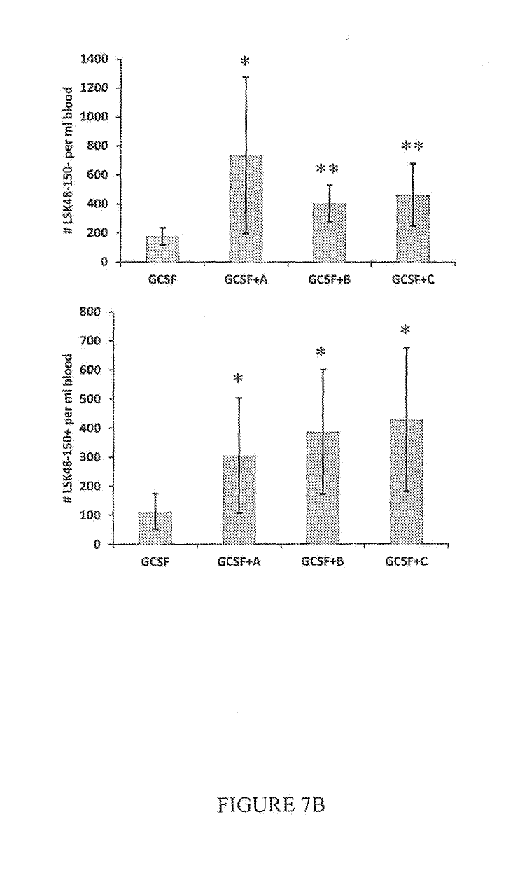

As shown in FIGS. 7A-B, treatment with Compound A, Compound B, or Compound C in combination with G-CSF resulted in a significant increase of approximately 3-fold or greater in the number of phenotypic HSCs (LSK48−150+) and a significant increase of approximately 2-fold or greater in the number of HSPCs (LSK48−150− and LSK) mobilized to the blood compared to vehicle control in combination with G-CSF. Treatment with Compound A, Compound B, or Compound C in combination with G-CSF did not significantly alter the mobilization of myeloid progenitors (LKS−) to the blood compared to vehicle control in combination with G-CSF.

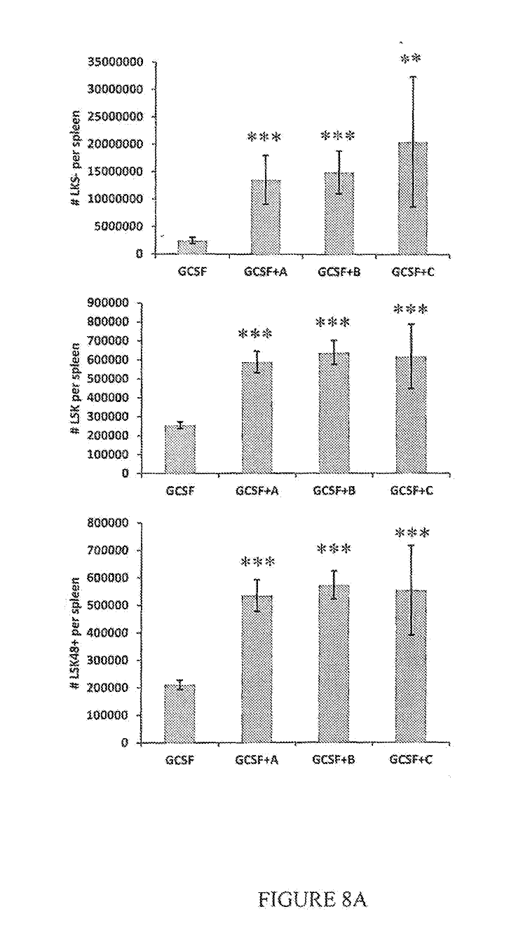

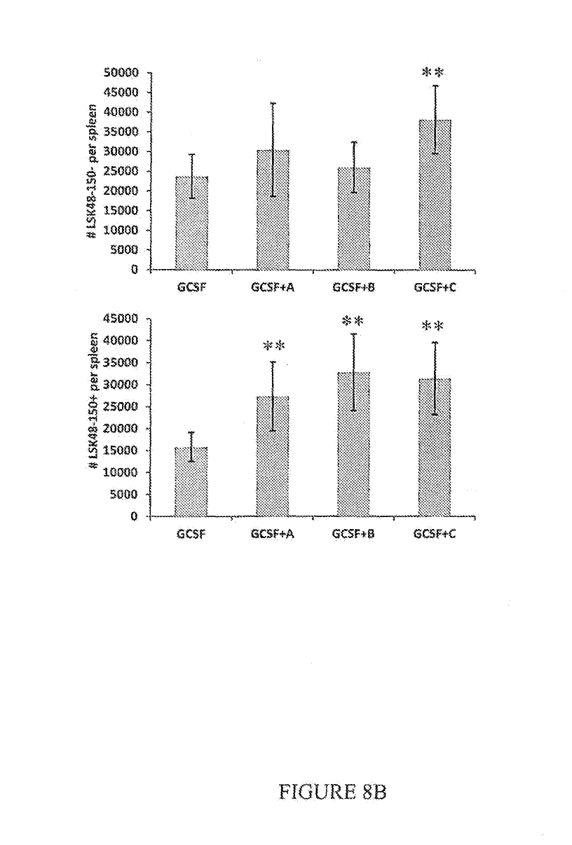

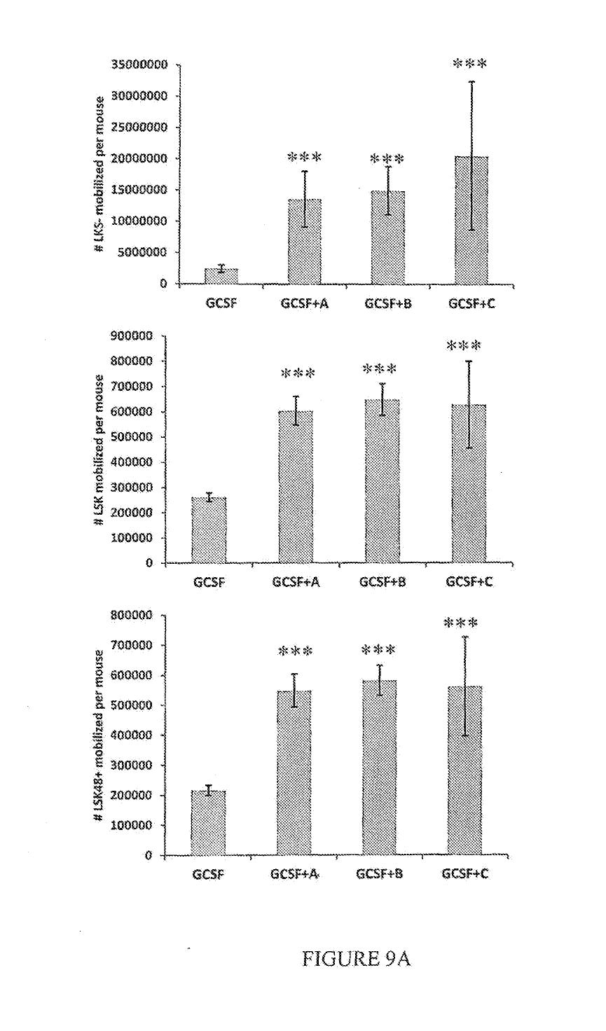

As shown in FIGS. 8A-B, treatment with Compound A, Compound B, or Compound C in combination with G-CSF resulted in a significant increase of approximately 2-fold in the number of phenotypic HSCs (LSK48−150+) and HSPCs (LSK48+ and LSK) mobilized to the spleen compared to vehicle control in combination with G-CSF. Treatment with Compound A, Compound B, or Compound C in combination with G-CSF also significantly increased by approximately 3-fold, the mobilization of myeloid progenitors (LKS−) to the spleen compared to vehicle control in combination with G-CSF.

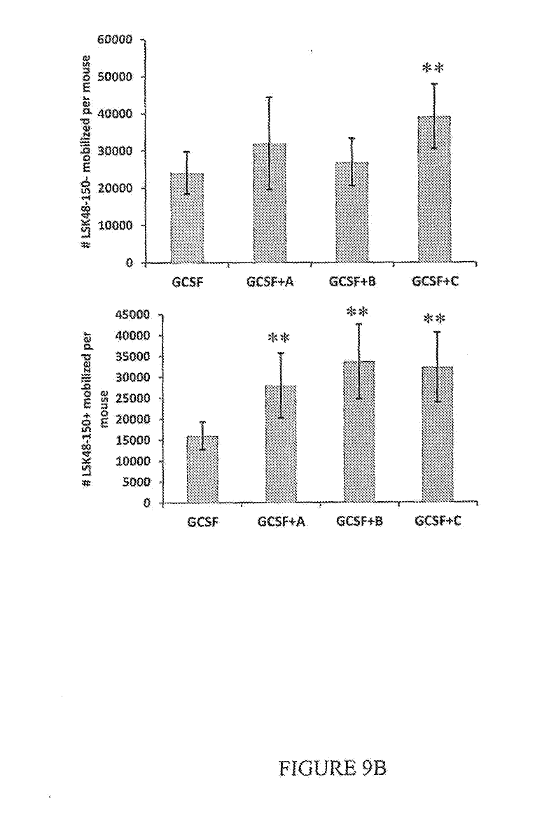

As shown in FIGS. 9A-B, treatment with Compound A, Compound B, or Compound C in combination with G-CSF resulted in a significant increase of approximately 2-fold in the total number of phenotypic HSCs (LSK48−150+) and HSPCs (LSK48+ and LSK) mobilized from the bone marrow in comparison to vehicle control in combination with G-CSF. Additionally, treatment with Compound A, Compound B, or Compound C in combination with G-CSF resulted in a significant increase of approximately 6-fold in the total number of myeloid progenitors (LKS−) mobilized from the bone marrow in comparison to vehicle control in combination with G-CSF.

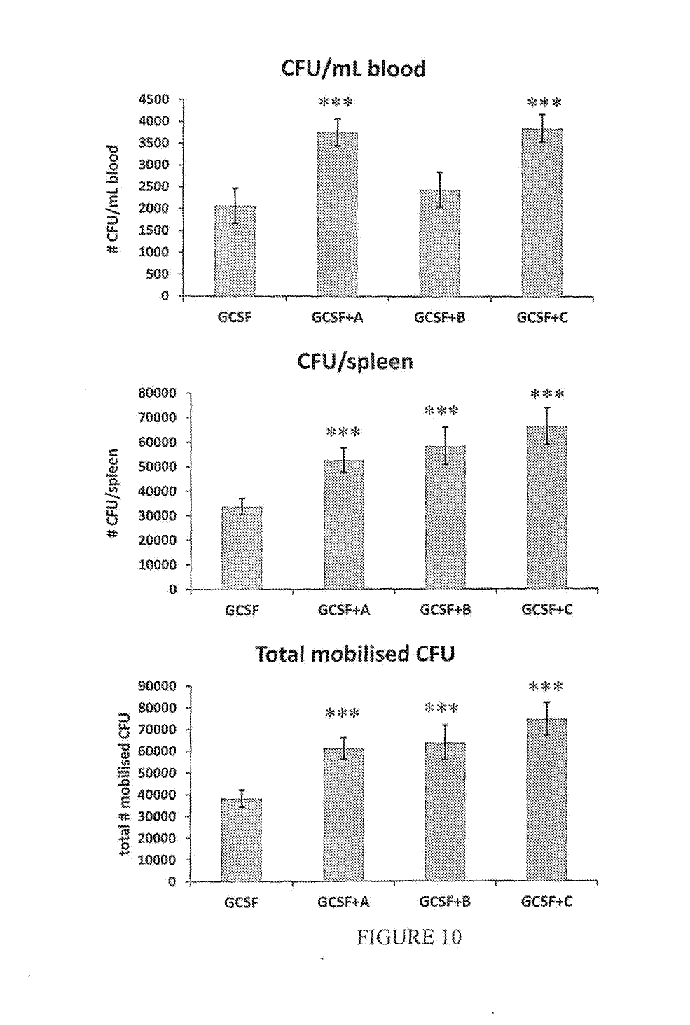

As shown in FIG. 10, treatment with Compound A or Compound C in combination with G-CSF significantly increased the number of colony forming units mobilized from the bone marrow to the blood in comparison to vehicle control in combination with G-CSF. There was no significant change in the number of CFUs mobilized to the blood following treatment with Compound B in combination with G-CSF. This finding is believed to reflect a more rapid induction of mobilization by Compound B compared to the other compounds. Accordingly, the timepoint used in the experiment for blood and tissue collection misses the period of peak induction for Compound B. This hypothesis is supported by the statistically significant number of CFUs mobilized to the spleen for Compound B in combination with G-CSF compared to vehicle and G-CSF as it is known that mobilized stem and progenitor cells are taken up and retained over time by the spleen. Further, the total number of CFUs mobilized by Compound B in combination with G-CSF is comparable to number of CFUs mobilized by Compound A or Compound C in combination with G-CSF.

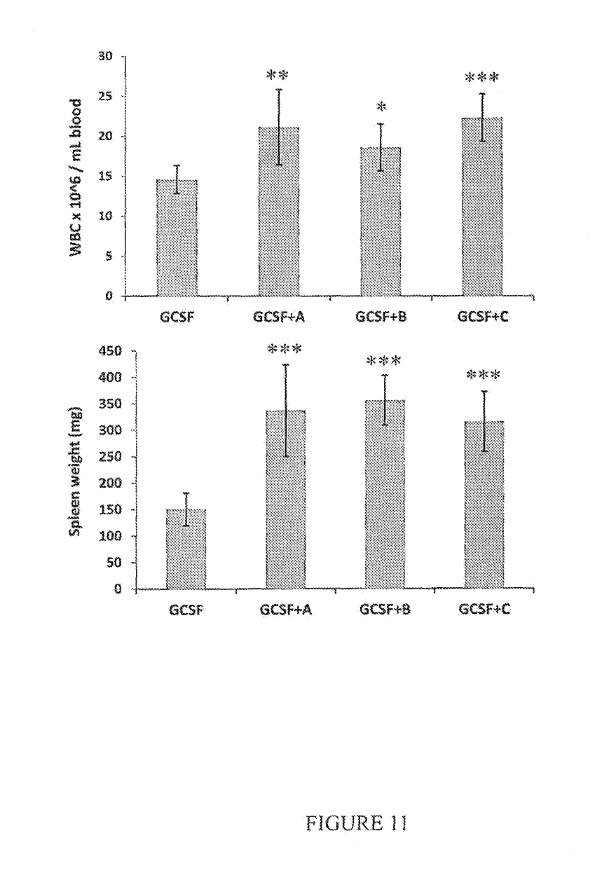

As shown in FIG. 11, treatment with Compound A, Compound B or Compound C in combination with G-CSF resulted in a significant increase in the number of white blood cells per mL of blood compared to vehicle control in combination with G-CSF. Furthermore, treatment with Compound A, Compound B, or Compound C in combination with G-CSF resulted in a significant increase in spleen weight of approximately 2-fold compared to vehicle control in combination with G-CSF.

In conclusion, the results show that the exemplary PHD inhibitors enhanced the mobilization of hematopoietic stem and/or progenitor cells with G-CSF. Significantly elevated levels of phenotypic HSCs and HSPCs were mobilized using the combination of a PHD inhibitor and G-CSF compared to the use of G-CSF alone. Further, the mobilized HSCs and HSPCs were functional and retained the ability to proliferate and differentiate as demonstrated by the CFU assays.

### Example 7

### Compounds D, E and F Enhance HSC Mobilization

Further HIF-α potentiating agents in the form of PHD inhibitors were tested for their ability to enhance mobilization of hematopoietic stem and/or progenitor cells induced by G-CSF treatment. Compounds D, E and F were tested in combination with rhuG-CSF in male, 7 weeks-old C57BL/6 as described in Example 6, above. Compound D was administered at 60 mg/kg, while Compounds E and F were administered at 20 mg/kg. Blood and tissue samples were collected, processed and analyzed by flow cytometry and colony assays as described in Example 6.

Treatment with Compound D, Compound E or Compound F in combination with G-CSF did not significantly alter the total number of HSCs and progenitors in the BM compared to vehicle control in combination with G-CSF.

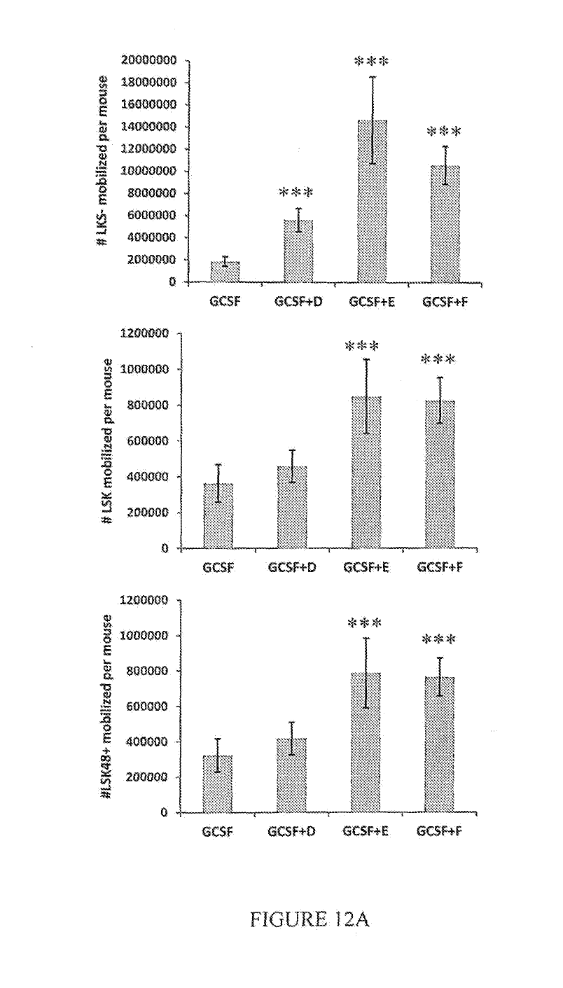

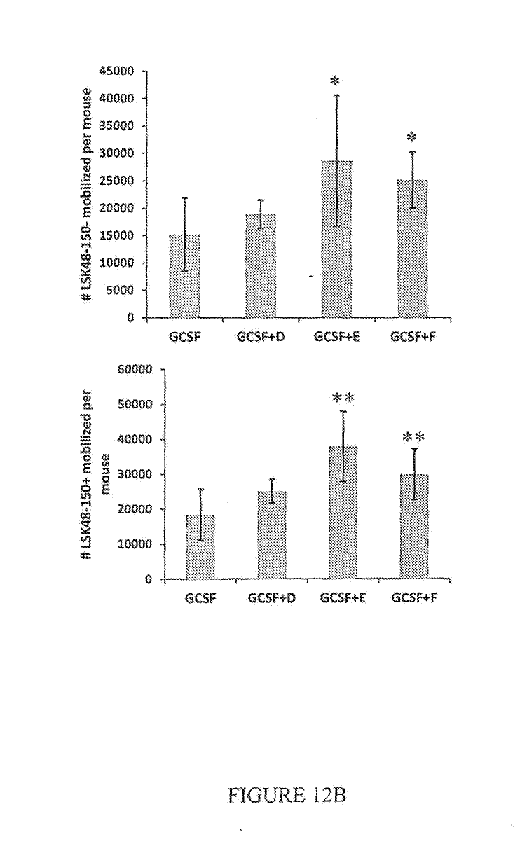

The total mobilization to blood and spleen of phenotypic myeloid progenitors (LKS−) and hematopoietic stem and progenitor cells (LKS+) following treatment with Compound D, Compound E or Compound F in combination with G-CSF is shown in FIGS. 12A-B. Treatment with Compound E or Compound F in combination with G-CSF resulted in a significant increase in the total number of HSCs (LSK48-150+) HSPCs (LSK48+ and LSK) and myeloid progenitors (LKS−) mobilized from the bone marrow in comparison to vehicle control in combination with G-CSF. Compound D in combination with G-CSF significantly increased the total number of mobilized myeloid progenitors (LKS in comparison to vehicle control in combination with G-CSF.

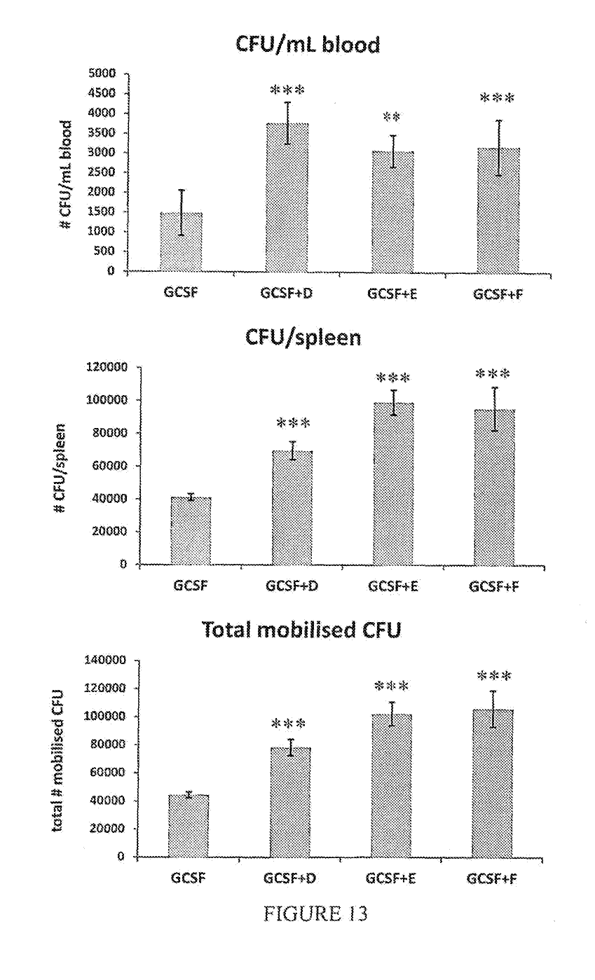

Treatment with Compound D, Compound E or Compound F in combination with G-CSF significantly increased in the number of CFUs mobilized from the bone marrow to the blood and spleen in comparison to vehicle control in combination with G-CSF. See FIG. 13.

Treatment with Compound D or Compound E in combination with G-CSF resulted in a significant increase in the number of white blood cells per mL of blood compared to vehicle control in combination with G-CSF. Furthermore, treatment with Compound D, Compound E or Compound F in combination with G-CSF resulted in a significant increase in spleen weight of approximately 2-fold compared to vehicle control in combination with G-CSF.

In summary, the results obtained with the exemplary PHD inhibitors demonstrated that these PHD inhibitors enhanced the mobilization of phenotypic HSCs and HSPCs induced with G-CSF. Significantly elevated levels of phenotypic HSCs and HSPCs were mobilized using the combination of a PHD inhibitor and G-CSF compared to the use of G-CSF alone. Further, the mobilized HSCs and HSPCs were functional and retained the ability to proliferate and differentiate as demonstrated by the CFU assays.

### Example 8

### Compounds H, J and K Enhance HSC Mobilization

Additional PHD inhibitors were tested for their ability to enhance mobilization induced with G-CST treatment. Compounds H, J and K were tested in combination with G-CSF in male, 7 weeks-old C57BL/6 as described in Example 6, above. Compounds H and J were administered at 60 mg/kg, while Compound K was administered at 100 mg/kg. Blood and tissue samples were collected, processed and analyzed by flow cytometry and colony assays as described in Example 6.

Treatment with Compound H, Compound J or Compound K in combination with G-CSF did not significantly alter the total number of HSCs and progenitors in the BM compared to vehicle control in combination with G-CSF.

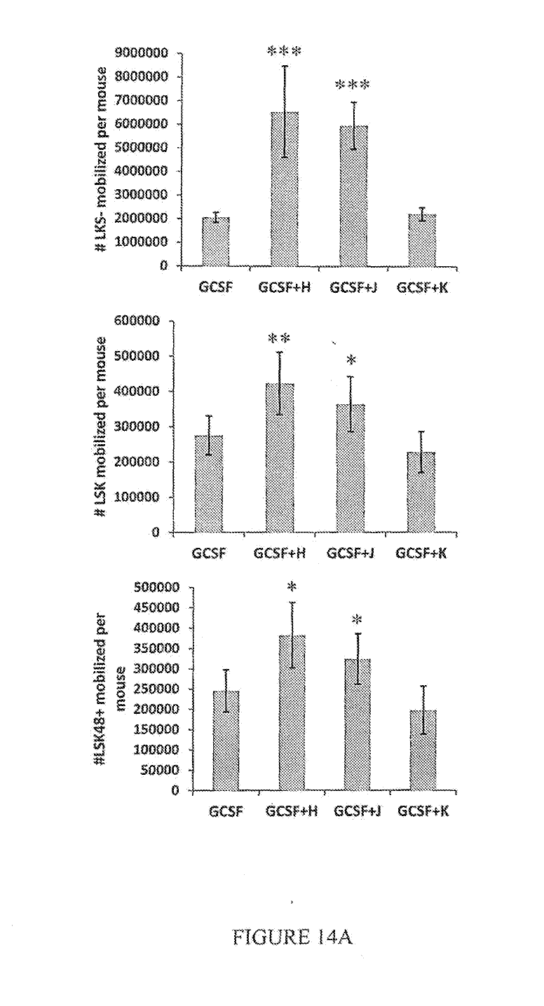

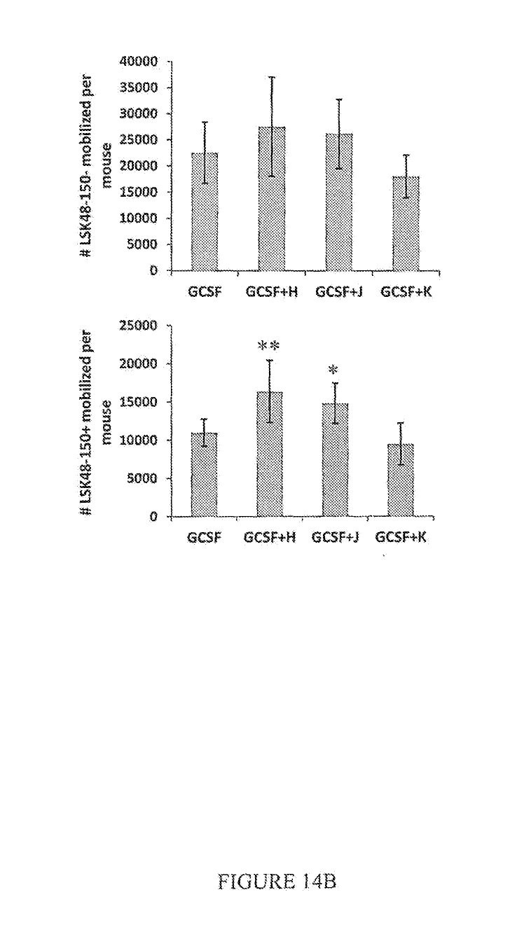

Treatment with Compound H or Compound J in combination with G-CSF resulted in a significant increase in the total number of phenotypic HSCs (LSK48−150+) and HSPCs (LSK48+ and LSK) mobilized blood and spleen in comparison to vehicle control in combination with G-CSF. See FIGS. 14A-B.

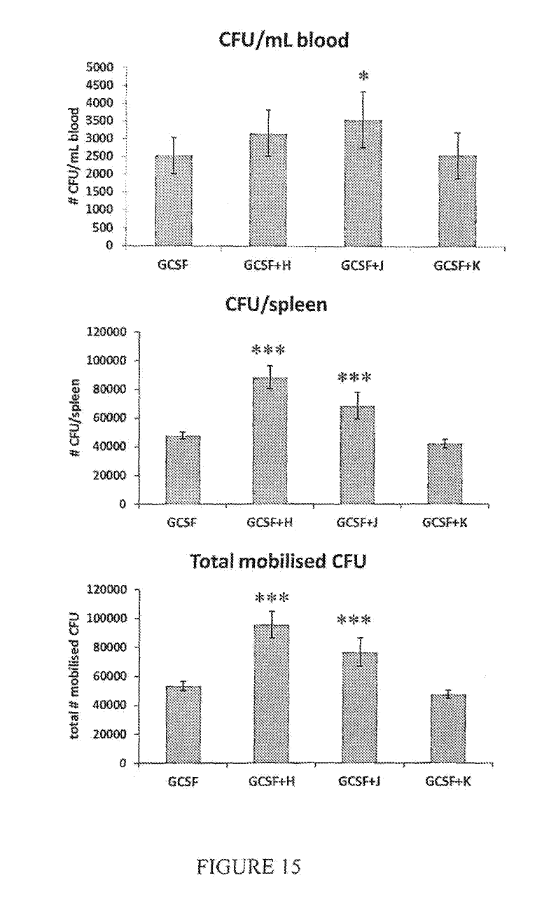

Treatment with Compound J in combination with G-CSF resulted in a significant increase in the number of colony forming units mobilized to the blood compared to vehicle control. See FIG. 15. Treatment with Compound H or Compound J in combination with G-CSF resulted in significant increase in the number of colony forming units mobilized to the spleen and total number (blood and spleen) of mobilized CFUs in comparison to vehicle control in combination with G-CSF. There was no significant change in the number of CFUs mobilized to the spleen or total number of CFUs following treatment with Compound K or vehicle control in combination with G-CSF. The lack of significant mobilization with Compound K in combination with G-CSF may reflect poor bioavailability or the use of a sub-optimal dosing regimen.

The results demonstrate that the exemplary PHD inhibitors enhanced the mobilization of phenotypic HSCs and HSPCs induced with rhuG-CSF treatment. Significantly elevated levels of phenotypic HSCs and HSPCs were mobilized using the combination of PHD inhibitor and G-CSF compared to the use of vehicle control and G-CSF. Further, the mobilized HSCs and HSPCs were functional and retained the ability to proliferate and differentiate as demonstrated by the CFU assays.

### Discussion of the Examples

The present study is the first to explore the in vivo effects of pharmacological stabilization of HIF-1α protein on mobilization of HSC to the blood for transplantation. The present inventors found that pharmacological stabilization of HIF-1α using an inhibitor of HIF prolyl hydroxylase, Compound X, synergized with both G-CSF and Plerixafor to significantly increase the number of HSC mobilized from the bone marrow to the blood. Indeed administration of Compound X in combination with G-CSF and Plerixafor tripled the number of long-term repopulating HSC per mL of blood. Similar results were found with other inhibitors of HIF prolyl hydroxylase (Compounds A, B, C, D, E, F, H, J, and K). Finally, conditional deletion of the Hif1a gene demonstrates that HIF-1α expression in HSC is critical to efficient mobilization in response to G-CSF. Interestingly, the presence of HIF-1α is also required in niche cells such as osteoprogenitors and osteoblasts for optimal mobilization HSC.

The mechanisms by which HIF-1α stabilization synergistically enhances HSC mobilization in response to G-CSF and Plerixafor remain unclear. The results with conditional deletion of Hif1a in HSCs and osteoprogenitors suggest that the dominant effect of HIF-1a protein stabilization on HSC mobilization is cell-autonomous with microenvironment effects important for maximal mobilization.

### Materials and Methods

### Mouse Strains

C57BL/6 mice were purchased from the Australian Resource Centre, Perth Australia. All genetically modified mice had been backcrossed at least 10 times into C56BL/6 background. B6.129-Hif1a^{tm3Rsjo}/J (Hif1a^{flox/flox}) mice were purchased from the Jackson Laboratory (Bar Harbor, Me.). SclCreER transgenic mice expressing a tamoxifen-inducible Cre recombinase driven by a HSC specific Scl gene enhancer (11), B6-Gt(ROSA)26Sortm1(EYFP)Cos/J (Abbreviated as R26R^{YFP}) with a loxP-flanked STOP sequence followed by the enhanced yellow fluorescent protein reporter gene (EYFP) inserted into the genetrap ROSA26 locus, and osxYPFCre mice expressing a fusion protein of YFP and Cre recombinase under the control of the osteoprogenitor specific osterix (Sp7) gene promoter were backcrossed at least 10 times into C57BL/6 background.

SclCreER R26R^{YFP/YFP} Hif1a^{flox/flox} mice with HSC specific tamoxifen-inducible deletion of the Hif1a gene and induction of the YPF reporter, and control SclCreER R26R^{YFP/YFP} mice with two Hif1a wild-type alleles were produced by intercrossing the three parental strains. Likewise osxCre Hif1a^{flox/flox} mice were produced by intercrossing Hif1a^{flox/flox} mice with osxCre transgenic mice and R26R^{YFP/YFP} strain. Offspring were genotyped from ear clips by PCR using allele specific primers

### Mobilization Treatments

All procedures were approved by the animal experimentation ethics committee of the University of Queensland. All experiments were performed on nine to twelve-week old C57BL/6 male mice or genetically modified mice back-crossed at least 10 times into C57BL/6. Recombinant human G-CSF (Amgen, Thousand Oaks, Calif.) was injected twice daily subcutaneously at 125 μg/kg per injection for up to 4 consecutive days. Plerixafor (AMD3100 octohydrochloride, Tocris Bioscience, Bristol, UK) was injected intraperitoneally as a single 16 mg/kg dose corresponding to 10 mg/kg of AMD3100 base. Tissues were harvested 1 h after Plerixafor administration. Compounds X and A-F were injected daily intraperitoneally at 20 mg/kg per injection with the exception of Compound D at 60 mg/kg per injection. Compounds H and J were administered at 60 mg/kg and Compound K was administered at 100 mg/kg. Control mice were injected with an equivalent volume of saline. At specified time-points, mice were anesthetized with isoflurane before cardiac puncture to harvest blood. Mice were then euthanized by cervical dislocation and BM and bones harvested.

### Induction of the SclCreER Transgene with Tamoxifen

SclCreER mice were gavaged daily with tamoxifen free base diluted in peanut oil containing 10% ethanol for three days to induce the SclCreER transgene. G-CSF was administered for three days beginning on the final day of tamoxifen gavaging. Following G-CSF treatment, mice were then euthanized by cervical dislocation and BM and bones harvested.

### Cell Counts and Colony Assays

For myeloid colony assays, 10 μL whole blood or leukocyte suspension containing 50,000 BM or spleen cells were deposited in 35 mm petri dishes and covered with 1 mL IMDM supplemented with 16% methylcellulose (high viscosity Methocell MC, Fluka Sigma-Aldrich, St Louis, Mo.) and 35% FCS. Optimal concentrations of mouse IL-3, IL-6 and soluble kit ligand were added as conditioned media from stably transfected BHK cell lines. Colonies were counted after 7 days culture at 37° C. in a humidified incubator containing 5% CO₂.

### Flow Cytometry

Following flushing with PBS plus 2% NCS, enriched central BM cells were pelleted at 370×g for 5 minutes at 4° C. and resuspended in CD16/CD32 hybridoma 2.4G2 supernatant to block IgGFc receptors. HSCs were stained with the biotinylated lineage antibody cocktail (CD3, CD5, B220, CD11b, Gr-1, Ter119) and biotinylated CD41 together with streptavidin (SAV)-Pacific Blue, anti-Sca-1-PECY7, anti-KIT-APC, CD48-FITC and CD150-PE as previously described (17). Data were acquired on a CyAn (Dako Cytomation) flow cytometer and analyzed following compensation with single color controls using FlowJo software (Tree Star, Ashland, Oreg.).

### Competitive Repopulation Assays

The content of mobilized blood samples in competitive repopulating HSC was determined in competitive repopulation assays as previously described (12,17). The day prior to blood harvest, recipient congenic B6.SJL CD45.1⁺ female mice were lethally irradiated with 11.0Gy in two split doses 4 hours apart. 50 μL blood samples from 6 mobilized CD45.2⁺ C57BL/6 donor mice were pooled within each treatment group and 20 μL blood aliquots were taken from this pool and mixed with 200,000 competitive whole BM cells from untreated B6.SJL CD45.1⁺ in a total volume of 200 μL saline, and injected retro-orbitally into each lethally irradiated recipient. Recipients were maintained with antibiotics for the first 3 weeks post-transplant and tail bled 8, 12 and 16 weeks post-transplant to determine CD45.2 (test donor blood) versus CD45.1 (competitive whole BM cells) chimerism in myeloid, B and T lineages by flow cytometry using CD45.1-PE, CD45.2-allophycocyanin (APC), CD3-FITC, B220-allophycocyanin (APC)-cyanin (CY) 7, CD11b-PECY7, Ly6G-Pacific Blue. Content in repopulating units (RU) was calculated as previously described (18-19).

### Statistical Analyses

Differences between treatment groups were analyzed using a two-tailed t-test or non-parametric Mann-Whitney depending on distribution normality. A value of p<0.05 was considered significant. Data are presented as mean±standard deviation.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. To, L. B., Levesque, J.-P. & Herbert, K. E. How I treat patients who mobilize hematopoietic stem cells poorly. Blood 118, 4530-4540 (2011).
2. Sancho, J. M., et al. Predictive factors for poor peripheral blood stem cell mobilization and peak CD34(+) cell count to guide pre-emptive or immediate rescue mobilization. Cytotherapy 14, 823-829 (2012).
3. Foudi, A., et al. Reduced retention of radioprotective hematopoietic cells within the bone marrow microenvironment in CXCR4−/− chimeric mice. Blood 107, 2243-2251 (2006).
4. Sugiyama, T., Kohara, H., Noda, M. & Nagasawa, T. Maintenance of the hematopoietic stem cell pool by CXCL12-CXCR4 chemokine signaling in bone marrow stromal cell niches. Immunity 25, 977-988 (2006).
5. Levesque, J. P., Hendy, J., Takamatsu, Y., Simmons, P. J. & Bendall, L. J. Disruption of the CXCR4/CXCL12 chemotactic interaction during hematopoietic stem cell mobilization induced by GCSF or cyclophosphamide. J Clin Invest 111, 187-196 (2003).
6. Semerad, C. L., et al. G-CSF potently inhibits osteoblast activity and CXCL12 mRNA expression in the bone marrow. Blood 106, 3020-3027 (2005).
7. Broxmeyer, H. E., et al. Rapid mobilization of murine and human hematopoietic stem and progenitor cells with AMD3100, a CXCR4 antagonist. J Exp Med 201, 1307-1318 (2005).
8. DiPersio, J. F., et al. Phase III prospective randomized double-blind placebo-controlled trial of plerixafor plus granulocyte colony-stimulating factor compared with placebo plus granulocyte colony-stimulating factor for autologous stem-cell mobilization and transplantation for patients with non-Hodgkin's lymphoma. J Clin Oncol 27, 4767-4773 (2009).
9. DiPersio, J. F., et al. Plerixafor and G-CSF versus placebo and G-CSF to mobilize hematopoietic stem cells for autologous stem cell transplantation in patients with multiple myeloma. Blood 113, 5720-5726 (2009).
10. Soriano, P. Generalized lacZ expression with the ROSA26 Cre reporter strain. Nat Genet 21, 70-71 (1999).
11. Gothert, J. R., et al. In vivo fate-tracing studies using the Scl stem cell enhancer: embryonic hematopoietic stem cells significantly contribute to adult hematopoiesis. Blood 105, 2724-2732 (2005).
12. Winkler, L. G., et al. Bone marrow macrophages maintain hematopoietic stem cell (HSC) niches and their depletion mobilizes HSCs. Blood 116, 4815-4828 (2010).
13. Christopher, M. J. & Link, D. C. Granulocyte colony-stimulating factor induces osteoblast apoptosis and inhibits osteoblast differentiation. Journal Bone Miner Res 23, 1765-1774 (2008).
14. Katayama, Y., et al. Signals from the sympathetic nervous system regulate hematopoietic stem cell egress from bone marrow. Cell 124, 407-421 (2006).
15. Levesque, J. P., Helwani, F. M. & Winkler, I. G. The endosteal ‘osteoblastic’ niche and its role in hematopoietic stem cell homing and mobilization. Leukemia 24, 1979-1992 (2010).
16. Raaijmakers, M. H. G. P., et al. Bone progenitor dysfunction induces myelodysplasia and secondary leukaemia. Nature 464, 852-857 (2010).
17. Barbier, V., Winkler, I. G. & Levesque, J. P. Mobilization of hematopoietic stem cells by depleting bone marrow macrophages. Methods Mol Biol 904, 117-138 (2012).
18. Purton, L. E. & Scadden, D. T. Limiting factors in murine hematopoietic stem cell assays Cell Stem Cell 1, 262-270 (2007).
19. Winkler, L. G., et al Hematopoietic stem cell mobilizing agents G-CSF, cyclophosphamide or AMD3100 have distinct mechanisms of action on bone marrow HSC niches and bone formation. Leukemia 26, 1594-1601 (2012).

1-13. (canceled)

14. A method for mobilizing hematopoietic stem cells and/or progenitor cells from bone marrow into peripheral blood of a donor subject, the method comprising, consisting or consisting essentially of: administering concurrently to the subject a HIF-α potentiating agent and at least one mobilizer of hematopoietic stem cells and/or progenitor cells in effective amounts to mobilize hematopoietic stem cells and/or progenitor cells from the bone marrow into the peripheral blood of the subject.

15. The method of claim 14, wherein the subject has an immunocompromised condition or is at risk of acquiring an immunocompromised condition.

16. The method of claim 14, wherein the subject has a hyperproliferative cell disorder, and has been, is or will be subjected to a medical treatment that gives rise or is likely to give rise to an immunocompromised condition.

17. The method of claim 16, wherein the hyperproliferative cell disorder is a cancer or an autoimmune disease.

18. The method of claim 16, wherein the hyperproliferative cell disorder is a cancer selected from leukemia, multiple myeloma, or lymphoma.

19. The method of claim 14, further comprising collecting or harvesting mobilized hematopoietic stem cells and/or progenitor cells from the subject.

20. The method of claim 19, further comprising culturing and/or storing the collected or harvested mobilized hematopoietic stem cells and/or progenitor cells.

21. The method of claim 19, further comprising transplanting the collected or harvested mobilized hematopoietic stem cells and/or progenitor cells into a recipient subject.

22. The method of claim 21, wherein the recipient subject is the donor of the collected or harvested mobilized hematopoietic stem cells and/or progenitor cells and the transplantation is an autologous transplantation.

23. The method of claim 21, wherein the recipient subject is not the donor of the collected or harvested mobilized hematopoietic stem cells and/or progenitor cells and the transplantation is a syngeneic, allogeneic or xenogeneic transplantation.

24. The method of claim 21, wherein the recipient subject has an immunocompromised condition or has been exposed to a medical treatment that results in an immunocompromised condition.

25. The method of claim 21, further comprising administering to the recipient subject prior to, simultaneously with, or after the stem cell transplantation a HIF-α potentiating agent and a mobilizer of hematopoietic stem cells and/or progenitor cells in effective amounts to mobilize hematopoietic stem cells and/or progenitor cells from the bone marrow into the peripheral blood of the subject.

26. The method of claim 14, wherein the HIF-α potentiating agent is an inhibitor of a HIF prolyl hydroxylase (PHD).

27. The method of claim 26, wherein the inhibitor of HIF prolyl hydroxylase is a heterocyclic carboxamide compound.

28. The method of claim 27, wherein the heterocyclic carboxamide compound is a heterocyclic carbonyl glycine.

29. The method of claim 26, wherein the inhibitor of HIF prolyl hydroxylase is selected from the group consisting of [(4-Hydroxy-7-phenylsulfanyl-isoquinoline-3-carbonyl)-amino]-acetic acid, {[5-(4-Chloro-phenoxy)-1-cyano-4-hydroxy-isoquinoline-3-carbonyl]-amino}-acetic acid, [(1-Cyano-4-hydroxy-5-phenoxy-isoquinoline-3-carbonyl)-amino]-acetic acid, {[7-Cyano-1-(2-fluoro-benzyl)-4-hydroxy-1H-pyrrolo[2,3-c]pyridine-5-carbonyl]-amino}-acetic acid, [(1,3-Dicyclohexyl-6-hydroxy-2,4-dioxo-1,2,3,4-tetrahydro-pyrimidine-5-carbonyl)-amino]-acetic acid, {[2-(3,4′-Difluoro-biphenyl-4-ylmethyl)-5-hydroxy-6-isopropyl-3-oxo-2,3-dihydro-pyridazine-4-carbonyl]-amino}-acetic acid, 2-(6-Morpholin-4-yl-pyrimidin-4-yl)-4-[1,2,3]triazol-1-yl-1,2-dihydro-pyrazol-3-one, [(4-Hydroxy-7-phenoxy-isoquinoline-3-carbonyl)-amino]-acetic acid, {[4-Hydroxy-7-(4-methoxy-phenoxy)-isoquinoline-3-carbonyl]-amino}-acetic acid, and {[5-(3-Fluoro-phenyl)-3-hydroxy-pyridine-2-carbonyl]-amino}-acetic acid.

30. The method of claim 26, wherein the inhibitor of HIF prolyl hydroxylase is a compound of Formula (I), a compound of Formula (II), a compound of Formula (III), or a compound of Formula (IV).

31. The method of claim 14, wherein the at least one mobilizer is selected from a colony stimulating factor, a CXCR4 antagonist, or a combination thereof.

32. The method of claim 14, wherein the at least one mobilizer is G-CSF.

33. The method of claim 14, wherein the at least one mobilizer is Plerixafor.

34. The method of claim 14, wherein the HIF-α potentiating agent is an inhibitor of a HIF prolyl hydroxylase, and the at least one mobilizer comprises a colony-stimulating factor.

35. The method of claim 34, wherein the colony-stimulating factor is G-CSF.

36. The method of claim 35, wherein the at least one mobilizer further comprises Plerixafor.

37. The method of claim 14, wherein the mobilizer(s) and the HIF-α potentiating agent are administered simultaneously to the subject.

38. The method of claim 14, wherein the HIF-α potentiating agent is administered to the subject prior to administration of the mobilizer.

39. The method of claim 14, wherein the HIF-α potentiating agent is administered after administration of the mobilizer to the subject.

40. A method for increasing the dose of a medicament in a subject, wherein the medicament results or increases the risk of developing an immunocompromised condition, the method comprising, consisting or consisting essentially of: administering concurrently to the subject the medicament in a dose that ordinarily induces side effects, together with at least one mobilizer of hematopoietic stem cells and/or progenitor cells and a HIF-α potentiating agent in amounts effective for inhibiting or preventing the induction of those side effects.

41-50. (canceled)

51. A method for mobilizing hematopoietic stem cells and/or progenitor cells from bone marrow into peripheral blood of a donor subject, the method comprising, administering to the subject a HIF-α potentiating agent in an effective amount to mobilize hematopoietic stem cells and/or progenitor cells from the bone marrow into the peripheral blood of the subject.

52-53. (canceled)
